SEQUENCE LISTING (52 sequences)

SEQ ID NO: 1 - DNA, 58 bp, Artificial Sequence
  Description of Artificial Sequence Synthetic oligonucleotide
ccggcctgacaacctttacacctttctcgagaaaggtgtaaaggttgtcaggtttttg

SEQ ID NO: 2 - DNA, 58 bp, Artificial Sequence
  Description of Artificial Sequence Synthetic oligonucleotide
ccgggcctgacaacctttacaccttctcgagaaggtgtaaaggttgtcaggctttttg

SEQ ID NO: 3 - DNA, 23 bp, Artificial Sequence
  Description of Artificial Sequence Synthetic oligonucleotide
gtgtgcaccaccggacctcctgg

SEQ ID NO: 4 - DNA, 23 bp, Artificial Sequence
  Description of Artificial Sequence Synthetic oligonucleotide
gctccagcatccaggaggtccgg

SEQ ID NO: 5 - DNA, 60 bp, Artificial Sequence
  Description of Artificial Sequence Synthetic oligonucleotide
  CDS (1)..(60)
tgctccagcatccaggaggtccggcggtgcacacggcttgagatgcctgacaacctttac

SEQ ID NO: 6 - protein, 20 aa, Artificial Sequence
  Description of Artificial Sequence Synthetic peptide
CSSIQEVRRCTRLEMPDNLY

SEQ ID NO: 7 - DNA, 59 bp, Artificial Sequence
  Description of Artificial Sequence Synthetic oligonucleotide
tgctccagcatccaggagtccggcggtgcacacggcttgagatgcctgacaacctttac

SEQ ID NO: 8 - DNA, 59 bp, Artificial Sequence; identical to SEQ ID NO: 7
  Description of Artificial Sequence Synthetic oligonucleotide

SEQ ID NO: 9 - DNA, 49 bp, Artificial Sequence
  Description of Artificial Sequence Synthetic oligonucleotide
tgctccagcatccaggagcacacggcttgagatgcctgacaacctttac

SEQ ID NO: 10 - DNA, 56 bp, Artificial Sequence
  Description of Artificial Sequence Synthetic oligonucleotide
tgctccagcatccagtccggcggtgcacacggcttgagatgcctgacaacctttac

SEQ ID NO: 11 - DNA, 52 bp, Artificial Sequence
  Description of Artificial Sequence Synthetic oligonucleotide
tgctccagcatccaggaggtgcacacggcttgagatgcctgacaacctttac

SEQ ID NO: 12 - DNA, 59 bp, Artificial Sequence; identical to SEQ ID NO: 7
  Description of Artificial Sequence Synthetic oligonucleotide

SEQ ID NO: 13 - DNA, 20 bp, Artificial Sequence
  Description of Artificial Sequence Synthetic oligonucleotide
gctccagcatccaggaggtc

SEQ ID NO: 14 - DNA, 28 bp, Artificial Sequence
  Description of Artificial Sequence Synthetic oligonucleotide
ccaccggacctcctggatgctggagctg

SEQ ID NO: 15 - DNA, 28 bp, Artificial Sequence
  Description of Artificial Sequence Synthetic oligonucleotide
cagctccagcatccaggaggtccggtgg

SEQ ID NO: 16 - protein, 29 aa, Artificial Sequence
  Description of Artificial Sequence Synthetic endosomolytic/fusogenic peptide
AALEALAEALEALAEALEALAEAAAAGGC

SEQ ID NO: 17 - protein, 30 aa, Artificial Sequence
  Description of Artificial Sequence Synthetic endosomolytic/fusogenic peptide
AALAEALAEALAEALAEALAEALAAAAGGC

SEQ ID NO: 18 - protein, 15 aa, Artificial Sequence
  Description of Artificial Sequence Synthetic endosomolytic/fusogenic peptide
ALEALAEALEALAEA

SEQ ID NO: 19 - protein, 22 aa, Artificial Sequence
  Description of Artificial Sequence Synthetic endosomolytic/fusogenic peptide
GLFEAIEGFIENGWEGMIWDYG

SEQ ID NO: 20 - protein, 23 aa, Artificial Sequence
  Description of Artificial Sequence Synthetic endosomolytic/fusogenic peptide
GLFGAIAGFIENGWEGMIDGWYG

SEQ ID NO: 21 - protein, 48 aa, Artificial Sequence
  Description of Artificial Sequence Synthetic endosomolytic/fusogenic peptide
GLFEAIEGFIENGWEGMIDGWYGCGLFEAIEGFIENGWEGMIDGWYGC

SEQ ID NO: 22 - protein, 44 aa, Artificial Sequence
  Description of Artificial Sequence Synthetic endosomolytic/fusogenic peptide
GLFEAIEGFIENGWEGMIDGGCGLFEAIEGFIENGWEGMIDGGC

SEQ ID NO: 23 - protein, 35 aa, Artificial Sequence
  Description of Artificial Sequence Synthetic endosomolytic/fusogenic peptide
GLFGALAEALAEALAEHLAEALAEALEALAAGGSC

SEQ ID NO: 24 - protein, 34 aa, Artificial Sequence
  Description of Artificial Sequence Synthetic endosomolytic/fusogenic peptide
GLFEAIEGFIENGWEGLAEALAEALEALAAGGSC

SEQ ID NO: 25 - protein, 41 aa, Artificial Sequence
  Description of Artificial Sequence Synthetic endosomolytic/fusogenic peptide
  MOD_RES (17)..(17): Norleucine
  MOD_RES (38)..(38): Norleucine
GLFEAIEGFIENGWEGXIDGKGLFEAIEGFIENGWEGXIDG

SEQ ID NO: 26 - protein, 19 aa, Artificial Sequence
  Description of Artificial Sequence Synthetic endosomolytic/fusogenic peptide
LFEALLELLESLWELLLEA

SEQ ID NO: 27 - protein, 20 aa, Artificial Sequence
  Description of Artificial Sequence Synthetic endosomolytic/fusogenic peptide
GLFKALLKLLKSLWKLLLKA

SEQ ID NO: 28 - protein, 20 aa, Artificial Sequence
  Description of Artificial Sequence Synthetic endosomolytic/fusogenic peptide
GLFRALLRLLRSLWRLLLRA

SEQ ID NO: 29 - protein, 30 aa, Artificial Sequence
  Description of Artificial Sequence Synthetic endosomolytic/fusogenic peptide
WEAKLAKALAKALAKHLAKALAKALKACEA

SEQ ID NO: 30 - protein, 22 aa, Artificial Sequence
  Description of Artificial Sequence Synthetic endosomolytic/fusogenic peptide
GLFFEAIAEFIEGGWEGLIEGC

SEQ ID NO: 31 - protein, 26 aa, Artificial Sequence
  Description of Artificial Sequence Synthetic endosomolytic/fusogenic peptide
GIGAVLKVLTTGLPALISWIKRKRQQ

SEQ ID NO: 32 - protein, 8 aa, Artificial Sequence
  Description of Artificial Sequence Synthetic endosomolytic/fusogenic peptide
HHHHHWYG

SEQ ID NO: 33 - protein, 10 aa, Artificial Sequence
  Description of Artificial Sequence Synthetic endosomolytic/fusogenic peptide
CHKKKKKKHC

SEQ ID NO: 34 - protein, 16 aa, Unknown
  Description of Unknown Cell permeation peptide
RQIKIWFQNRRMKWKK

SEQ ID NO: 35 - protein, 14 aa, Unknown
  Description of Unknown Cell permeation peptide
GRKKRRQRRRPPQC

SEQ ID NO: 36 - protein, 27 aa, Unknown
  Description of Unknown Cell permeation peptide
GALFLGWLGAAGSTMGAWSQPKKKRKV

SEQ ID NO: 37 - protein, 18 aa, Unknown
  Description of Unknown Cell permeation peptide
LLIILRRRIRKQAHAHSK

SEQ ID NO: 38 - protein, 26 aa, Unknown
  Description of Unknown Cell permeation peptide
GWTLNSAGYLLKINLKALAALAKKIL

SEQ ID NO: 39 - protein, 18 aa, Unknown
  Description of Unknown Cell permeation peptide
KLALKLALKALKAALKLA

SEQ ID NO: 40 - protein, 9 aa, Unknown
  Description of Unknown Cell permeation peptide
RRRRRRRRR

SEQ ID NO: 41 - protein, 10 aa, Unknown
  Description of Unknown Cell permeation peptide
KFFKFFKFFK

SEQ ID NO: 42 - protein, 37 aa, Unknown
  Description of Unknown Cell permeation peptide
LLGDFFRKSKEKIGKEFKRIVQRIKDFLRNLVPRTES

SEQ ID NO: 43 - protein, 31 aa, Unknown
  Description of Unknown Cell permeation peptide
SWLSKTAKKLENSAKKRISEGIAIAIQGGPR

SEQ ID NO: 44 - protein, 30 aa, Unknown
  Description of Unknown Cell permeation peptide
ACYCRIPACIAGERRYGTCIYQGRLWAFCC

SEQ ID NO: 45 - protein, 36 aa, Unknown
  Description of Unknown Cell permeation peptide
DHYNCVSSGGQCLYSACPIFTKIQGTCYRGKAKCCK

SEQ ID NO: 46 - protein, 42 aa, Unknown
  Description of Unknown Cell permeation peptide
RRRPRPPYLPRPRPPPFFPPRLPPRIPPGFPPRFPPRFPGKR

SEQ ID NO: 47 - protein, 13 aa, Unknown
  Description of Unknown Cell permeation peptide
ILPWKWPWWPWRR

SEQ ID NO: 48 - protein, 16 aa, Unknown
  Description of Unknown Cell permeation peptide
AAVALLPAVLLALLAP

SEQ ID NO: 49 - protein, 11 aa, Unknown
  Description of Unknown Cell permeation peptide
AALLPVLLAAP

SEQ ID NO: 50 - protein, 12 aa, Unknown
  Description of Unknown Cell permeation peptide
RKCRIVVIRVCR

SEQ ID NO: 51 - protein, 575 aa, Homo sapiens
MNGPALQPSSPSSAPSASPAAAPRGWSEFCELHAVAAARELARQYWLFAREHPQHAPLRAELVSLQFTDLFQRYFCREVRDGRAPGRDYRDTGRGPPAKAEASPEPGPGPAAPGLPKARSSEELAPPRPPGPCSFQHFRRSLRHIFRRRSAGELPAAHTAAAPGTPGEAAETPARPGLAKKFLPWSLAREPPPEALKEAVLRYSLADEASMDSGARWQRGRLALRRAPGPDGPDRVLELFDPPKSSRPKLQAACSSIQEVRWCTRLEMPDNLYTFVLKVKDRTDIIFEVGDEQQLNSWMAELSECTGRGLESTEAEMHIPSALEPSTSSSPRGSTDSLNQGASPGGLLDPACQKTDHFLSCYPWFHGPISRVKAAQLVQLQGPDAHGVFLVRQSETRRGEYVLTFNFQGIAKHLRLSLTERGQCRVQHLHFPSVVDMLHHFQRSPIPLECGAACDVRLSSYVVVVSQPPGSCNTVLFPFSLPHWDSESLPHWGSELGLPHLSSSGCPRGLSPEGLPGRSSPPEQIFHLVPSPEELANSLQHLEHEPVNRARDSDYEMDSSSRSHLRAIDNQYTPL

SEQ ID NO: 52 - DNA, 5425 bp, Homo sapiens
cccgggccaccgcctccgcccggctgcccgcccggactgtcgcggcccgcggtggcgacggcggccgctgcaaagtttccccggcggcggcggcccgggggcgcatcctcccgcaactgtcaagcgctggcggcggaaatgatgaggcgctggccattttccgagcccgggtttcctgcctgagccccgctcgagcgagccgcgagcgaggagccggcgggcgggagaggacgcgcccagggcgggggcccgcccgccccctcgggatttcgagggcccgggggcgcgcgacgccatgggccggccgggcccagagctcctgtctctcagcccggccgcaccacctgggtctccgccatgaacgggcctgccctgcagccctcctcgccctcttccgcgccctcagcctccccggcggcggccccgcggggctggagcgagttctgtgagttgcacgccgtagcggcggcccgggagctggcccgccagtactggctgttcgcccgggagcatccgcagcacgcgccgctgcgcgccgagctggtgtcgctgcagttcaccgacctcttccagcgctacttctgccgcgaggtgcgcgacggacgggcgccgggccgcgactaccgggacacaggccgtgggcccccagccaaggccgaggcgtccccggagccaggccccggccccgccgcccctggcctgcccaaggcccgcagctctgaggagctggccccgccgcggccgcccgggccctgctccttccagcactttcgccgcagcctccgccacatcttccgccgccgctcggccggggagctgccagcggcccacaccgctgccgcccccgggacccccggagaggctgctgagacccccgcccggcctggcctggccaagaagttcctgccctggagcctggcccgggagccgccacccgaggcgctgaaggaggcggtgctgcgctacagcctggccgacgaggcctccatggacagcggggcacgctggcagcgcgggaggctggcgctgcgccgggccccgggccccgatggccccgaccgcgtgctggagctcttcgacccacccaagagttcaaggcccaagctacaagcagcttgctccagcatccaggaggtccggtggtgcacacggcttgagatgcctgacaacctttacacctttgtgctgaaggtgaaggaccggacagacatcatctttgaggtgggagacgagcagcagctgaattcatggatggctgagctctcggagtgcacaggccgagggctggagagcacagaagcagagatgcatattccctcagccctagagcctagcacgtccagctccccaaggggcagcacagattcccttaaccaaggtgcttctcctggggggctgctggacccggcctgccagaagacggaccatttcctgtcctgctacccctggttccacggccccatctccagagtgaaagcagctcagctggttcagctgcagggccctgatgctcatggagtgttcctggtgcggcagagcgagacgcggcgtggggaatacgtgctcactttcaactttcaggggatagccaagcacctgcgcctgtcgctgacagagcggggccagtgccgtgtgcagcacctccactttccctcggtcgtggacatgctccaccacttccagcgctcgcccatcccactcgagtgcggcgccgcctgtgatgtccggctctccagctacgtggtagtcgtctcccaaccaccaggttcctgcaacacggtcctcttccctttctcccttcctcactgggattcagagtcccttcctcactggggttcagagttgggccttccccaccttagttcttctggctgtccccgggggctcagcccagagggtctcccagggcgatcctcaccccccgagcagatcttccacctggtgccttcgcccgaagaactggccaacagcctgcagcacctggagcatgagcctgtgaatcgagcccgggactcggactacgaaatggactcatcctcccggagccacctgcgggccatagacaatcagtacacacctctctgaccagtgaggaattccaggcctcaacagctgcccttgaggagcacaggcagaagtgtgaacttgtgaatgtaattgatctttccttccttccagagaaagatttaagggacactgttaactgctcgtgccagtttggaagtgacccttctattaggcctgttgaagggccctcctgtaggtttcatctatccacctggctttctccttattgtttacagatgtagttcttgttagaggatgccgctagctcctgcccggggtccctatgcccagtccccgttactcttagagaaaggagttggggtgagggccagagctggcagtggaaacttgttctctttttcactgacactgtcacagcggatgacagactttctacggggaggagggggggatcatcaggaagcccagaacactaacaagcggttctcccatctaccgtcagtccacatggcaggtctgctgtgtccacaccacagatgaccacatctaatcctgcttctactctcagctttaggacaaaagctctgtcagaggcacaagctgaaggtcaaaaatgatttaaaacattttacctcagactaatttctttaaaggattcaggttcaaaacttaaccactgcttatttcagtgcactgtttcaactaacacccatgctatttttgtagtcagaaacagctatgcaaaccctacctaatttacagtctgagccagcatgctggcttgtctactgcatcctcgggacagtcacctgccactgagtggccactgtccttcctaaatgtcaagaagtgaagtatgtcaccctttcagggaaattcaggcaattactgaaataggagggtggcaagaacagttctatcctggtgccttacgaataaaaaactggattctggtttacagcagctttacagtgatagttaaattaactggggctaggggaagagcaagcaaaaagggaagaaggactcctaggccctttctagtaaatccttcagcaacaaggctggcttggtgccctccaagcatctaatggcttattaaattatcccacaagtgggttttaggctccttttttgagccaaaatggaagctgggaatctggtgccataactaatgagaaactcctttaatagcccacaatcagtgttctgttctagctggctactgcttcactggattgagaatctatctatctccttgcacacatgggcacacacaatctccaccatccagggaggtcctgaagtcaaatctctatctatacaagtgatacaattcatagggggctggctcctcccagaacctgtctggaggctcagaaacgggggcagtgacagtggagtcagctgctcttgggtgccagcagagccattcagtacaacccccaggctcacagcagtggcttctaggaaactgggagtttagatcagctttacagatacatcgatcagaggctaaaatgaaacctcagcctaaaactcataggactgactgcctgggaggagggttaggtctgcttcttccacttatacttagtctctgtgctccaagaggtcaaatttttgcttctagaatttccttggggtctttcagagggtgggggaacaaacccctatgcacttttcttttttttttttttgagatggagtttctcttgtcaaccgggctggagtgcagtggtgcaatcttggctcactgcaacctccaccttcctggttcaagcgattctgcctcgacctctcaagtagctgggattacaagcaccagccaccatgcctggctaattttgtatttttagtagagacagggtttcaccatgttggccaggctggtctcgaatgtctgacctcaggtgatccacccgccttggcctcccaaagtgctgggattacaggcgcgagccaccgcgcccagcctacaccacttttagtaccaacactcttgggtgatttcatggaccctaaagcagacctgacactgatccagatttgcagtccatttttaaggacacctgtctttatttcctcaaagtcaagcagctttctctggaaaatgaatgctaattagtgtgaaccaaaagagtaagtaagagtctgaagtttttttaaaggagaaagcttattatggaaagtcactggtcctcccctccgcacaggaaaggtacccagtagataatgaaccaaattaagttccctccctccagccagaagttaaacatctgggatatgacgtcttcatgccaggggcactcatttcttagcagcctctctacatacatctctcaggtggtgccaagaggcacaccaggtagagcaaacttagcagctctgactaacaggctgcaaagtgcaagttcagattctgtggcagagatttggaaggcacccacctccagactgcttcccgtccaagttaccaggacagctcaaaaacatgctgacagaaaactcccatggctctaggaagaagtgacactaagccaacacctttctttatgtgggagcagaatcagctgatgaaggggtgggcagcagtgtggggcaggcaccccactggctgcagctagcccaccataggcacagcacatcccaccactctccttccagtcctgaccaggccccagccggcaacttctaccgagagccatggctcaacaccaaactggacagtagacatcatgatccctccagttagctctaattacagaccccaccagtacagcttgacagctcccggcaccatcccttccttcatctgacttattgaacttttacaaactaacagtcaccagcaccaaagaattaagtcaactaacctgccttgaattttagaccagcaatccatatggctttatctggtataaatcttctgcctttgatcatttctggaccgtaggaaaaaggaatagcaatcattaaaatcttgggccagagaacactatttttacataacagtttcttaacctaaagtcaaggccttggactcttccctgagggttgcctgagattccttcatgctttctattcaggactaagtcccttactgcaaatgtgttagctctaacatctcccacaagctagaggaacttgcgagtatattaacaaggacacatctgacatcctgtgtttggttagaatatacagcacattgtgataacataaagtggattcatcttgtatcattataggcagaaggtatttggcaaatttttatgtattgttttatgtactgtacaagtaacttattcttgaataatgcaaattttgctataatgtacaaattgctatatgtgaattaaaaagttttcagaatcttgaaaaaaaaaaaaaaaaaaaa

(19) United States
(12) Patent Application Publication
(10) Pub. No.: US 2017/0355958 A1
(43) Pub. Date: Dec. 14, 2017

(54) MODULATION OF SH2B3 TO IMPROVE RED BLOOD CELL PRODUCTION FROM STEM CELLS AND/OR PROGENITOR CELLS

(71) Applicant: CHILDREN'S MEDICAL CENTER CORPORATION, Boston, MA (US)

(72) Inventor: Vijay G. SANKARAN, Jamaica Plain, MA (US)

(73) Assignee: CHILDREN'S MEDICAL CENTER CORPORATION, Boston, MA (US)

(21) Appl. No.: 15/529,220

(22) PCT Filed: Nov. 24, 2015
(86) PCT No.: PCT/US15/62333

§ 371 Date: May 24, 2017

Related U.S. Application Data
(60) Provisional application No. 62/083,439, filed on Nov. 24, 2014.

(51) Int. Cl. C12N 5/078; A61K 35/18

(52) U.S. Cl. CPC C12N 5/0641; A61K 35/18; C12N 2501/415; C12N 2501/165; C12N 2506/03; C12N 2501/155; C12N 2501/125; C12N 2501/2306; C12N 2501/115; C12N 2501/26

(57) ABSTRACT

Disclosed herein are methods for producing red blood cells (RBCs) from a population of stem cells and/or progenitor cells. In at least one of the stem cells or progenitor cells, SH2B3 protein activity is decreased, SH2B3 mRNA level is decreased, and/or SH2B3 protein level is decreased. The methods provided herein permit the production of RBCs with increased quantity and/or quality as compared to a method using the same population of stem cells and/or progenitor cells without SH2B3 inhibition or disruption. Also provided herein are methods of use of the RBCs produced using the methods described herein.

### CROSS REFERENCE TO RELATED APPLICATIONS

This application claims priority under 35 U.S.C. 119(e) of U.S. Provisional Patent Application Ser. No: 62/083,439 filed on Nov. 24, 2014, the contents of which is incorporated herein by reference in its entirety.

### GOVERNMENT SUPPORT

This invention was made with government support under grant No. R01DK103794 and R21HL120791 awarded by the National Institutes of Health. The government has certain rights in the invention.

### TECHNICAL FIELD

The present invention relates to red blood cell production from stem cells and/or progenitor cells.

### BACKGROUND

Transfusion of red blood cells (RBCs) is routinely used for many clinical and surgical applications. According to the American Association of Blood Banks, 29 million units of blood components are transfused annually in the United States. This procedure has saved many lives. The demand for such transfusions continues to increase with advances in medical treatments and an aging population.

Donated blood is a major source of RBCs. However, the amount and/or quality of donated blood are not guaranteed. Production of red blood cells (RBCs) ex vivo using hematopoietic and pluripotent stem cell sources is an area of major interest as possible transfusion replacements. RBCs produced ex vivo have been successfully transfused into recipients in clinical trials demonstrating the effectiveness of this approach (Giarratana et al., Blood, 2011).

There is significant excitement regarding the use of stem cells as a source for cell replacement therapies (Fox et al., 2014). In vitro differentiated red blood cells (RBCs) from hematopoietic stem and progenitor cells (HSPCs) have been successfully transfused into recipients in clinical trials (Giarratana et al., 2011; Migliaccio et al., 2012), and similar approaches using pluripotent stem cells (PSCs), including human embryonic stem cells (hESCs) and induced PSCs (iPSCs), are under investigation (Kobari et al., 2012; Slukvin, 2013; Sturgeon et al., 2013). However, utilizing these stem cell sources for RBC production is limited by low yields of fully mature cells, making the process costly and inefficient (Migliaccio et al., 2012; Rousseau et al., 2014). Overcoming these major limitations could enhance the ability to manage the blood supply (Williamson and Devine, 2013). Accordingly, a major limitation that exists in RBC ex vivo production is the ability to produce a sufficient amount of RBCs for therapeutic purposes. Typically only a fraction of a unit can be produced in a cost effective manner.

Accordingly, there is an unmet need in the art for improved methods to produce RBCs ex vivo from stem cells and/or progenitor cells.

### SUMMARY

The invention is based, in part, on the discovery that inhibition of the SH2B3 gene in a population of stem cells and/or progenitor cells can increase the amount and quality of red blood cells (RBCs) differentiated from the population of stem cells and/or progenitor cells. In particular, the inventor has demonstrated using shRNA or CRISPR/Cas9 to knock down SH2B3 expression, that hematopoietic stem and progenitor cells (HSPCs) can be differentiated into mature RBCs, as shown by an increase in CD235a marker and a decrease in CD71 surface marker. And thus the inventor has discovered that inhibition of SH2B3 can be used to produce RBCs ex vivo from a population of stem cells and/or progenitor cells.

Accordingly, in one aspect, the invention provides a method of producing red blood cells (RBCs) ex vivo from a population of stem cells and/or progenitor cells, the method comprising: (i) inhibiting SH2B3 in the population of stem cells and/or progenitor cells; (ii) culturing the population of stem cells and/or progenitor cells for a time sufficient to induce differentiation of at least one stem cell or progenitor cell to a RBC; and (iii) collecting a population of RBCs.

In some embodiments, the inhibiting SH2B3 decreases SH2B3 protein level, SH2B3 mRNA level, SH2B3 protein activity, or combinations thereof.

In some embodiments, the inhibiting SH2B3 comprises contacting the population of stem cells and/or progenitor cells with a genome-editing agent for targeted excision of the SH2B3 gene from at least one stem cell or progenitor cell.

In some embodiments, the inhibiting SH2B3 comprises contacting the population of stem cells and/or progenitor cells with an antagonist of SH2B3.

In one aspect, the invention provides a method of inducing a population of stem cells and/or progenitor cells to differentiate into red blood cells (RBCs), the method comprising: (i) contacting the population of stem cells and/or progenitor cells with an antagonist of SH2B3 and culturing the population of stem cells and/or progenitor cells for a time sufficient to induce the differentiation of at least one stem cell or progenitor cell into a RBC, wherein the antagonist of SH2B3 decreases the activity of the SH2B3 protein or decreases SH2B3 mRNA or protein levels; and (ii) collecting a population of RBCs.

In one aspect, the invention provides a method of inducing a population of stem cells and/or progenitor cells to differentiate into red blood cells (RBCs), the method comprising: (i) contacting the population of stem cells and/or progenitor cells with a genome-editing agent and culturing the population of stem cells and/or progenitor cells for a time sufficient to induce the differentiation of at least one stem cell or progenitor cell into a RBC, wherein the genome-editing agent excises the SH2B3 gene from at least one stem cell or progenitor cell; and (ii) collecting a population of RBCs.

In some embodiments of any of the foregoing aspects, the genome-editing agent is selected from the group consisting of a Zinc-Finger Nuclease (ZFN), a Clustered Regularly Interspaced Short Palindromic Repeats (CRISPR)/CRISPR associated (Cas) system, and a Transcription Activator-Like Effector Nuclease (TALEN).

In some embodiments of any of the foregoing aspects, the genome-editing agent is present in a vector.

In some embodiments of any of the foregoing aspects, the antagonist of SH2B3 is selected from the group consisting of an inorganic molecule, an organic molecule, a nucleic acid, a nucleic acid analog or derivative, a peptide, a peptidomimetic, a protein, an antibody or an antigen-binding fragment thereof, and combinations thereof.

In some embodiments of any of the foregoing aspects, the antagonist of SH2B3 specifically binds to SH2 domain, PH domain, or both the SH2 and PH domains of the SH2B3 protein.

In some embodiments of any of the foregoing aspects, the antagonist of SH2B3 is a RNAi agent that inhibits the expression of SH2B3.

In some embodiments of any of the foregoing aspects, the RNAi agent is a siRNA, shRNA, dsRNA that hybridizes to SH2B3 mRNA.

In some embodiments of any of the foregoing aspects, the population of stem cells and/or progenitor cells is selected from the group consisting of hematopoietic stem cells, hematopoietic progenitor cells, pluripotent stem cells, induced pluripotent stem cells (iPSCs), embryonic stem cells, and combinations thereof.

In some embodiments of any of the foregoing aspects, the method increases the expansion of RBCs from the population of stem cells and/or progenitor cells.

In some embodiments of any of the foregoing aspects, the method increases the quality of RBCs.

In some embodiments of any of the foregoing aspects, the population of stem cells and/or progenitor cells is of mammalian origin.

In some embodiments of any of the foregoing aspects, the population of stem cells and/or progenitor cells is of human origin.

In some embodiments of any of the foregoing aspects, the RBCs are isolated by leukocyte filtration or flow cytometric sorting.

In some embodiments of any of the foregoing aspects, the population of stem cells and/or progenitor cells is obtained from a donor subject.

In some embodiments of any of the foregoing aspects, the population of stem cells and/or progenitor cells is derived from peripheral blood mononuclear cells, cord blood, bone marrow, cord tissue, or G-CSF mobilize peripheral blood of the donor subject.

In some embodiments of any of the foregoing aspects, the method further comprises administering a population of RBCs to a subject in need thereof, wherein the RBCs are produced from the population of stem cells and/or progenitor cells obtained from the donor subject.

In some embodiments of any of the foregoing aspects, the population of stem cells is iPSCs.

Another aspect of the invention relates to a population of RBCs produced according to the methods described herein.

Another aspect of the invention relates to an admixture comprising a population of RBCs and a population of stem cells and/or progenitor cells, wherein the RBCs are produced according to the methods described herein.

Another aspect of the invention relates to a blood bank comprising a population of RBCs produced according to the methods described herein.

Another aspect of the invention relates to a cell culture media comprising a population of stem cells and/or progenitor cells, at least one RBC differentiated from at least one stem cell or progenitor cell, and an antagonist of SH2B3.

Another aspect of the invention relates to a method of administering a population of RBCs to a subject, comprising administering an effective amount of RBCs to the subject, wherein the RBCs have been contacted ex vivo or in vitro with an effective amount of an antagonist of SH2B3, wherein the antagonist of SH2B3 decreases the activity of the SH2B3 protein or decreases SH2B3 mRNA or protein levels.

Another aspect of the invention relates to a method of administering a population of RBCs to a subject, comprising administering an effective amount of RBCs to the subject, wherein the RBCs are produced from a population of stem cells and/or progenitor cells having been contacted ex vivo or in vitro with an effective amount of a genome-editing agent, wherein the genome-editing agent excises the SH2B3 gene from at least one stem cell or progenitor cell.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1C shows a three-phase culture system to allow synchronous differentiation of CD34+ hematopoietic stem and progenitor cells (HSPCs) from cord blood and adult (bone marrow and mobilized peripheral blood) sources. FIG. 1A shows a simplified scheme illustrating the method of differentiation of human CD34+ HSPCs into mature RBCs using inhibition of SH2B3. FIG. 1B shows a Western blot showing SH2B3 protein levels 8 days following infection with sh83 (SEQ ID NO: 1) and sh84 (SEQ ID NO: 2) SH2B3 shRNAs, showing sh83 and sh84 shRNAs effectively reduce the level of SH2B3s protein in the cultures. FIG. 1C shows representative cytocentrifuge images of May-Grünwald-Giemsa stained SH2B3-KD (sh83 and/or sh84 SH2B3 shRNA treated) cells at the indicated days of differentiation.

FIG. 2 shows that the cells undergoing differentiation were assessed using phenotypic surface markers CD71 and CD235a, in a fairly normal manner when compared to controls.

FIGS. 3A-3B shows that the cells differentiate more effectively toward becoming mature RBCs with reduced levels of SH2B3. FIG. 3A shows a representative histogram plots of CD235a surface marker expression of sh83 and/or sh84 shRNA treated cells versus controls. FIG. 3B shows a representative plot of CD71 surface marker expression of sh83 and/or sh84 shRNA treated cells versus controls. More cells acquire CD235a and lose CD71 at earlier stages in the SH2B3 knockdown (sh83 and sh84 SH2B3 shRNA) cultures than in the shLuc control.

FIG. 4 shows representative flow cytometry plots showing cells stained with CD235a and Hoechst 33342 on day 18 of differentiation, and shows that at the late stages of the culture (day 16-18) more cells underwent enucleation (shown in the box), which is typical of mature RBCs. Numbers represent the mean percentage of CD235a+/Hoechst 33342-cells, indicative of enucleated RBCs, within the depicted gate±SD.

FIG. 5 shows representative cytocentrifuge images of May-Grünwald-Giemsa stained control and SH2B3-KD (sh83 and sh84 SH2B3 shRNA treated) cells at the indicated days of differentiation. The results shows that the cells matured at a morphological level faster and acquired more hemoglobin with reduced SH2B3 levels. A scale bar is shown in upper left panel.

FIG. 6 shows the mean amplification of control or SH2B3 KD peripheral blood-mobilized CD34+ HSPCs, showing that, in addition to the more robust erythroid differentiation, the SH2B3-KD cells (using sh83 and sh84 SH2B3 shRNA) expanded more than controls. Values shown are mean±standard error of the mean (n=3 independent experiments representative of >3 experiments using different donors).

FIG. 7 show improved expansion of Erythroid Cells with SH2B3 Suppression in Hematopoietic Stem Cells. FIG. 7 show mean yield of enucleated RBCs (CD235a+/Hoechst 33342-) observed for control and SH2B3 KD (sh83 and sh84 shRNA treated) samples derived from adult mobilized peripheral blood, showing that there was a 3-5 fold increase in overall yields with reduced SH2B3 levels when accounting for the yield of enucleated RBCs. Comparisons were done by a two-tailed Student's t test (n=3 independent experiments; *p<0.05; **p<0.01, ***p<0.001).

FIGS. 8A-8C show improved expansion of Erythroid Cells with SH2B3 Suppression in Hematopoietic Stem Cells. FIG. 8A shows the signal reduction of the fluorescent membrane dye PKH observed for control and SH2B3-KD adult HSPC derived erythroid cells. Ratios of PKH signal on day 9 compared with day 5, as determined by flow cytometry, are shown. Values are mean±the standard deviation and were normalized to the control. The comparison was performed by a two-tailed Student's t-test (n =3; **P<0.01; ***P <0.001). FIG. 8B shows absolute number of cell divisions based on PKH signal reduction observed for adult HPSC-derived erythroid cells between days 5 and 9 of culture. Values represent mean±the standard deviation. Comparisons were performed with a two-tailed Student's t-test (n=3; *P<0.05; **P<0.01, ***P<0.001). FIG. 8C shows an algorithm to calculate the PKH signal reduction and cell division.

FIG. 9 shows representative histogram plots showing eosin-5-maleimide signal on day 18 of mature RBCs derived from peripheral-blood-mobilized CD34+ cells, showing that the cells had similar eosin-5-maleimide fluorescence level as control cells, indicating that they matured similarly to control cells.

FIG. 10 is a schematic of adherent differentiation. CRISPR/Cas9 was used to create SH2B3 knockout (KO) human embryonic stem cell (hESC) lines. Isogenic controls were isolated. These cells were induced to undergo hematopoietic differentiation. Once hematopoietic progenitor cells were obtained using this method, they were then differentiated toward the erythroid lineage as shown in the bottom of the figure.

FIG. 11 shows that the SH2B3 KO hESCs differentiated as well or better than controls. SH2B3 KO (generataed using the CRISPR/Cas9 system) and isogenic control are shown with staining for erythroid surface phenotypic markers, CD71 and CD235a.

FIGS. 12A-12B show improved expansion of Erythroid Cells with SH2B3 Suppression in Hematopoietic and Pluripotent Stem Cells. FIGS. 12A and 12B show erythroid cell expansion of HPCs derived from two independent pairs of isogenic hESC clones; SH2B3-knockout BB5 line (KO BB5) (FIG. 12A) and SH2B3-knockout DB3 (KO DB3) (FIG. 12B), as compared to their wild-type isogenic controls (WT), showing that the cells with SH2B3 KO with CRISPR/Cas9 expanded to a considerably greater extent than controls. Thus, SH2B3 KO with CRISPR/Cas9 shows consistent increased expansion using a different KO clones (e.g., KO BB5 and KO DB3 clones) as compared to their respective isogenic controls (e.g, WT BB5 and WT DB3, respectively). The total cell numbers starting with 50,000 cells are shown at various time points as the mean±SD. Comparisons were performed by a two-tailed Student's t test (n=3; *p <0.05; **p<0.01; ***p<0.001; ****p<0.0001).

FIGS. 13A-13C shows in vitro Suppression of SH2B3 in primary HSPCs. FIG. 13A shows a simplified scheme illustrating differentiation of human CD34+ HSPCs into mature RBCs. FIG. 13B shows a scatter plot of mean gene expression values of control (Luc-KD for cells treated with control shLuc) and SH2B3-KD samples (n=3 independent samples for shLuc, sh83 shRNA, and sh84 shRNA). The coefficient of determination (r) is shown. FIG. 13C shows enrichment profiles from gene set enrichment analysis comparing the relative expression of genes in SH2B3-KD samples versus control. An enrichment plot showing an erythroid differentiation signature derived by comparing early erythroid progenitors with more differentiated cells is shown (p<0.0001 using a modified Kolmogorov-Smirnov statistical test).

FIGS. 14A-14E show improved expansion of Erythroid Cells with SH2B3 Suppression in Hematopoietic and Pluripotent Stem Cells. FIG. 14A shows the mean yield of enucleated RBCs (CD235a+/Hoechst 33342-) observed for control and SH2B3 KD (sh83 and sh84 shRNA) samples derived from cord blood HSPCs. Values shown are mean±SD and were normalized to the control. Comparisons were done by a two-tailed Student's t test (n=3 independent experiments; *p<0.05; **p<0.01, ***p<0.001). FIG. 14B shows the mean yield of enucleated RBCs (CD235a+/Hoechst 33342-) observed for control and SH2B3 KD samples derived from adult mobilized peripheral blood progenitors using a more efficient differentiation protocol involving a CD34+ expansion phase. Comparisons were done by a two-tailed Student's t test (n=4; ***p<0.001). FIG. 14C shows a simplified schematic illustrating differentiation of hESCs into RBCs. The hESCs were cultured in sequential cytokine combinations to induce the production of multipotent hematopoietic progenitor cells (HPCs) that were released into the medium around day 8. The HPCs were collected and cultured further in medium with EPO and SCF to support erythroid differentiation. FIG. 14D shows representative cytospin images of May-Grünwald-Giemsa stained control and SH2B3-KO erythroblasts at the indicated days of culture subsequent to HPC selection. FIG. 14E shows representative flow cytometry plots depicting the expression of CD71 and CD235a on erythroblasts derived from SH2B3-KO and isogenic WT hESCs.

FIGS. 15A-15E shows the characterization of Erythroid Cells from HSPCs Following SH2B3 Suppression (related to FIG. 14). FIG. 15A shows relative expansion of adult HSPCs observed for the indicated EPO concentrations. Erythroid expansion is calculated as total cell expansion until day 18 corrected for the percentage of CD235a positive cells observed by flow cytometry analysis on day 18. Values shown are mean±the standard deviation and are normalized to the expansion observed at 1 U/mL (n=3 per concentration). FIG. 15B shows bar graphs showing the frequency of annexin V+ apoptotic or dead control and SH2B3-KD samples on the indicated days of erythroid differentiation of adult HSPCs. Values show mean percentages±the standard deviation. A two-tailed Student's t-test was performed (n=3, n.s.=non-significant). FIG. 15C shows Western blots showing activation of phosphorylated STAT5 (pSTAT5) and phosphorylated KIT Y568 (pKIT Y568) in the human TF-1 erythroid cell line transduced with control (shLuc) or SH2B3 (sh84) shRNAs following cytokine starvation and stimulation with EPO and SCF. Cells were selected in puromycin for 48 hours before cytokine starvation. pKIT Y568 can be completely eliminated with serum starvation of cells, suggesting baseline stimulation with serum alone. STAT5 activation only occurs downstream of specific cytokine receptors, such as the EPO receptor. FIG. 15D shows expression of EPO responsive genes CISH and PIM1 with SH2B3 suppression in TF-1 erythroid cells undergoing overnight cytokine starvation or following 2 hours of stimulation with EPO. Values represent the mean±the standard deviation. Comparisons were performed with a two-tailed Student's t-test (n=3; *P<0.05; **P<0.01; ***P<0.001). FIG. 15E shows relative expansion of adult HSPCs corrected for CD235a positive cells at varying EPO and SCF concentrations as shown. Cultures were done using the more efficient differentiation protocol discussed in the Methods involving an expansion step prior to differentiation in the three-phase system, where EPO and SCF concentrations were varied in all phases. Values shown are the mean±the standard error of the mean. Comparisons were performed with a two-tailed Student's t-test (n=3; **P<0.01; ***P<0.001).

FIGS. 16A-16H show the characterization of Erythroid Cells from SH2B3 knock-down HSPCs and Pluripotent Stem Cells (FIG. 16A-16H is related to FIG. 14A-14E). FIG. 16A shows representative histogram plots showing Rh blood antigen expression on day 18 mature RBCs derived from adult CD3430 cells. FIG. 16B shows pyruvate kinase activity observed for control and SH2B3-KD mature RBCs generated from adult CD34+ cells at the indicated time points. Values shown are the mean±the standard deviation (n=2 independent experiments). FIG. 16C shows Coomassie blue stained SDS-PAGE gels showing major membrane protein bands from RBC ghosts derived from adult RBCs. FIG. 16D shows hemoglobin high performance liquid chromatography analysis of mature hemoglobin subtypes in hemolysates from the RBCs produced in culture. The peaks for HbF and HbA2 are shaded in the chromatograms with labels above the corresponding peaks. FIG. 16E is a schematic illustration showing the single guide RNA (gRNA) utilized to target the depicted SH2B3 DNA sequence adjacent to the protospacer adjacent motif (PAM) to cause double-strand breaks (DSB) by the Cas9 protein in exon 3 of the SH2B3 gene. FIG. 16F shows sequences for CRISPR/Cas9-targeted hESCs. The PAM sequence is highlighted in BOLD, and the predicted cleavage site is three bases upstream of the PAM. The allele sequences of one homozygous clone DB3 and two compound heterozygous clones BB5 and BF4 that harbor frameshift mutations in SH2B3 are shown. Note that the sense DNA sequence is shown. FIG. 16G shows the results of cell expansion of hESC-derived HPCs with SH2B3-KO and an isogenic control. HPCs were collected 9 days after initiation of differentiation for this experiment. The total cell number is shown as the mean±the standard deviation at the various time points. Comparisons were performed with a two-tailed Student's t-test at each indicated day of culture (n=3; NS=non-significant; *P<0.05; **P<0.01). FIG. 16H shows globin gene expression in hESC-derived erythroid cells (with either SH2B3 KO or WT alleles) on day 10 of HPC differentiation, as measured by quantitative RT-PCR. Expression relative to β-actin is shown on a log10 scale (n=3 per group).

### DETAILED DESCRIPTION

The present invention is directed to methods, systems, and compositions, and kits to produce red blood cells (RBCs) with increased quantity and quality from sources such as stem cells and/or progenitor cells. In particular, SH2B3 has been identified herein as an important negative regulator of RBC production from these sources. That is, the inventor has discovered that SH2B3 inhibits production of RBCs from stem cells and/or progenitor cells. As demonstrated herein, the inventor has discovered that lowering SH2B3 protein level and/or mRNA level and/or removing the SH2B3 gene using gene editing approaches (e.g., using a CRISPR/Cas9 or CRISPR/Cpfl) from the genome of the source stem cell or progenitor cell permit the generation of RBCs with increased quantity and quality from these sources.

Thus, the present invention releates to the inhibition of SH2B3 in methods, compositions and kits to produce, and improve yields of RBCs produced from stem cells and/or progenitor cells, e.g., as a replacement product for standard transfusions. Accordingly, aspects and embodiments of the invention relate to methods and compositions to inhibit SH2B3 in stem cells and/or progenitor cells for RBC production and methods of use thereof. It has been previously reported in WO2013/019857 that inhibition of AhR, Prox1 and/or SH2B3 increases the expansion of multipotent hematopoietic cells (MHCs). However, in contrast to the present invention, WO2013/019857 does not describe the differentiation of MHCs into RBCs or the collection of RBCs. And thus WO2013/019857 does not describe nor demonstrate that inhibition of SH2B3 in stem cells and/or progenitor cells can increase the quantity and/or quality of RBCs differentiated from these cells.

In one aspect, the invention provides a method of producing red blood cells (RBCs) ex vivo from a population of stem cells and/or progenitor cells, the method comprising: (i) inhibiting SH2B3 in the population of stem cells and/or progenitor cells; (ii) culturing the population of stem cells and/or progenitor cells for a time sufficient to induce differentiation of at least one stem cell or progenitor cell to a RBC; and (iii) collecting a population of RBCs.

Described herein are compositions and methods that reduce expression of SH2B3 mRNA or protein in a cell or in a mammal. Also described are compositions and methods for promoting RBC production and/or expansion from stem cells and/or progenitor cells ex vivo, thus permitting an increase in the amount of RBC for blood transfusions and/or blood banks available to patients as well as increasing the efficacy and/or potential of the same transplant material. In some embodiments, the compositions and methods allow increased yield of RBC from stem cells and/or progenitor cells ex vivo, in comparison to stem cells and/or progenitor cells not treated with an inhibitor of SH2B3, or not having the SH2B3 gene knocked out (i.e., using gene editing procedures as disclosed herein). In some embodiments, the compositions and methods allow increased yield of RBC from stem cells and/or progenitor cells obtained from a donor subject who has a blood Type O− (O, Rh−), thereby allowing increase of an unlimited supply of RBC from a universal donor subject. Also contemplated is administration of such iRNA compositions to enhance RBC expansion in vivo.

### Definitions

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Further, unless otherwise required by context, singular terms shall include pluralities and plural terms shall include the singular.

As used herein, the term “SH2B3” refers to either the SH2B3 protein or a nucleic acid encoding the SH2B3 protein, depending on the specific context. SH2B3 (also known as SH2B adaptor protein 3 or Lnk; NCBI Gene ID: 10019) is a member of intracellular adaptor protein family. The mRNA of SH2B3 for homo sapiens is known to have at least two isoforms (NCBI Accession No: NM_005475, NM_001291424). SH2B3 is expressed primarily in lymphocytes and hematopoietic precursor cells and regulates early lymphohematopoiesis.

As used herein, the term “ex vivo” refers to a situation or condition where biological cells obtained from an organism (e.g., a mammal) are cultured outside the organism.

The term “stem cell” as used herein, refers to an undifferentiated cell which is capable of proliferation and giving rise to more progenitor cells having the ability to generate a large number of mother cells that can in turn give rise to differentiated, or differentiable daughter cells. The daughter cells themselves can be induced to proliferate and produce progeny that subsequently differentiate into one or more mature cell types, while also retaining one or more cells with parental developmental potential.

Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Formally, it is possible that cells that begin as stem cells might proceed toward a more differentiated phenotype, but then “reverse” and re-express a less differentiated phenotype, i.e., a stem cell or stem-cell like phenotype, a term often referred to as “dedifferentiation” or “reprogramming” or “retrodifferentiation” by persons of ordinary skill in the art.

The term “progenitor” or “precursor” cell are used interchangeably herein and refer to cells that have a cellular phenotype that is more primitive (i.e., is at an earlier step along a developmental pathway than a fully differentiated cell) relative to a cell which it can give rise to by differentiation. Often, progenitor cells also have significant or very high proliferative potential. Progenitor cells can give rise to multiple distinct differentiated cell types or to a single differentiated cell type, depending on the developmental pathway and on the environment in which the cells develop and differentiate.

The term “pluripotent” as used herein refers to a cell with the capacity, under different conditions, to differentiate to more than one differentiated cell type, and preferably to differentiate to cell types characteristic of all three germ cell layers. Pluripotent cells are characterized primarily by their ability to differentiate to more than one cell type, preferably to all three germ layers, using, for example, a nude mouse teratoma formation assay. Pluripotency is also evidenced by the expression of embryonic stem (ES) cell markers, although the preferred test for pluripotency is the demonstration of the capacity to differentiate into cells of each of the three germ layers. It should be noted that simply culturing such cells does not, on its own, render them pluripotent. Reprogrammed pluripotent cells (e.g. induced pluripotent stem (iPS) cells as that term is defined herein) also have the characteristic of the capacity of extended passaging without loss of growth potential, relative to primary cell parents, which generally have capacity for only a limited number of divisions in culture.

The terms “embryonic stem cell” or “ES cell” are used interchangeably to refer to the pluripotent stem cells of the inner cell mass of the embryonic blastocyst (see U.S. Pat. Nos. 5,843,780, 6,200,806). Such cells can similarly be obtained from the inner cell mass of blastocysts derived from somatic cell nuclear transfer (see, for example, U.S. Pat. Nos. 5,945,577, 5,994,619, 6,235,970). The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The term “hematopoietic stem cells” or “HSCs” as used herein refers to pluripotent stem cells or multipotent stem cells or lymphoid or myeloid (derived from bone marrow) stem cells that can either differentiate into a progenitor cell of a lymphoid, erythroid or myeloid cell lineage or proliferate as a stem cell population without further differentiation having been initiated. HSCs can be isolated from bone marrow, peripheral blood, umbilical cord blood, or embryonic stem cells. HSCs can form cells such as erythrocytes (red blood cells), platelets, granulocytes (such as neutrophils, basophils, and eosinophils), macrophages, B-lymphocytes, T-lymphocytes, and Natural killer cells. HSC are capable of self-renewal or remaining a stem cell after cell division. HSCs are also capable of differentiation or starting a path to becoming a mature hematopoietic cell. HSCs can also be regulated in their mobility or migration or can be regulated by apoptosis or programmed cell death.

The terms “hematopoietic progenitor cells” or “HPCs” as used herein refer to primitive hematopoietic cells that have differentiated to a developmental stage that, when the cells are further exposed to an appropriate cytokine or a group of cytokines, they will differentiate further along the hematopoietic cell lineage. In contrast to HSCs, hematopoietic progenitor cells are only capable of limited self-renewal. “Hematopoietic progenitor cells” as used herein also include “precursor cells” that are derived from differentiation of hematopoietic progenitor cells and are the immediate precursors of mature differentiated hematopoietic cells. The term “hematopoietic progenitor cells”, as used herein include, but are not limited to, granulocyte-macrophage colony-forming cell (GM-CFC), megakaryocyte colony-forming cell (Mk-CFC), burst-forming unit erythroid (BFU-E), B cell colony-forming cell (B-CFC) and T cell colony-forming cell (T-CFC). “Precursor cells” include, but are not limited to, colony-forming unit-erythroid (CFU-E), granulocyte colony forming cell (G-CFC), colony-forming cell-basophil (CFC-Bas), colonyforming cell-eosinophil (CFC-Eo) and macrophage colonyforming cell (M-CFC) cells.

The terms “hematopoietic stem and progenitor cells” or “HSPCs” as used herein refer to a mixture of hematopoietic stem cells and hematopoietic progenitor cells.

As used herein, the terms “expanding” and “expansion” refer to substantially differentiation-less cell growth, i.e., increase of a cell population without differentiation accompanying such increase.

In the context of cell ontogeny, the adjective “differentiated”, or “differentiating” is a relative term, where a “differentiated cell” is a cell that has progressed further down the developmental pathway than the cell it is being compared with. Thus, stem cells can differentiate to lineage-restricted precursor cells (such as a hematopoietic stem cell), which in turn can differentiate into other types of precursor cells further down the pathway (such as a thymocyte, or a T lymphocyte precursor), and then to an end-stage differentiated cell, which plays a characteristic role in a certain tissue type, and may or may not retain the capacity to proliferate further.

The term “cell culture medium” (also referred to herein as a “culture medium” or “medium”) as referred to herein is a medium for culturing cells containing nutrients that maintain cell viability and support proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

The terms “increased,” “increase,” “enhance,” or “expand” are all used herein to generally mean an increase in the number of cells (e.g., stem cells, progenitor cells, or RBCs) or in the quality of cells by a statically significant amount; for the avoidance of any doubt, the terms “increased,” “increase,” “expand,” “expanded,” or “enhance” mean an increase, as compared to a reference level, of at least about 10%, of at least about 15%, of at least about 20%, of at least about 25%, of at least about 30%, of at least about 35%, of at least about 40%, of at least about 45%, of at least about 50%, of at least about 55%, of at least about 60%, of at least about 65%, of at least about 70%, of at least about 75%, of at least about 80%, of at least about 85%, of at least about 90%, of at least about 95%, or up to and including a 100%, or at least about a 2-fold, or at least about a 3-fold, or at least about a 4-fold, or at least about a 5-fold, at least about a 6-fold, or at least about a 7-fold, or at least about a 8-fold, at least about a 9-fold, at least about a 10-fold increase, at least about a 25-fold increase, at least about a 50-fold increase, at least about a 100-fold increase, or any increase of 100-fold or greater, as compared to a control or reference level. A control sample or control level is used herein to describe a population of stem cells and/or progenitor cells obtained from the same biological source that has, for example, normal or undisrupted SH2B3 gene function.

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” typically means a decrease by at least about 5%-10% as compared to a reference level, for example a decrease by at least about 20%, or at least about 30%, or at least about 40%, or at least about 50%, or at least about 60%, or at least about 70%, or at least about 80%, or at least about 90% decrease (i.e. absent level as compared to a reference sample), or any decrease between 10-100% as compared to a reference level.

The term “statistically significant” or “significantly” refers to statistical significance and generally means a two standard deviation (2SD) or greater difference.

As used herein, the term “significant” or “significantly” should be interpreted as if modified by the term “statistically”.

As used herein, the term “nucleic acid”, “polynucleotide” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In some embodiments, the nucleic acid can be DNA. In some embodiments, the nucleic acid can be RNA. Suitable nucleic acid molecules are DNA, including genomic DNA or cDNA. Other suitable nucleic acid molecules are RNA, including mRNA.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site comprising the complementarity determining regions (CDRs) of an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds to a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibody (dAb) fragments (see, e.g. de Wildt et al., Eur J. Immunol 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized. In certain embodiments, specific binding is indicated by a dissociation constant on the order of ≦10⁻⁸ M, ≦10⁻⁹ M, ≦10⁻¹⁹ M or below.

As used herein, “expression level” refers to the number of mRNA molecules and/or polypeptide molecules encoded by a given gene that are present in a cell or sample. Expression levels can be increased or decreased relative to a reference level.

As used herein, the term “iRNA agent” or “RNAi agent” refers to an agent that contains RNA as that term is defined herein, and which mediates the targeted cleavage of an RNA transcript via an RNA-induced silencing complex (RISC) pathway. In one embodiment, an iRNA as described herein inhibits the expression SH2B3/Lnk a stem cell or progenitor cell, e.g., HSC or a mammal.

As used herein, “target sequence” refers to a contiguous portion of the nucleotide sequence of a messenger RNA (mRNA) molecule formed during the transcription of a gene, including mRNA that is a product of RNA processing of a primary transcription product. The target portion of the sequence will be at least long enough to serve as a specific binding site for an iRNA agent and/or as a substrate for iRNA-directed cleavage at or near that portion. For example, the target sequence will generally be from 9-36 nucleotides in length, e.g., 15-30 nucleotides in length, including all sub-ranges therebetween. As non-limiting examples, the target sequence can be from 15-30 nucleotides, 15-26 nucleotides, 15-23 nucleotides, 15-22 nucleotides, 15-21 nucleotides, 15-20 nucleotides, 15-19 nucleotides, 15-18 nucleotides, 15-17 nucleotides, 18-30 nucleotides, 18-26 nucleotides, 18-23 nucleotides, 18-22 nucleotides, 18-21 nucleotides, 18-20 nucleotides, 19-30 nucleotides, 19-26 nucleotides, 19-23 nucleotides, 19-22 nucleotides, 19-21 nucleotides, 19-20 nucleotides, 20-30 nucleotides, 20-26 nucleotides, 20-25 nucleotides, 20-24 nucleotides,20-23 nucleotides, 20-22 nucleotides, 20-21 nucleotides, 21-30 nucleotides, 21-26 nucleotides, 21-25 nucleotides, 21-24 nucleotides, 21-23 nucleotides, or 21-22 nucleotides.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions can include: 400 mM NaC1, 40 mM PIPES pH 6.4, 1 mM EDTA, 50° C. or 70° C. for 12-16 hours followed by washing. Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides.

Complementary sequences within an iRNA, e.g., within a dsRNA as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

“Complementary” sequences, as used herein, can also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but are not limited to, G:U Wobble or Hoogstein base pairing.

The terms “complementary,” “fully complementary” and “substantially complementary” herein can be used with respect to the base matching between the sense strand and the antisense strand of a dsRNA, or between the antisense strand of an iRNA agent and a target sequence, as will be understood from the context of their use.

As used herein, a polynucleotide that is “substantially complementary to at least part of” a messenger RNA (mRNA) refers to a polynucleotide that is substantially complementary to a contiguous portion of the mRNA of interest (e.g., an mRNA encoding SH2B3). For example, a polynucleotide is complementary to at least a part of a mRNA if the sequence is substantially complementary to a non-interrupted portion of the mRNA.

The term “double-stranded RNA” or “dsRNA,” as used herein, refers to an iRNA that includes an RNA molecule or complex of molecules having a hybridized duplex region that comprises two anti-parallel and substantially complementary nucleic acid strands, which will be referred to as having “sense” and “antisense” orientations with respect to a target RNA. The duplex region can be of any length that permits specific degradation of a desired target RNA through a RISC pathway, but will typically range from 9 to 36 base pairs in length, e.g., 15-30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range therein between, including, but not limited to 15-30 base pairs, 15-26 base pairs, 15-23 base pairs, 15-22 base pairs, 15-21 base pairs, 15-20 base pairs, 15-19 base pairs, 15-18 base pairs, 15-17 base pairs, 18-30 base pairs, 18-26 base pairs, 18-23 base pairs, 18-22 base pairs, 18-21 base pairs, 18-20 base pairs, 19-30 base pairs, 19-26 base pairs, 19-23 base pairs, 19-22 base pairs, 19-21 base pairs, 19-20 base pairs, 20-30 base pairs, 20-26 base pairs, 20-25 base pairs, 20-24 base pairs, 20-23 base pairs, 20-22 base pairs, 20-21 base pairs, 21-30 base pairs, 21-26 base pairs, 21-25 base pairs, 21-24 base pairs, 21-23 base pairs, or 21-22 base pairs. dsRNAs generated in the cell by processing with Dicer and similar enzymes are generally in the range of 19-22 base pairs in length. One strand of the duplex region of a dsDNA comprises a sequence that is substantially complementary to a region of a target RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (herein referred to as a “hairpin loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of a dsRNA are comprised by separate RNA molecules, those molecules need not, but can be covalently connected. Where the two strands are connected covalently by means other than a hairpin loop, the connecting structure is referred to as a “linker.” The term “siRNA” is also used herein to refer to a dsRNA as described above.

The skilled artisan will recognize that the term “RNA molecule” or “ribonucleic acid molecule” encompasses not only RNA molecules as expressed or found in nature, but also analogs and derivatives of RNA comprising one or more ribonucleotide/ribonucleoside analogs or derivatives as described herein or as known in the art. Strictly speaking, a “ribonucleoside” includes a nucleoside base and a ribose sugar, and a “ribonucleotide” is a ribonucleoside with one, two or three phosphate moieties. However, the terms “ribonucleoside” and “ribonucleotide” can be considered to be equivalent as used herein. The RNA can be modified in the nucleobase structure or in the ribose-phosphate backbone structure, e.g., as described herein below. However, the molecules comprising ribonucleoside analogs or derivatives must retain the ability to form a duplex. As non-limiting examples, an RNA molecule can also include at least one modified ribonucleoside including but not limited to a 2′-O-methyl modified nucleoside, a nucleoside comprising a 5′ phosphorothioate group, a terminal nucleoside linked to a cholesteryl derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. Alternatively, an RNA molecule can comprise at least two modified ribonucleosides, at least 3, at least 4, at least 5, at least 6, at least 7, at least 8, at least 9, at least 10, at least 15, at least 20 or more, up to the entire length of the dsRNA molecule. The modifications need not be the same for each of such a plurality of modified ribonucleosides in an RNA molecule. In one embodiment, modified RNAs contemplated for use in methods and compositions described herein are peptide nucleic acids (PNAs) that have the ability to form the required duplex structure and that permit or mediate the specific degradation of a target RNA via a RISC pathway.

In one aspect, a modified ribonucleoside includes a deoxyribonucleoside. In such an instance, an iRNA agent can comprise one or more deoxynucleosides, including, for example, a deoxynucleoside overhang(s), or one or more deoxynucleosides within the double stranded portion of a dsRNA. However, it is self evident that under no circumstances is a double stranded DNA molecule encompassed by the term “IRNA.”

In one aspect, an RNA interference agent includes a single stranded RNA that interacts with a target RNA sequence to direct the cleavage of the target RNA. Without wishing to be bound by theory, long double stranded RNA introduced into plants and invertebrate cells is broken down into siRNA by a Type III endonuclease known as Dicer (Sharp et al., Genes Dev. 2001, 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleaves the target to induce silencing (Elbashir, et al., (2001) Genes Dev. 15:188). Thus, in one aspect the technology described herein relates to a single stranded RNA that promotes the formation of a RISC complex to effect silencing of the target gene.

As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) can be on the sense strand, the antisense strand or any combination thereof. Furthermore, the nucleotide(s) of an overhang can be present on the 5′ end, 3′ end or both ends of either an antisense or sense strand of a dsRNA.

In one embodiment, the antisense strand of a dsRNA has a 1-10 nucleotide overhang at the 3′ end and/or the 5′ end. In one embodiment, the sense strand of a dsRNA has a 1-10 nucleotide overhang at the 3′ end and/or the 5′ end. In another embodiment, one or more of the nucleotides in the overhang is replaced with a nucleoside thiophosphate.

The terms “blunt” or “blunt ended” as used herein in reference to a dsRNA or dsDNA mean that there are no unpaired nucleotides or nucleotide analogs at a given terminal end of a dsRNA or dsDNA molecule, i.e., no nucleotide overhang. One or both ends of a dsRNA or dsDNA can be blunt. Where both ends of a dsRNA or dsDNA are blunt, the dsRNA or dsDNAis said to be blunt ended. To be clear, a “blunt ended” dsRNA or dsDNA is a dsRNA or dsDNAthat is blunt at both ends, i.e., no nucleotide overhang at either end of the molecule. Most often such a molecule will be double-stranded over its entire length. In contrast “sticky ends” refers to dsDNA or dsRNA molecule that has at least 1 or more (typically 2-5 or more) nucleocleotide overhang.

The term “antisense strand” or “guide strand” refers to the strand of an iRNA, e.g., a dsRNA, which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′ and/or 3′ terminus.

The term “sense strand,” or “passenger strand” as used herein, refers to the strand of an iRNA that includes a region that is substantially complementary to a region of the antisense strand as that term is defined herein.

As used herein, in one embodiment, the term “SNALP” refers to a stable nucleic acid-lipid particle. A SNALP represents a vesicle of lipids coating a reduced aqueous interior comprising a nucleic acid such as an iRNA or a plasmid from which an iRNA is transcribed. SNALPs are described, e.g., in U.S. Patent Application Publication Nos. 20060240093, 20070135372, and in International Application No. WO 2009082817. These applications are incorporated herein by reference in their entirety. Examples of “SNALP” formulations are described elsewhere herein.

As used herein, the phrase “inhibit the expression of,” refers to at an least partial reduction of gene expression of a gene encoding SH2B3 in a cell treated with SH2B3 inhibitor (e.g., an iRNA composition as described herein) compared to the expression of SH2B3 in an untreated cell.

The terms “silence,” “inhibit the expression of,” “down-regulate the expression of,” “suppress the expression of,” and the like, in so far as they refer to SH2B3, herein refer to the at least partial suppression of the expression of a gene encoding SH2B3, as manifested by a reduction of the amount of mRNA encoding SH2B3 which can be isolated from or detected in a first cell or group of cells in which that gene is transcribed and which has or have been treated such that the expression of SH2B3 is inhibited, as compared to a second cell or group of cells substantially identical to the first cell or group of cells but which has or have not been so treated (control cells). The degree of inhibition is usually expressed in terms of

$$( \frac{[ mRNA in control cells ] - [ mRNA in treated cells ]}{[ mRNA in control cells ]} ) \times 100 \%$$

Alternatively, the degree of inhibition can be given in terms of a reduction of a parameter that is functionally linked to gene expression, e.g., the amount of protein encoded by a gene, or the number of cells displaying a certain phenotype. In principle, gene silencing can be determined in any cell expressing, either constitutively or by genomic engineering, and by any appropriate assay. However, when a reference is needed in order to determine whether a given iRNA (or gene editing procedure) inhibits the expression of the gene encoding SH2B3 by a certain degree and therefore is encompassed by the technology described herein, the assays provided in the Examples below shall serve as such reference.

For example, in certain instances, expression of SH2B3 is suppressed by at least about 10%, 15%, 20%, 25%, 30%, 35%, 40%, 45%, or 50% by administration of an iRNA featured herein. In some embodiments, a gene encoding SH2B3 in a cell is suppressed by at least about 60%, 70%, or 80% or more than 80% by administration of an iRNA or gene editing proceedures (i.e., CRISPR/Cas9 or CRISPR/Cpf1) as featured herein. In some embodiments, a gene encoding SH2B3 is suppressed by at least about 85%, 90%, 95%, 98%, 99% or more by administration of an iRNA (or gene editing proceedures) as described herein.

“Introducing into a cell,” when referring to an iRNA, means facilitating or effecting uptake or absorption into the cell, as is understood by those skilled in the art. Absorption or uptake of an iRNA can occur through unaided diffusive or active cellular processes, or by auxiliary agents or devices. The meaning of this term is not limited to cells in vitro; an iRNA can also be “introduced into a cell,” wherein the cell is part of a living organism. In such an instance, introduction into the cell will include the delivery to the organism. For example, for in vivo delivery, iRNA can be injected into a tissue site or administered systemically. In vivo delivery can also be by a beta-glucan delivery system, such as those described in U.S. Pat. Nos. 5,032,401 and 5,607,677, and U.S. Publication No. 2005/0281781 which are hereby incorporated by reference in their entirety. In vitro introduction into a cell includes methods known in the art such as electroporation and lipofection. Further approaches are described herein below or are known in the art.

The terms “subject” and “individual” are used interchangeably herein, and refer to an animal, for example, a human from whom cells for use in the methods described herein can be obtained (i.e., donor subject) and/or to whom treatment, including prophylactic treatment, with the cells as described herein, is provided, i.e., recipient subject. For treatment of those conditions or disease states that are specific for a specific animal such as a human subject, the term subject refers to that specific animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g. dog, cat, horse, and the like, or production mammal, e.g. cow, sheep, pig, and the like.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. The administration can be systemic or local.

As used herein, the term “effective amount” when used to describe an antagonist of SH2B3, refers to an amount sufficient to decrease the activity of the SH2B3 protein or decrease SH2B3 mRNA or protein level by at least 10%, e.g., at least 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, or at least about 90%.

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (4 ed.), Cold Spring Harbor Laboratory Press, Cold Spring Harbor, N.Y., USA (2012); Davis et al., Basic Methods in Molecular Biology, Elsevier Science Publishing, Inc., New York, USA (1995); or Methods in Enzymology: Guide to Molecular Cloning Techniques Vol.152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

As used herein the term “comprising” or “comprises” is used in reference to compositions, methods, and respective component(s) thereof, that are useful to an embodiment, yet open to the inclusion of unspecified elements, whether useful or not.

As used herein the term “consisting essentially of” refers to those elements required for a given embodiment. The term permits the presence of elements that do not materially affect the basic and novel or functional characteristic(s) of that embodiment of the invention.

Other than in the operating examples, or where otherwise indicated, all numbers expressing quantities of ingredients or reaction conditions used herein should be understood as modified in all instances by the term “about.” The term “about” when used in connection with percentages may mean ±1% of the value being referred to. For example, about 100 means from 99 to 101.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

### Pluripotent Stem Cells and Progenitor Cells

In some embodiments, the population of stem cells and/or progenitor cells can be hematopoietic stem cells, hematopoietic progenitor cells, pluripotent stem cells, induced pluripotent stem cells (iPSCs), embryonic stem cells, or combinations thereof.

In some embodiments, the population of stem cells and/or progenitor cells is of mammalian origin. In some embodiments, the population of stem cells and/or progenitor cells is of human origin.

In some embodiments, the stem cells and/or progenitor cells can be obtained from an autologous donor. That is, the donor of the stem cells or progenitor cells will also be the recipient of the RBCs derived from such stem cells and/or progenitor cells. In some embodiments, the stem cells and/or progenitor cells can be obtained from an allogenic donor, such as a sibling, parent, or other relative of a subject in need of blood transfusion. Examples of patients that can benefit from autologous and/or allogenic donation are well known in the art and include, without limitation, those suffering from an autoimmune disorder, blood disease or disorder, immune disease or disorder, or other related diseases or conditions. RBC compatibility is well known in the art and is summarized in Table 1.

TABLE 1
Red blood cell compatibility table

| | DONOR | | | | | | | |
|---|---|---|---|---|---|---|---|---|
| Recipient | O− | O+ | A− | A+ | B− | B+ | AB− | AB+ |
| O− | ✓ | x | x | x | x | x | x | x |
| O+ | ✓ | ✓ | x | x | x | x | x | x |
| A− | ✓ | x | ✓ | x | x | x | x | x |
| A+ | ✓ | ✓ | ✓ | ✓ | x | x | x | x |
| B− | ✓ | x | x | x | ✓ | x | x | x |
| B+ | ✓ | ✓ | x | x | ✓ | ✓ | x | x |
| AB− | ✓ | x | ✓ | x | ✓ | x | ✓ | x |
| AB+ | ✓ | ✓ | ✓ | ✓ | ✓ | ✓ | ✓ | ✓ |

In some embodiments, the population of stem cells is a population of hematopoietic stem cells. It is known in the art that HSCs may or may not include CD34+ cells. CD34+ cells are immature cells that express the CD34 cell surface marker. CD34+ cells are believed to include a subpopulation of cells with stem cell properties. HSCs include pluripotent stem cells, multipotent stem cells (e.g., a lymphoid stem cell), and/or stem cells committed to specific hematopoietic lineages. The stem cells committed to specific hematopoietic lineages can be of T cell lineage, B cell lineage, dendritic cell lineage, Langerhans cell lineage and/or lymphoid tissue-specific macrophage cell lineage. In addition, HSCs also refer to long term HSC (LT-HSC) and short term HSC (ST-HSC). A long term stem cell typically includes the long term (more than three months) contribution to multilineage engraftment after transplantation. A short term stem cell is typically anything that lasts shorter than three months, and/or that is not multilineage. LT-HSC and ST-HSC are distinguished, for example, based on their cell surface marker expression. LT-HSC are CD34−, SCA-1+, Thyl.1+/lo, C-kit+, Un-, CD135−, Slamfl/CD150+, whereas ST-HSC are CD34+, SCA-1+, Thyl.1+/lo, C-kit+, lin-, CD135−, Slamfl/CD150+, Mac-1 (CDlIb)lo (Handbook of Stem Cells. Lanza, R. P. et al. (Eds.) Elsevier Academic Press Burlington, Mass. (2004)). In addition, ST-HSC are less quiescent (i.e., more active) and more proliferative than LT-HSC. LT-HSC have unlimited self-renewal (i.e., they survive throughout adulthood), whereas ST-HSC have limited self-renewal (i.e., they survive for only a limited period of time). Any of these HSCs can be used advantageously in any of the methods described herein.

In some embodiments, hematopoietic stem cells (HSC) encompassed for use in the methods, compositions and kits as disclosed herein include primitive cells capable of differentiating to generate all cells of the hematopoietic lineage. In some embodiments, HSC, encompassed for use in the methods and compositions as disclosed herein also includes more generally to immature blood cells having the capacity to self-renew and to differentiate into more mature blood cells comprising granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and/or monocytes (e.g., monocytes, macrophages). During development, the site of hematopoiesis translocates from the fetal liver to the bone marrow, which then remains the site of hematopoiesis throughout adulthood.

In some embodiments, the stem cells or progenitor cells are obtained from a donor subject who is Type O blood type, and therefore is a universal donor. In some embodiments, the stem cells or progenitor cells are obtained from a donor subject who is O negative (O−) blood type (i.e., Type O, Rh factor negative), and therefore is a universal donor. In some embodiments, the stem cells or progenitor cells or HSCs are obtained from a subject whom is Type AB bloot type, and is a universal plasma donor. In some embodiments, the stem cells or progenitor cells are obtained from a subject of the same ethnic background as the recipient subject.

Table 2: shows the distribution of different blood types in different enthic backgrounds.

| Blood | | African | | |
|---|---|---|---|---|
| type | Caucasians | American | Hispanic | Asian |
| O+ | 37% | 47% | 53% | 39% |
| O− | 8% | 4% | 4% | 1% |
| A+ | 33% | 24% | 29% | 27% |
| A− | 7% | 2% | 2% | 0.5% |
| B+ | 9% | 18% | 9% | 25% |

HSCs can be obtained from blood products. A blood product includes a product obtained from the body or an organ of the body containing cells of hematopoietic origin. Such sources include unfractionated bone marrow, peripheral blood mononuclear cells, umbilical cord blood, umbilical cord tissue, peripheral blood (e.g., G-CSF mobilize peripheral blood), liver, thymus, lymph and spleen. All of the aforementioned crude or unfractionated blood products can be enriched for cells having hematopoietic stem cell characteristics in a number of ways. For example, the more mature, differentiated cells are selected against, via cell surface molecules they express. Optionally, the blood product is fractionated by selecting for CD34+ cells. CD34+ cells include a subpopulation of cells capable of self -renewal and pluripotentiality. Such selection is accomplished using, for example, commercially available magnetic anti-CD34 beads (Dynal, Lake Success, N.Y.). Unfractionated blood products are optionally obtained directly from a donor or retrieved from cryopreservative storage.

Sources for HSC expansion also include aorta-gonad-mesonephros (AGM) derived cells, embryonic stem cells (ESCs) and induced pluripotent stem cells (iPSCs). ESCs are well-known in the art, and can be obtained from commercial or academic sources (Thomson et al., 282 Sci. 1145-47 (1998)). iPSCs are a type of pluripotent stem cell artificially derived from a non-pluripotent cell, typically an adult somatic cell, by inducing a “forced” expression of certain genes (Baker, Nature Rep. Stem Cells (Dec. 6, 2007); Vogel & Holden, 23 Sci. 1224-25 (2007)). ESCs, AGM, and iPSCs can be derived from animal or human sources. The AGM stem cell is a cell that is born inside the aorta, and colonizes the fetal liver.

In some embodiments, the population of stem cells is a population of iPSCs. The term “induced pluripotent stem cell” or “iPSC” or “iPS cell” refers to a cell derived from reprogramming of the differentiation state of a differentiated cell (e.g. a somatic cell) into a pluripotent cell. An induced pluripotent stem cell a) can self-renew, and b) can differentiate to produce all types of cells in an organism. iPS cells have an ES cell-like morphology, growing as flat colonies with large nucleo-cytoplasmic ratios, defined borders and prominent nucleoli. In addition, iPS cells express one or more key pluripotency markers known by one of ordinary skill in the art, including but not limited to Alkaline Phosphatase, SSEA3, SSEA4, Sox2, Oct3/4, Nanog, T AI60, TRA I81 , TDGF 1 , Dnmt3b, FoxD3, GDF3, Cyp26al, TERT, and zfp42. IPS cells may be generated by providing the cell with “reprogramming factors”, i.e., one or more, e.g., a cocktail, of biologically active factors that act on a cell to alter transcription, thereby reprogramming a cell to pluripotency. Examples of methods of generating and characterizing iPS cells may be found in, for example, Application Nos. US20090047263, US20090068742, US20090191 159, US20090227032, US20090246875, and US20090304646, the disclosures of each of which are incorporated herein by reference.

In some embodiments, the population of stem cells is a population of nuclear-transfer stem cells (NT-ESCs), or human NT-ESCs (hNT-ESCs). Methods for production of pluripotent stem cells via somatic cell nuclear transfer (SCNT) are well known in the art, and include, methods disclosed in U.S. Pat. Nos. 5,843,780 and 6,200,806 as well as methods where ESC are obtained from the inner cell mass of blastocysts derived from somatic cell nuclear transfer are described in U.S. Pat. Nos. 5,945,577; 5,994,619; 6,235,970, which are incorporated herein in their entirety by reference. Furthermore, methods for increasing the efficiency of SCNT are disclosed in International application PCT/US15/50178, filed on Sep. 15, 2015 which is incorporated herein in its entirety by reference. The distinguishing characteristics of an embryonic stem cell define an embryonic stem cell phenotype. Accordingly, a cell has the phenotype of an embryonic stem cell if it possesses one or more of the unique characteristics of an embryonic stem cell such that that cell can be distinguished from other cells. Exemplary distinguishing embryonic stem cell characteristics include, without limitation, gene expression profile, proliferative capacity, differentiation capacity, karyotype, responsiveness to particular culture conditions, and the like.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

In some embodiments, the population of progenitor cells is a population of hematopoietic progenitor cells. Hematopoietic progenitor cells can be obtained from a variety of sources including, for example, bone marrow, peripheral blood, and umbilical cord blood. Hematopoietic progenitor cells can also be obtained from peripheral blood of a progenitor cell donor. Prior to harvest of the cells from peripheral blood, the donor can be treated with a cytokine, such as granulocyte-colony stimulating factor, to promote cell migration from the bone marrow to the blood compartment. Cells can be collected via an intravenous tube and filtered to isolate white blood cells for transplantation. The white blood cell population obtained (i.e., a mixture of stem cells, progenitors and white blood cells of various degrees of maturity) can be transplanted as a heterogeneous mixture or hematopoietic progenitor cells can further be isolated using cell surface markers known to those of skill in the art.

Hematopoietic progenitor cells, as the term is used herein, are capable of differentiation into one or more mature cell types of the hematopoietic lineage, but are not capable of long-term self-renewal. Thus, hematopoietic progenitor cells can restore and sustain hematopoiesis for three to four months (Marshak, D. R., et al. (2001). Stem cell biology, Cold Spring Harbor, N.Y.: Cold Spring Harbor Laboratory Press) and are important for recovery in the period immediately following a hematopoietic progenitor cell transplant in an individual. Hematopoietic progenitor cells useful for transplantation can be obtained from a variety of sources including, for example, bone marrow, peripheral blood, and umbilical cord blood.

Bone marrow can be obtained by puncturing bone with a needle and removing bone marrow cells with a syringe (herein called “bone marrow aspirate”). Hematopoietic progenitor cells can be isolated from the bone marrow aspirate prior to transplantation by using surface markers specific for hematopoietic progenitor cells, or alternatively whole bone marrow can be transplanted into an individual to be treated with the methods described herein.

Hematopoietic progenitor cells and/or a heterogeneous hematopoietic progenitor cell population can also be isolated from human umbilical cord and/or placental blood. Such blood can be collected by several methods known in the art. For example, because umbilical cord blood is a rich source of HSPCs (see Nakahata & Ogawa, 70 J. Clin. Invest. 1324-28 (1982); Prindull et al., 67 Acta. Paediatr. Scand. 413-16 (1978); Tchernia et al., 97(3) J. Lab. Clin. Med. 322-31 (1981)), an excellent source for neonatal blood is the umbilical cord and placenta. Prior to cryopreservation, the neonatal blood can be obtained by direct drainage from the cord and/or by needle aspiration from the delivered placenta at the root and at distended veins. See, e.g., U.S. Pat. No. 7,160,714; No. 5,114,672; No. 5,004,681; U.S. patent application Ser. No. 10/076,180, Pub. No. 20030032179. Indeed, umbilical cord blood stem cells have been used to reconstitute hematopoiesis in children with malignant and nonmalignant diseases after treatment with myeloablative doses of chemo-radiotherapy. Sirchia & Rebulla, 84 Haematologica 738-47 (1999). See also Laughlin 27 Bone Marrow Transplant. 1-6 (2001); U.S. Pat. No. 6,852,534. Additionally, it has been reported that stem and progenitor cells in cord blood appear to have a greater proliferative capacity in culture than those in adult bone marrow. Salahuddin et al., 58 Blood 931-38 (1981); Cappellini et al., 57 Brit. J. Haematol. 61-70 (1984).

Alternatively, fetal blood can be taken from the fetal circulation at the placental root with the use of a needle guided by ultrasound (Daffos et al., 153 Am. J. Obstet. Gynecol. 655-60 (1985); Daffos et al., 146 Am. J. Obstet. Gynecol. 985-87 (1983), by placentocentesis (Valenti, 115 Am. J. Obstet. Gynecol. 851-53 (1973); Cao et al., 19 J. Med. Genet. 81-87 (1982)), by fetoscopy (Rodeck, in Prenatal Diagnosis, (Rodeck & Nicolaides, eds., Royal College of Obstetricians & Gynaecologists, London, 1984)) and cryopreserved. Indeed, the chorionic villus and amniotic fluid, in addition to cord blood and placenta, are sources of pluripotent fetal stem cells (see WO 2003 042405).

Various kits and collection devices are known for the collection, processing, and storage of cord blood. See, e.g., U.S. Pat. No. 7,147,626; U.S. Pat. No. 7,131,958. Collections should be made under sterile conditions, and the blood can be treated with an anticoagulant. Such anticoagulants include, for example, citrate-phosphate-dextrose, acid citrate-dextrose, Alsever's solution (Alsever & Ainslie, 41 N.Y. St. J. Med. 126-35 (1941), DeGowin's Solution (DeGowin et al., 114 JAMA 850-55 (1940)), Edglugate-Mg (Smith et al., 38 J. Thorac. Cardiovasc. Surg. 573-85 (1959)), Rous-Turner Solution (Rous & Turner, 23 J. Exp. Med. 219-37 (1916)), other glucose mixtures, heparin, or ethyl biscoumacetate. See Hum, Storage of Blood 26-160 (Acad. Press, NY, 1968).

Various procedures are known in the art or described herein and can be used to enrich collected cord blood for HSPCs. These include but are not limited to equilibrium density centrifugation, velocity sedimentation at unit gravity, immune rosetting and immune adherence, counterflow centrifugal elutriation, T-lymphocyte depletion, and fluorescence-activated cell sorting, alone or in combination. See, e.g., U.S. Pat. No. 5,004,681.

In some embodiments, the HSPCs can be obtained from crypgenically prepared blood. In some embodiments, collected blood is prepared for cryogenic storage by addition of cryoprotective agents such as DMSO (Lovelock & Bishop, 183 Nature 1394-95 (1959); Ashwood-Smith 190 Nature 1204-05 (1961)), glycerol, polyvinylpyrrolidine (Rinfret, 85 Aim. N.Y. Acad. Sci. 576-94 (1960)), polyethylene glycol (Sloviter & Ravdin, 196 Nature 899-900 (1962)), albumin, dextran, sucrose, ethylene glycol, i-erythritol, D-ribitol, D-mannitol (Rowe, 3(1) Cryobiology 12-18 (1966)), D-sorbitol, i-inositol, D-lactose, choline chloride (Bender et al., 15J. Appl. Physiol. 520-24 (1960)), amino acids (Phan & Bender, 20 Exp. Cell Res. 651-54 (1960)), methanol, acetamide, glycerol monoacetate (Lovelock, 56 Biochem. J. 265-70 (1954)), and inorganic salts (Phan & Bender, 104 Proc. Soc. Exp. Biol. Med. (1960)). Addition of plasma (e.g., to a concentration of 20%-25%) can augment the protective effect of DMSO.

Collected blood should be cooled at a controlled rate for cryogenic storage. Different cryoprotective agents and different cell types have different optimal cooling rates. See e.g., Rapatz, 5 Cryobiology 18-25 (1968), Rowe & Rinfret, 20 Blood 636-37 (1962); Rowe, 3 Cryobiology 12-18 (1966); Lewis et al., 7 Transfusion 17-32 (1967); Mazur, 168 Science 939-49 (1970). Considerations and procedures for the manipulation, cryopreservation, and long-term storage of HSPCs sources are known in the art. See e.g., U.S. Pat. No. 4,199,022; U.S. Pat. No. 3,753,357; U.S. Pat. No. 4,559,298; U.S. Pat. No. 5,004,681. There are also various devices with associated protocols for the storage of blood. U.S. Pat. No. 6,226,997; U.S. Pat. No. 7,179,643

Considerations in the thawing and reconstitution of blood for HSPCs are also known in the art. U.S. Pat. No. 7,179,643; U.S. Pat. No. 5,004,681. The blood can also be treated to prevent clumping (see Spitzer, 45 Cancer 3075-85 (1980); Stiff et al., 20 Cryobiology 17-24 (1983), and to remove toxic cryoprotective agents (U.S. Pat. No. 5,004,681).

Methods to isolate stem cells and/or progenitor cells from a blood source are known in the art and are not described in detail here. For example, hematopoietic stem cells can be isolated using commercially available antibodies that bind to hematopoietic stem cell surface antigens, e.g. CD34, using methods known to those of skill in the art.

In general, cells as described herein can be maintained and expanded in culture medium that is available to and well-known in the art. Such media include, but are not limited to, Dulbecco's Modified Eagle's, F-12K™, EAGLE'S MINIMUM ESSENTIAL MEDIUM™ (DMEM), DMEM F12 Medium.™, and Serum-Free™, RPMI-1640 Medium™, Iscove's Modified Dulbecco's Medium™ (IMDM). Many media for culture and expansion of hematopoietic cells are also available as low-glucose formulations, with or without sodium pyruvate.

Also contemplated herein is supplementation of cell culture medium with mammalian sera. Sera often contain cellular factors and components that are necessary for viability and expansion. Examples of sera include fetal bovine serum (FBS), bovine serum (BS), calf serum (CS), fetal calf serum (FCS), newborn calf serum (NCS), goat serum (GS), horse serum (HS), human serum, chicken serum, porcine serum, sheep serum, rabbit serum, serum replacements and bovine embryonic fluid. It is understood that sera can be heat-inactivated at 55-65.degree. C. if deemed necessary to inactivate components of the complement cascade.

Additional supplements also can be used advantageously to supply the cells with the necessary trace elements for optimal growth and expansion. Such supplements include, for example, insulin, transferrin, sodium selenium and combinations thereof. These components can be included in a salt solution including, but not limited to, (HBSS), EARLE'S SALT™, Hanks' Balanced Salt Solution, antioxidant supplements, MCDB-201™. Solution saline (PBS), ascorbic acid and ascorbic acid-2-phosphate, as well as additional amino acids. Many cell culture media already contain amino acids, however, some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. It is well within the skill of one in the art to determine the proper concentrations of these supplements.

Hormones also can be advantageously used in the cell cultures described herein and include, but are not limited to, D-aldosterone, diethylstilbestrol (DES), dexamethasone, .beta.-estradiol, hydrocortisone, insulin, prolactin, progesterone, somatostatin/human growth hormone (HGH), thyrotropin, thyroxine and L-thyronine.

Lipids and lipid carriers also can be used to supplement cell culture media, depending on the type of cell and the fate of the differentiated cell. Such lipids and carriers can include, but are not limited to, cholesterol, linoleic acid conjugated to albumin, cyclodextrin, linoleic acid and oleic acid conjugated to albumin, unconjugated linoleic acid, linoleic-oleic-arachidonic acid conjugated to albumin and oleic acid unconjugated and conjugated to albumin, among others.

HSPCs can be cultured in low-serum or serum-free culture medium. Serum-free medium used to culture cells is described in, for example, U.S. Pat. No. 7,015,037. Many cells have been grown in serum-free or low-serum medium. For example, the medium can be supplemented with one or more growth factors. Commonly used growth factors include, but are not limited to, bone morphogenic protein, basic fibroblast growth factor, platelet-derived growth factor and epidermal growth factor, Stem cell factor, thrombopoietin, Flt3Ligand and 1′-3. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; all incorporated by reference herein for teaching growing cells in serum-free medium.

Cells in culture can be maintained either in suspension or attached to a solid support, such as extracellular matrix components. Stem cells often require additional factors that encourage their attachment to a solid support, such as type I and type II collagen, chondroitin sulfate, fibronectin, “superfibronectin” and fibronectin-like polymers, gelatin, poly-D and poly-L-lysine, thrombospondin and vitronectin. HSPCs can also be cultured in low attachment flasks such as but not limited to Corning Low attachment plates.

In one embodiment, hematopoietic stem and/or progenitor cells are treated ex vivo by contacting a population of hematopoetic cells with compositions comprising at least one SH2B3 inhibitor (i.e., SH2B3 iRNA or gene editing system) as described here into differentiate them into RBC, which are collected and then subsequently transplanted to an individual in need thereof. The effective concentration of the SH2B3 inhibitor can be determined by those of skill in the art, for example by performing serial dilutions and testing efficacy in an appropriate in vitro assay, or other suitable system. Example concentration ranges for the treatment of the hematopoietic stem and/or progenitor cells include, but are not limited to, about 1 nanomolar to about 10 millimolar; about 1 mM to about 5 mM; about 1 nM to about 500 nM; about 500 nM to about 1,000 nM; about 1 nM to about 1,000 nM; about 1 uM to about 1,000 uM; 1 uM to about 500 uM; about 1 uM to about 100 uM; about 1 uM to about 10 uM. In one embodiment, the range is about 5 uM to about 500 uM.

HSPCs can be treated for various times. Suitable times can be determined by those of skill in the art. For example, cells can be treated for minutes, e.g. 5 minutes, 10 minutes, 15 minutes, 30 minutes etc, or treated for hours e.g., 1 hour, 2 hours, 3 hours, 4 hours, up to 24 hours or even days. In one embodiment the cells are treated for 2 hours prior to changing to medium without a composition comprising at least one of the iRNAs described herein.

Once established in culture, cells treated as described herein to enhance HSPC populations, and/or untreated cells can be used fresh or frozen and stored as frozen stocks, using, for example, DMEM with 40% FCS and 10% DMSO. Other methods for preparing frozen stocks for cultured cells also are available to those skilled in the art.

In addition, RBCs obtained using the methods and compositions as disclosed herein can be expanded via the methods described herein, and can be subsequently cryopreserved using techniques known in the art for RBC cryopreservation. Accordingly, using cryopreservation, the RBCs can be maintained such that once it is determined that a subject is in need of a blood transfusion, the RBCs can be thawed and transplanted into the subject.

### Methods to Inhibit SH2B3

The inhibition of SH2B3 can result in a decrease in SH2B3 protein level, a decrease in SH2B3 mRNA level, a decrease in SH2B3 protein activity, or combinations thereof. The inhibition of SH2B3 can be done using a variety of methods known in the art including, but not limited to, genome editing, gene silencing, disruption of normal SH2B3 protein activity, and combinations thereof.

In some embodiments, SH2B3 can be inhibited in the stem cells and/or progenitor cells before the cells are expanded and/or enriched. In some embodiments, the stem cells and/or progenitor cells are expanded and/or enriched prior to SH2B3 inhibition.

In some embodiments, the inhibition of SH2B3 comprises contacting the population of stem cells and/or progenitor cells with a genome-editing agent for targeted excision of the SH2B3 gene from at least one stem cell. As used herein, the term “genome-editing agent” refers to a compound or a composition that can modify a nucleotide sequence in the genome of an organism. In some embodiments, the genome-editing agent can excise a specific nucleotide sequence from the target genome. In some embodiments, the genome-editing agent can disrupt the function of a specific nucleotide sequence, for example, by breaking one or more bonds in the sequence. Genome editing can be achieved through processes such as nuclease-mediated mutagenesis, chemical mutagenesis, radiation mutagenesis, or meganuclease-mediated mutagenesis.

In some embodiment, the genome-editing agent comprises a DNA-binding member and a nuclease, wherein the DNA-binding member localizes the nuclease to a target site which is then cut by the nuclease.

In some embodiments, the genome-editing agent is a CRISPR/Cas system. In some embodiments, the CRISPR/Cas system is CRISPR/Cas9, which is disclosed in U.S. Pat. No. 8,697,359 and US Application 2015/0291966, which is corporated herein in its entirety by reference. In alternative embodiments, the CRISPR/Cas system is CRISPR/Cpf1, as disclosed in Zetsche et al., 2015; Cell 163(3); 759-777 “Cpf1 Is a Single RNA-Guided Endonuclease of a Class 2 CRISPR-Cas System”, which is incorporated herein in its entirety by reference. The CRISPR/Cas is an engineered nuclease system based on a bacterial system that can be used for genome engineering. It is based on part of the adaptive immune response of many bacteria and archea. When a virus or plasmid invades a bacterium, segments of the invader's DNA are converted into CRISPR RNAs (crRNA) by the ‘immune’ response. This crRNA then associates, through a region of partial complementarity, with another type of RNA called tracrRNA to guide the Cas9 or Cpf1 nuclease to a region homologous to the crRNA in the target DNA called a “protospacer”. Cas9 cleaves the DNA to generate blunt ends at the double-strand break (DSB) at sites specified by a 20-nucleotide guide sequence contained within the crRNA transcript. Cas9 requires both the crRNA and the tracrRNA for site specific DNA recognition and cleavage. This system has now been engineered such that the crRNA and tracrRNA can be combined into one molecule (the “single guide RNA”), and the crRNA equivalent portion of the single guide RNA can be engineered to guide the Cas9 nuclease to target any desired sequence (see Jinek et al (2012) Science 337, p. 816-821, Jinek et al, (2013), eLife 2:e00471, and David Segal, (2013) eLife 2:e00563). In alternative embodiments, the CRISPR/Cpf1 system is used, where Cpf1 requires only one RNA template in the gene-editing complex and cleaves the DNA resulting in a 5 nt staggered cut distal to the 5′ T-rich PAM, resulting in sticky ends (rather than blunt ends as when Cas9 is used). In some embodiments, a replacement gene can be used in the place of a SH2B3 gene, e.g., a marker gene or in some embodiments, an cell death gene which is operatively linked to an inducible promoter, thereby allowing specific inducable cell death of the modified (i.e., SH2B3 gene deleted) cells with a drug to turn on expression from the inducible promoter, should it be necessary to eliminate such modified cells after they are transplanted into a subject. Accordingly, the CRISPR/Cas (cas9 or cpf1) system can be engineered to create a double strand break (i.e., blunt ends (i.e., using cas9)) or sticky ends (i.e., using cpf1)) at a desired target in a genome, and repair of the double strand break can be influenced by the use of repair inhibitors to cause an increase in error prone repair.

There are at least three types of CRISPR/Cas systems which all incorporate RNAs and Cas proteins. Types I and III both have Cas endonucleases that process the pre-crRNAs, that, when fully processed into crRNAs, assemble a multi-Cas protein complex that is capable of cleaving nucleic acids that are complementary to the crRNA. The Type II CRISPR (exemplified by Cas9) is one of the most well characterized systems. The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand.

In some embodiments, Cas protein can be a “functional derivative” of a naturally occurring Cas protein. As used herein, a “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof.

As used herein, “Cas polypeptide” encompasses a full-length Cas polypeptide, an enzymatically active fragment of a Cas polypeptide, and enzymatically active derivatives of a Cas polypeptide or fragment thereof. Suitable derivatives of a Cas polypeptide or a fragment thereof include, but are not limited to, mutants, fusions, covalent modifications of Cas protein or a fragment thereof

Cas proteins and Cas polypeptides can be obtained from a cell or synthesized chemically or by a combination of these two procedures. The cell can be a cell that naturally produces Cas protein, or a cell that naturally produces Cas protein and is genetically engineered to produce the endogenous Cas protein at a higher expression level or to produce a Cas protein from an exogenously introduced nucleic acid, which encodes a Cas that is same or different from the endogenous Cas. The cell can be a cell that does not naturally produce Cas protein and is genetically engineered to produce a Cas protein.

The CRISPR/Cas system can also be used to inhibit gene expression. Lei et al. (2013) Cell 152(5):1173-1183) have shown that a catalytically dead Cas9 lacking endonuclease activity, when coexpressed with a guide RNA, generates a DNA recognition complex that can specifically interfere with transcriptional elongation, RNA polymerase binding, or transcription factor binding. This system, called CRISPR interference (CRISPRi), can efficiently repress expression of targeted genes.

Additionally, Cas proteins have been developed which comprise mutations in their cleavage domains to render them incapable of inducing a DSB, and instead introduce a nick into the target DNA. In particular, the Cas nuclease comprises two nuclease domains, the HNH and RuvC-like, for cleaving the sense and the antisense strands of the target DNA, respectively. The Cas nuclease can thus be engineered such that only one of the nuclease domains is functional, thus creating a Cas nickase.

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome editing, both functions of these RNAs must be present (see Cong et al, (2013) Sciencexpress 1/10.1126/science 1231143). In some embodiments, the tracrRNA and pre-crRNAs are supplied via separate expression constructs or as separate RNAs. In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA).

The Cpf1 system, is related to the CRISPR/Cas9 system, although the Cpf1 protein is very different from Cas9, but is present in some bacteria with CRISPR. Cpf1 and Cas9 work differently, in that Cas9 requires two RNA molecules to cut DNA; Cpf1 needs only one. The proteins also cut DNA at different places, offering researchers more options when selecting a site to edit. Cpf1 also cuts DNA in a different way. Cas9 cuts both strands in a DNA molecule at the same position, leaving behind ‘blunt’ ends. In contrast, Cpf1 leaves one strand longer than the other, creating a ‘sticky’ end, reducing chances of abnormal/random DNA being inserted at the cleavage site, and also allowing better control of DNA to be inserted at the Cpf1 cleavage site. Cuts left by Cas9 tend to be repaired by sticking the two ends back together, that can leave errors. In contrast, Cpf1 sticky end cleavage allows more accurate and frequent insertions.

In some embodiments, the genome-editing agent is a ZFN. A ZFN generally comprises a zinc finger DNA binding protein and a DNA-cleavage domain. As used herein, a “zinc finger DNA binding protein” or “zinc finger DNA binding domain” is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein (ZFP). Zinc finger binding domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data.

In some embodiments, the genome-editing agent is a TALEN. As used herein, the term “transcription activator-like effector nuclease” or “TAL effector nuclease” or “TALEN” refers to a class of artificial restriction endonucleases that are generated by fusing a TAL effector DNA binding domain to a DNA cleavage domain. In some embodiments, the TALEN is a monomeric TALEN that can cleave double stranded DNA without assistance from another TALEN. The term “TALEN” is also used to refer to one or both members of a pair of TALENs that are engineered to work together to cleave DNA at the same site. TALENs that work together can be referred to as a left-TALEN and a right-TALEN, which references the handedness of DNA.

In some embodiments, a combination of genome-editing agents can be used.

In some embodiments, a CRISPR/Cas, TALEN, or ZFN molecule (e.g. a peptide and/or peptide/nucleic acid complex) can be introduced into a cell, e.g. a cultured stem cell or progenitor cell, such that the presence of the CRISPR/Cas, TALEN, or ZFN molecule is transient and will not be detectable in the progeny that cell. In some embodiments, a nucleic acid encoding a CRISPR/Cas, TALEN, or ZFN molecule (e.g. a peptide and/or multiple nucleic acids encoding the parts of a peptide/nucleic acid complex) can be introduced into a cell, e.g. a cultured stem cell or progenitor cell, such that the nucleic acid is present in the cell transiently and the nucleic acid encoding the CRISPR/Cas, TALEN, or ZFN molecule as well as the CRISPR/Cas, TALEN, or ZFN molecule itself will not be detectable in the progeny of that cell. In some embodiments, a nucleic acid encoding a CRISPR/Cas, TALEN, or ZFN molecule (e.g. a peptide and/or multiple nucleic acids encoding the parts of a peptide/nucleic acid complex) can be introduced into a cell, e.g. a cultured stem cell or progenitor cell, such that the nucleic acid is maintained in the cell (e.g. incorporated into the genome) and the nucleic acid encoding the CRISPR/Cas, TALEN, or ZFN molecule and/or the CRISPR/Cas, TALEN, or ZFN molecule will be detectable in the progeny of that cell.

The genome-editing agents can be delivered to a target cell by any suitable means. In some embodiments, the genome-editing agent (e.g., CRISPR/Cas, TALEN, or ZFN) is a protein and can be delivered by any suitable means for delivering a protein into a cell such as electroporation, sonoporation, microinjection, liposomal delivery, and nanomaterial-based delivery.

The genome-editing agent can also be encoded by a nucleotide sequence. In some embodiments, the genome-editing agent can be delivered using a vector known to those of ordinary skill in the art. Viral vector systems which can be utilized in the present invention include, but are not limited to, (a) adenovirus vectors; (b) retrovirus vectors; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g. canary pox or fowl pox; (j) a helper-dependent or gutless adenovirus; (k) a lentiviral vector; (1)adenovirus vectors; and (m) herpesvirus vectors. See, also, U.S. Pat. Nos. 6,534,261; 6,607,882; 6,824,978; 6,933,113; 6,979,539; 7,013,219; and 7,163,824, each of which are incorporated by reference herein in their entireties. Replication-defective viruses can also be advantageous.

In some embodiments, a plasmid expression vector can be used. Plasmid expression vectors include, but are not limited to, pcDNA3.1, pET vectors (Novagen®), pGEX vectors (GE Life Sciences), and pMAL vectors (New England labs. Inc.) for protein expression in E. coli host cell such as BL21, BL21(DE3) and AD494(DE3)pLysS, Rosetta (DE3), and Origami(DE3) ((Novagen®); the strong CMV promoter-based pcDNA3.1 (Invitrogen™ Inc.) and pCIneo vectors (Promega) for expression in mammalian cell lines such as CHO, COS, HEK-293, Jurkat, and MCF-7; replication incompetent adenoviral vector vectors pAdeno X, pAd5F35, pLP-Adeno-X-CMV (Clontech®), pAd/CMV/V5-DEST, pAd-DEST vector (Invitrogen™ Inc.) for adenovirus-mediated gene transfer and expression in mammalian cells; pLNCX2, pLXSN, and pLAPSN retrovirus vectors for use with the Retro-X™ system from Clontech for retroviral-mediated gene transfer and expression in mammalian cells; pLenti4/V5-DEST™, pLenti6/V5-DEST™, and pLenti6.2/V5-GW/lacZ (INVITROGEN™ Inc.) for lentivirus-mediated gene transfer and expression in mammalian cells; adenovirus-associated virus expression vectors such as pAAV-MCS and pAAV-IRES-hrGFP for adeno-associated virus-mediated gene transfer and expression in mammalian cells.

The vector may or may not be incorporated into the cell genome. The constructs may include viral sequences for transfection, if desired. Alternatively, the construct may be incorporated into vectors capable of episomal replication, e.g., EPV and EBV vectors.

When one or more ZFPs, TALENs, CRISPR/Cas molecules are introduced into the cell, the ZFPs, TALENs, CRISPR/Cas molecules can be carried on the same vector or on different vectors. When multiple vectors are used, each vector can comprise a sequence encoding one or multiple ZFPs, TALENs, CRISPR/Cas molecules.

Non-viral based delivery methods can also be used to introduce nucleic acids encoding engineered ZFPs, CRISPR/Cas molecules, and/or TALENs into cells (e.g., stem cells and/or progenitor cells). Methods of non-viral delivery of nucleic acids include electroporation, sonoporation, lipofection, microinjection, biolistics, virosomes, liposomes, immunoliposomes, polycation or lipid-nucleic acid conjugates, naked DNA, mRNA, artificial virions, and agent-enhanced uptake of DNA.

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424, WO 91/16024.

More details about genome-editing techniques can be found, for example, in “Targeted Genome Editing Using Site-Specific Nucleases: ZFNs, TALENs, and the CRISPR/Cas9 System” by Takashi Yamamoto (Springer, 2015), the contents of which are incorporated herein by reference for the teaching on genome editing.

### SH2B3 Antagonists

In some embodiments, the inhibition of SH2B3 comprises contacting the population of stem cells and/or progenitor cells with an antagonist of SH2B3. As used herein, the term “antagonist of SH2B3” refers to any agent that decreases the level and/or activity of SH2B3. The term “antagonist of SH2B3” refers to an agent which decreases the expression and/or activity SH2B3 by at least 10%, e.g. by at least 10%, at least 20%, at least 30%, at least 40%, at least 50%, at least 60%, at least 70%, at least 80%, or at least 90%. Examples of antagonists of SH2B3 include, but are not limited to, an inorganic molecule, an organic molecule, a nucleic acid, a nucleic acid analog or derivative, a peptide, a peptidomimetic, a protein, an antibody or an antigen-binding fragment thereof, and combinations thereof.

In some embodiments, the antagonist of SH2B3 is a nucleic acid or a nucleic acid analog or derivative thereof, also referred to as a nucleic acid agent herein. As will be appreciated by those skilled in the art, the depiction of a single strand also defines the sequence of the complementary strand. Thus, a nucleic acid also encompasses the complementary strand of a depicted single strand.

Without limitation, the nucleic acid agent can be single-stranded or double-stranded. A single-stranded nucleic acid agent can have double-stranded regions, e.g., where there is internal self-complementarity, and a double-stranded nucleic acid agent can have single-stranded regions. The nucleic acid can be of any desired length. In particular embodiments, nucleic acid can range from about 10 to 100 nucleotides in length. In various related embodiments, nucleic acid agents, single-stranded, double-stranded, and triple-stranded, can range in length from about 10 to about 50 nucleotides, from about 20 to about 50 nucleotides, from about 15 to about 30 nucleotides, from about 20 to about 30 nucleotides in length. In some embodiments, a nucleic acid agent is from about 9 to about 39 nucleotides in length. In some other embodiments, a nucleic acid agent is at least 30 nucleotides in length.

The nucleic acid agent can comprise modified nucleosides as known in the art. Modifications can alter, for example, the stability, solubility, or interaction of the nucleic acid agent with cellular or extracellular components that modify activity. In certain instances, it can be desirable to modify one or both strands of a double-stranded nucleic acid agent. In some cases, the two strands will include different modifications. In other instances, multiple different modifications can be included on each of the strands. The various modifications on a given strand can differ from each other, and can also differ from the various modifications on other strands. For example, one strand can have a modification, and a different strand can have a different modification. In other cases, one strand can have two or more different modifications, and the another strand can include a modification that differs from the at least two modifications on the first strand.

In some embodiments, the antagonist of SH2B3 is a single-stranded and double-stranded nucleic acid agent that is effective in inducing RNA interference, referred to as siRNA, RNAi agent, or iRNA agent herein. iRNA agents suitable for inducing RNA interference in SH2B3 are disclosed, for example, in WO2013/019857, the contents of which are incorporated herein by reference in their entirety.

RNAi Inhibitors of SH2B3

In one embodiment, the iRNA agent includes double-stranded ribonucleic acid (dsRNA) molecules for inhibiting the expression of a gene encoding SH2B3 in a cell, e.g., a cell in a population of human stem cells and/or progenitor cells, where the dsRNA includes an antisense strand having a region of complementarity which is complementary to at least a part of an mRNA formed in the expression of a gene encoding SH2B3, and where the region of complementarity is 30 nucleotides or less in length, generally 19-24 nucleotides in length, and where the dsRNA, upon contact with or introduction to a cell expressing the gene SH2B3, inhibits the expression of the gene by at least 10% as assayed by, for example, a PCR or branched DNA (bDNA)-based method, or by a protein-based method, such as by immunoassay or Western blot. Expression of SH2B3 in cell culture can be assayed by measuring SH2B3 mRNA levels, such as by bDNA or TaqMan assay, or by measuring protein levels, such as by immunofluorescence analysis, using, for example, Western Blotting or flow cytometric techniques.

In some embodiments, the iRNA agent is an antisense oligonucleotide. One of skill in the art is well aware that single-stranded oligonucleotides can hybridize to a complementary target sequence and prevent access of the translation machinery to the target RNA transcript, thereby preventing protein synthesis. The single-stranded oligonucleotide can also hybridize to a complementary RNA and the RNA target can be subsequently cleaved by an enzyme such as RNase H and thus preventing translation of target RNA. Alternatively, or in addition, the single-stranded oligonucleotide can modulate the expression of a target sequence via RISC mediated cleavage of the target sequence, i.e., the single-stranded oligonucleotide acts as a single-stranded RNAi agent. A “single-stranded RNAi agent” as used herein, is an RNAi agent which is made up of a single molecule. A single-stranded RNAi agent can include a duplexed region, formed by intra-strand pairing, e.g., it can be, or include, a hairpin or pan-handle structure.

In some embodiments, the iRNA agent is a small hairpin RNA or short hairpin RNA (shRNA), a sequence of RNA that makes a tight hairpin turn that can be used to silence target gene expression via RNA interference (RNAi).

Without wishing to be bound by theory, SH2B3 is also known by aliases SH2B adaptor protein 3 or Lnk; NCBI Gene ID: 10019) is a member of intracellular adaptor protein family. The mRNA of SH2B3 for homo sapiens is known to have at least two isoforms (NCBI Accession No: NM_005475.5 (transcript variant 1) and NM_001291424 (transcript variant 2). SH2B3 encoded by transcript variant 1 has an amino acid sequence of NP_005466.1 (SEQ ID NO: 51) which is as follows:

(SEQ ID NO: 51)

MNGPALQPSSPSSAPSASPAAAPRGWSEFCELHAVAAARELARQYWLFAR
EHPQHAPLRAELVSLQFTDLFQRYFCREVRDGRAPGRDYRDTGRGPPAKA
EASPEPGPGPAAPGLPKARSSEELAPPRPPGPCSFQHFRRSLRHIFRRRS
AGELPAAHTAAAPGTPGEAAETPARPGLAKKFLPWSLAREPPPEALKEAV
LRYSLADEASMDSGARWQRGRLALRRAPGPDGPDRVLELFDPPKSSRPKL
QAACSSIQEVRWCTRLEMPDNLYTFVLKVKDRTDIIFEVGDEQQLNSWMA
ELSECTGRGLESTEAEMHIPSALEPSTSSSPRGSTDSLNQGASPGGLLDP
ACQKTDHFLSCYPWFHGPISRVKAAQLVQLQGPDAHGVFLVRQSETRRGE
YVLTFNFQGIAKHLRLSLTERGQCRVQHLHFPSVVDMLHHFQRSPIPLEC
GAACDVRLSSYVVVVSQPPGSCNTVLFPFSLPHWDSESLPHWGSELGLPH
LSSSGCPRGLSPEGLPGRSSPPEQIFHLVPSPEELANSLQHLEHEPVNRA
RDSDYEMDSSSRSHLRAIDNQYTPL.

Inhibition of the SH2B3 gene can be by gene silencing RNAi molecules according to methods commonly known by a skilled artisan. For example, a gene silencing siRNA oligonucleotide duplexes targeted specifically to human SH2B3 (GenBank No: NM_005475.5 or NM_001291424) can readily be used to knockdown SH2B3 expression. SH2B3 mRNA can be successfully targeted using siRNAs; and other siRNA molecules may be readily prepared by those of skill in the art based on the known sequence of the target mRNA. To avoid doubt, the sequence of a human SH2B3 is provided at, for example, GenBank Accession Nos. NM_005475.5 (SEQ ID NO: 52). Accordingly, in avoidance of any doubt, one of ordinary skill in the art can design nucleic acid inhibitors, such as RNAi (RNA silencing) agents to the nucleic acid sequence of NM_001005735.1 (SEQ ID NO: 52) which is as follows:

| (SEQ ID NO: 52) | |
|---|---|
| 1 | cccgggccac cgcctccgcc cggctgcccg cccggactgt cgcggcccgc ggtggcgacg |
| 61 | gcggccgctg caaagtttcc ccggcggcgg cggcccgggg gcgcatcctc ccgcaactgt |
| 121 | caagcgctgg cggcggaaat gatgaggcgc tggccatttt ccgagcccgg gtttcctgcc |
| 181 | tgagccccgc tcgagcgagc cgcgagcgag gagccggcgg gcgggagagg acgcgcccag |
| 241 | ggcgggggcc cgcccgcccc ctcgggattt cgagggcccg ggggcgcgcg acgccatggg |
| 301 | ccggccgggc ccagagctcc tgtctctcag cccggccgca ccacctgggt ctccgccatg |
| 361 | aacgggcctg ccctgcagcc ctcctcgccc tcttccgcgc cctcagcctc cccggcggcg |
| 421 | gccccgcggg gctggagcga gttctgtgag ttgcacgccg tagcggcggc ccgggagctg |
| 481 | gcccgccagt actggctgtt cgcccgggag catccgcagc acgcgccgct gcgcgccgag |
| 541 | ctggtgtcgc tgcagttcac cgacctcttc cagcgctact tctgccgcga ggtgcgcgac |
| 601 | ggacgggcgc cgggccgcga ctaccgggac acaggccgtg ggcccccagc caaggccgag |
| 661 | gcgtccccgg agccaggccc cggccccgcc gcccctggcc tgcccaaggc ccgcagctct |
| 721 | gaggagctgg ccccgccgcg gccgcccggg ccctgctcct tccagcactt tcgccgcagc |
| 781 | ctccgccaca tcttccgccg ccgctcggcc ggggagctgc cagcggccca caccgctgcc |
| 841 | gcccccggga cccccggaga ggctgctgag acccccgccc ggcctggcct ggccaagaag |
| 901 | ttcctgccct ggagcctggc ccgggagccg ccacccgagg cgctgaagga ggcggtgctg |
| 961 | cgctacagcc tggccgacga ggcctccatg gacagcgggg cacgctggca gcgcgggagg |
| 1021 | ctggcgctgc gccgggcccc gggccccgat ggccccgacc gcgtgctgga gctcttcgac |
| 1081 | ccacccaaga gttcaaggcc caagctacaa gcagcttgct ccagcatcca ggaggtccgg |
| 1141 | tggtgcacac ggcttgagat gcctgacaac ctttacacct ttgtgctgaa ggtgaaggac |
| 1201 | cggacagaca tcatctttga ggtgggagac gagcagcagc tgaattcatg gatggctgag |
| 1261 | ctctcggagt gcacaggccg agggctggag agcacagaag cagagatgca tattccctca |
| 1321 | gccctagagc ctagcacgtc cagctcccca aggggcagca cagattccct taaccaaggt |
| 1381 | gcttctcctg gggggctgct ggacccggcc tgccagaaga cggaccattt cctgtcctgc |
| 1441 | tacccctggt tccacggccc catctccaga gtgaaagcag ctcagctggt tcagctgcag |
| 1501 | ggccctgatg ctcatggagt gttcctggtg cggcagagcg agacgcggcg tggggaatac |
| 1561 | gtgctcactt tcaactttca ggggatagcc aagcacctgc gcctgtcgct gacagagcgg |
| 1621 | ggccagtgcc gtgtgcagca cctccacttt ccctcggtcg tggacatgct ccaccacttc |
| 1681 | cagcgctcgc ccatcccact cgagtgcggc gccgcctgtg atgtccggct ctccagctac |
| 1741 | gtggtagtcg tctcccaacc accaggttcc tgcaacacgg tcctcttccc tttctccctt |
| 1801 | cctcactggg attcagagtc ccttcctcac tggggttcag agttgggcct tccccacctt |
| 1861 | agttcttctg gctgtccccg ggggctcagc ccagagggtc tcccagggcg atcctcaccc |
| 1921 | cccgagcaga tcttccacct ggtgccttcg cccgaagaac tggccaacag cctgcagcac |
| 1981 | ctggagcatg agcctgtgaa tcgagcccgg gactcggact acgaaatgga ctcatcctcc |
| 2041 | cggagccacc tgcgggccat agacaatcag tacacacctc tctgaccagt gaggaattcc |
| 2101 | aggcctcaac agctgccctt gaggagcaca ggcagaagtg tgaacttgtg aatgtaattg |
| 2161 | atctttcctt ccttccagag aaagatttaa gggacactgt taactgctcg tgccagtttg |
| 2221 | gaagtgaccc ttctattagg cctgttgaag ggccctcctg taggtttcat ctatccacct |
| 2281 | ggctttctcc ttattgttta cagatgtagt tcttgttaga ggatgccgct agctcctgcc |
| 2341 | cggggtccct atgcccagtc cccgttactc ttagagaaag gagttggggt gagggccaga |
| 2401 | gctggcagtg gaaacttgtt ctctttttca ctgacactgt cacagcggat gacagacttt |
| 2461 | ctacggggag gaggggggga tcatcaggaa gcccagaaca ctaacaagcg gttctcccat |
| 2521 | ctaccgtcag tccacatggc aggtctgctg tgtccacacc acagatgacc acatctaatc |
| 2581 | ctgcttctac tctcagcttt aggacaaaag ctctgtcaga ggcacaagct gaaggtcaaa |
| 2641 | aatgatttaa aacattttac ctcagactaa tttctttaaa ggattcaggt tcaaaactta |
| 2701 | accactgctt atttcagtgc actgtttcaa ctaacaccca tgctattttt gtagtcagaa |
| 2761 | acagctatgc aaaccctacc taatttacag tctgagccag catgctggct tgtctactgc |
| 2821 | atcctcggga cagtcacctg ccactgagtg gccactgtcc ttcctaaatg tcaagaagtg |
| 2881 | aagtatgtca ccctttcagg gaaattcagg caattactga aataggaggg tggcaagaac |
| 2941 | agttctatcc tggtgcctta cgaataaaaa actggattct ggtttacagc agctttacag |
| 3001 | tgatagttaa attaactggg gctaggggaa gagcaagcaa aaagggaaga aggactccta |
| 3061 | ggccctttct agtaaatcct tcagcaacaa ggctggcttg gtgccctcca agcatctaat |
| 3121 | ggcttattaa attatcccac aagtgggttt taggctcctt ttttgagcca aaatggaagc |
| 3181 | tgggaatctg gtgccataac taatgagaaa ctcctttaat agcccacaat cagtgttctg |
| 3241 | ttctagctgg ctactgcttc actggattga gaatctatct atctccttgc acacatgggc |
| 3301 | acacacaatc tccaccatcc agggaggtcc tgaagtcaaa tctctatcta tacaagtgat |
| 3361 | acaattcata gggggctggc tcctcccaga acctgtctgg aggctcagaa acgggggcag |
| 3421 | tgacagtgga gtcagctgct cttgggtgcc agcagagcca ttcagtacaa cccccaggct |
| 3481 | cacagcagtg gcttctagga aactgggagt ttagatcagc tttacagata catcgatcag |
| 3541 | aggctaaaat gaaacctcag cctaaaactc ataggactga ctgcctggga ggagggttag |
| 3601 | gtctgcttct tccacttata cttagtctct gtgctccaag aggtcaaatt tttgcttcta |
| 3661 | gaatttcctt ggggtctttc agagggtggg ggaacaaacc cctatgcact tttctttttt |
| 3721 | ttttttttga gatggagttt ctcttgtcaa ccgggctgga gtgcagtggt gcaatcttgg |
| 3781 | ctcactgcaa cctccacctt cctggttcaa gcgattctgc ctcgacctct caagtagctg |
| 3841 | ggattacaag caccagccac catgcctggc taattttgta tttttagtag agacagggtt |
| 3901 | tcaccatgtt ggccaggctg gtctcgaatg tctgacctca ggtgatccac ccgccttggc |
| 3961 | ctcccaaagt gctgggatta caggcgcgag ccaccgcgcc cagcctacac cacttttagt |
| 4021 | accaacactc ttgggtgatt tcatggaccc taaagcagac ctgacactga tccagatttg |
| 4081 | cagtccattt ttaaggacac ctgtctttat ttcctcaaag tcaagcagct ttctctggaa |
| 4141 | aatgaatgct aattagtgtg aaccaaaaga gtaagtaaga gtctgaagtt tttttaaagg |
| 4201 | agaaagctta ttatggaaag tcactggtcc tcccctccgc acaggaaagg tacccagtag |
| 4261 | ataatgaacc aaattaagtt ccctccctcc agccagaagt taaacatctg ggatatgacg |
| 4321 | tcttcatgcc aggggcactc atttcttagc agcctctcta catacatctc tcaggtggtg |
| 4381 | ccaagaggca caccaggtag agcaaactta gcagctctga ctaacaggct gcaaagtgca |
| 4441 | agttcagatt ctgtggcaga gatttggaag gcacccacct ccagactgct tcccgtccaa |
| 4501 | gttaccagga cagctcaaaa acatgctgac agaaaactcc catggctcta ggaagaagtg |
| 4561 | acactaagcc aacacctttc tttatgtggg agcagaatca gctgatgaag gggtgggcag |
| 4621 | cagtgtgggg caggcacccc actggctgca gctagcccac cataggcaca gcacatccca |
| 4681 | ccactctcct tccagtcctg accaggcccc agccggcaac ttctaccgag agccatggct |
| 4741 | caacaccaaa ctggacagta gacatcatga tccctccagt tagctctaat tacagacccc |
| 4801 | accagtacag cttgacagct cccggcacca tcccttcctt catctgactt attgaacttt |
| 4861 | tacaaactaa cagtcaccag caccaaagaa ttaagtcaac taacctgcct tgaattttag |
| 4921 | accagcaatc catatggctt tatctggtat aaatcttctg cctttgatca tttctggacc |
| 4981 | gtaggaaaaa ggaatagcaa tcattaaaat cttgggccag agaacactat ttttacataa |
| 5041 | cagtttctta acctaaagtc aaggccttgg actcttccct gagggttgcc tgagattcct |
| 5101 | tcatgctttc tattcaggac taagtccctt actgcaaatg tgttagctct aacatctccc |
| 5161 | acaagctaga ggaacttgcg agtatattaa caaggacaca tctgacatcc tgtgtttggt |
| 5221 | tagaatatac agcacattgt gataacataa agtggattca tcttgtatca ttataggcag |
| 5281 | aaggtatttg gcaaattttt atgtattgtt ttatgtactg tacaagtaac ttattcttga |
| 5341 | ataatgcaaa ttttgctata atgtacaaat tgctatatgt gaattaaaaa gttttcagaa |
| 5401 | tcttgaaaaa aaaaaaaaaa aaaaa. |

In some embodiments, the shRNA for targeting SH2B3 has a nucleotide sequence of CCGGCCTGACAACCTTTACACCTTTCTCGAGAAAGGTGTAAAGGTTGTCAGGTTTTTG (SEQ ID NO: 1) or a fragment of at least 10, at least 15, at least 20, or at least 25 contiguous nucleotides thereof.

In some embodiments, the shRNA for targeting SH2B3 has a nucleotide sequence of CCGGGCCTGACAACCTTTACACCTTCTCGAGAAGGTGTAAAGGTTGTCAGGCTTTTTG (SEQ ID NO: 2) or a fragment of at least 10, at least 15, at least 20, or at least 25 contiguous nucleotides thereof.

In some embodiments, polynucleotides of SEQ ID NO: 1 and SEQ ID NO: 2 or a fragment of at least 10, at least 15, at least 20, or at least 25 contiguous nucleotides thereof can be used in combination for targeting SH2B3.

In some embodiments, an antagonist of SH2B3 is a miRNA (also referred to as “miR”). miRs that have been shown to target SH2B3 include, but are not limited to; miR-153, miR-9, miR-181, miR-101, miR-384/384-3p, miR-124/506, miR-320/320abcd, miR-138, miR-421, miR-320/320abcd, miR-30a/30a-5p/30b/30b-5p/30cde/384-5p, miR-134, miR-488, miR-300, miR-148/152, miR-202/202-3p, miR-30a/30a-5p/30b/30b-5p/30cde/384-5p, miR-384/384-3p, miR-138, miR-33/33ab, miR-125/351, miR-326/330/330-5p, miR-326/330/330-5p, miR-23ab, miR-144, miR-448, and let-7/98.

In general, any method of delivering a nucleic acid molecule can be adapted for use with the nucleic acid agents described herein.

Methods of delivering RNA interference agents, e.g., an siRNA, or vectors containing an RNA interference agent, to the target cells, e.g., stem cells and/or progenitor cells, for uptake include injection of a composition containing the RNA interference agent, e.g., an siRNA, or directly contacting the cell with a composition comprising an RNA interference agent, e.g., an siRNA. In another embodiment, RNA interference agent, e.g., an siRNA may be injected directly into any blood vessel, such as vein, artery, venule or arteriole, via, e.g., hydrodynamic injection or catheterization. Administration may be by a single injection or by two or more injections. The RNA interference agent is delivered in a pharmaceutically acceptable carrier. One or more RNA interference agents may be used simultaneously. In one embodiment, specific cells are targeted with RNA interference, limiting potential side effects. The method can use, for example, a complex or a fusion molecule comprising a cell targeting moiety and an RNA interference binding moiety that is used to deliver RNA interference effectively into cells. For example, an antibody-protamine fusion protein when mixed with siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein. A viral-mediated delivery mechanism can also be employed to deliver siRNAs to cells in vitro and in vivo as described in Xia, H. et al. (2002) Nat Biotechnol 20(10):1006). Plasmid- or viral-mediated delivery mechanisms of shRNA may also be employed to deliver shRNAs to cells in vitro and in vivo as described in Rubinson, D. A., et al. ((2003) Nat. Genet. 33:401-406) and Stewart, S. A., et al. ((2003) RNA 9:493-501). The RNA interference agents, e.g., the siRNAs or shRNAs, can be introduced along with components that perform one or more of the following activities: enhance uptake of the RNA interfering agents, e.g., siRNA, by the cell, inhibit annealing of single strands, stabilize single strands, or otherwise facilitate delivery to the target cell and increase inhibition of the target gene, e.g., SH2B3. The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene.

### Oligonucleotide Modifications

In some embodiments, RNAi agents that inhibit SH2B3 for use in the aspects of the invention as disclosed herein can include oligonucleotide modifications. Unmodified oligonucleotides can be less than optimal in some applications, e.g., unmodified oligonucleotides can be prone to degradation by e.g., cellular nucleases. However, chemical modifications to one or more of the subunits of oligonucleotide can confer improved properties, e.g., can render oligonucleotides more stable to nucleases. Typical oligonucleotide modifications can include one or more of: (i) alteration, e.g., replacement, of one or both of the non-linking phosphate oxygens and/or of one or more of the linking phosphate oxygens in the phosphodiester intersugar linkage; (ii) alteration, e.g., replacement, of a constituent of the ribose sugar, e.g., of the 2′ hydroxyl on the ribose sugar; (iii) wholesale replacement of the phosphate moiety with “dephospho” linkers; (iv) modification or replacement of a naturally occurring base with a non-natural base; (v) replacement or modification of the ribose-phosphate backbone, e.g. peptide nucleic acid (PNA); (vi) modification of the 3′ end or 5′ end of the oligonucleotide, e.g., removal, modification or replacement of a terminal phosphate group or conjugation of a moiety, e.g., conjugation of a ligand, to either the 3′ or 5′ end of oligonucleotide; and (vii) modification of the sugar, e.g., six membered rings.

The terms replacement, modification, alteration, and the like, as used in this context, do not imply any process limitation, e.g., modification does not mean that one must start with a reference or naturally occurring ribonucleic acid and modify it to produce a modified ribonucleic acid bur rather modified simply indicates a difference from a naturally occurring molecule. As described below, modifications, e.g., those described herein, can be provided as asymmetrical modifications.

A modification described herein can be the sole modification, or the sole type of modification included on multiple nucleotides, or a modification can be combined with one or more other modifications described herein. The modifications described herein can also be combined onto an oligonucleotide, e.g. different nucleotides of an oligonucleotide have different modifications described herein.

Described herein are iRNA agents that inhibit the expression of SH2B3. In one embodiment, the iRNA agent includes double-stranded ribonucleic acid (dsRNA) molecules for inhibiting the expression of SH2B3 in a cell ex vivo, e.g., in HSPCs ex vivo obtained from blood or UCB, where the dsRNA includes an antisense strand having a region of complementarity which is complementary to at least a part of an mRNA formed in the expression of SH2B3, and where the region of complementarity is 30 nucleotides or less in length, generally 19-24 nucleotides in length, and where the dsRNA, upon contact with or introduction to a cell expressing the gene encoding SH2B3, inhibits the expression of the gene by at least 10% as assayed by, for example, a PCR or branched DNA (bDNA)-based method, or by a protein-based method, such as by immunoassay or Western blot. Expression of SH2B3 in cell culture, such as HSPCs, can be assayed by measuring mRNA levels of SH2B3, such as by bDNA or TaqMan assay, or by measuring protein levels, such as by immunofluorescence analysis, using, for example, Western Blotting or flowcytometric techniques.

A dsRNA includes two RNA strands that are complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of SH2B3 mRNA, e.g, SEQ ID NO: 52 as disclosed herein. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

One of skill in the art will also recognize that the duplex region is a primary functional portion of a dsRNA, e.g., a duplex region of 9 to 36, e.g., 15-30 base pairs. Thus, in one embodiment, to the extent that it becomes processed to a functional duplex of e.g., 15-30 base pairs that targets a desired RNA for cleavage, an RNA molecule or complex of RNA molecules having a duplex region greater than 30 base pairs is a dsRNA. Thus, an ordinarily skilled artisan will recognize that in one embodiment, then, an miRNA is a dsRNA. In another embodiment, a dsRNA is not a naturally occurring miRNA. In another embodiment, an iRNA agent useful to target expression of SH2B3 is not generated in the target cell by cleavage of a larger dsRNA.

A dsRNA as described herein can further include one or more single-stranded nucleotide overhangs. The dsRNA can be synthesized by standard methods known in the art as further discussed below, e.g., by use of an automated DNA synthesizer, such as are commercially available from, for example, Biosearch, Applied Biosystems, Inc. In one embodiment, a gene encoding SH2B3 is a human gene. In another embodiment the gene encoding SH2B3 is a mouse or rat gene.

In one aspect, a dsRNA will include at least two nucleotide sequences, a sense and an anti-sense sequence, wherein the sense strand is SEQ ID NO: 52. In this aspect, one of the two sequences is complementary to the other of the two sequences, with one of the sequences being substantially complementary to a sequence of the SH2B3 mRNA. As described elsewhere herein and as known in the art, the complementary sequences of a dsRNA can also be contained as self-complementary regions of a single nucleic acid molecule, as opposed to being on separate oligonucleotides.

The skilled person is well aware that dsRNAs having a duplex structure of between 20 and 23, but specifically 21, base pairs have been hailed as particularly effective in inducing RNA interference (Elbashir et al., EMBO 2001, 20:6877-6888). However, others have found that shorter or longer RNA duplex structures can be effective as well. In the embodiments described above, by virtue of the nature of the oligonucleotide sequences identified by sequence identifiers provided in Tables 2-7, dsRNAs described herein can include at least one strand of a length of minimally 21 nt. It can be reasonably expected that shorter duplexes having one of the sequences of Tables 2-7 minus only a few nucleotides on one or both ends can be similarly effective as compared to the dsRNAs described above. Hence, dsRNAs having a partial sequence of at least 15, 16, 17, 18, 19, 20, or more contiguous nucleotides from one of the sequences of SEQ ID NO: 1 or 2, and differing in their ability to inhibit the expression of a gene encoding SH2B3 by not more than 5, 10, 15, 20, 25, or 30% inhibition from a dsRNA comprising the full sequence, are contemplated according to the technology described herein.

While a target sequence is generally 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an iRNA agent, mediate the best inhibition of target gene expression. Thus, while the sequences identified, for example, by sequence identifiers in Tables 2-7 represent effective target sequences, it is contemplated that further optimization of inhibition efficiency can be achieved by progressively “walking the window” one nucleotide upstream or downstream of the given sequences to identify sequences with equal or better inhibition characteristics.

Further, it is contemplated that for any sequence identified by a sequence identifier NO: 1 or 2, can be further optimization could be achieved by systematically either adding or removing nucleotides to generate longer or shorter sequences and testing those and sequences generated by walking a window of the longer or shorter size up or down the target RNA from that point. Again, coupling this approach to generating new candidate targets with testing for effectiveness of iRNAs based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

An iRNA as described herein can contain one or more mismatches to the target sequence. In one embodiment, an iRNA as described herein contains no more than 3 mismatches. If the antisense strand of the iRNA contains mismatches to a target sequence, it is preferable that the area of mismatch not be located in the center of the region of complementarity. If the antisense strand of the iRNA contains mismatches to the target sequence, it is preferable that the mismatch be restricted to be within the last 5 nucleotides from either the 5′ or 3′ end of the region of complementarity. For example, for a 23 nucleotide iRNA agent RNA strand which is complementary to a region of a gene encoding SH2B3, the RNA strand generally does not contain any mismatch within the central 13 nucleotides. The methods described herein or methods known in the art can be used to determine whether an iRNA containing a mismatch to a target sequence is effective in inhibiting the expression of SH2B3. Consideration of the efficacy of iRNAs with mismatches in inhibiting expression of SH2B3 is important, especially if the particular region of complementarity to the SH2B3 gene is known to have polymorphic sequence variation within the population.

In one embodiment, at least one end of a dsRNA has a single-stranded nucleotide overhang of 1 to 4, generally 1 or 2 nucleotides. dsRNAs having at least one nucleotide overhang have unexpectedly superior inhibitory properties relative to their blunt-ended counterparts. In yet another embodiment, the RNA of an iRNA, e.g., a dsRNA, is chemically modified to enhance stability or other beneficial characteristics. The nucleic acids featured in the technology described herein can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, each of which is herein incorporated by reference

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH.sub.2 component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other embodiments, suitable RNA mimetics suitable are contemplated for use in iRNAs, in which both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Modifications of phosphate group: The phosphate group in the intersugar linkage can be modified by replacing one of the oxygens with a different substituent. One result of this modification to RNA phosphate intersugar linkages can be increased resistance of the oligonucleotide to nucleolytic breakdown. Examples of modified phosphate groups include phosphorothioate, phosphoroselenates, borano phosphates, borano phosphate esters, hydrogen phosphonates, phosphoroamidates, alkyl or aryl phosphonates and phosphotriesters. In some embodiments, one of the non-bridging phosphate oxygen atoms in the intersugar linkage can be replaced by any of the following: S, Se, BR₃ (R is hydrogen, alkyl, aryl), C (i.e. an alkyl group, an aryl group, etc. . . . ), H, NR₂ (R is hydrogen, optionally substituted alkyl, aryl), or OR (R is optionally substituted alkyl or aryl). The phosphorous atom in an unmodified phosphate group is achiral. However, replacement of one of the non-bridging oxygens with one of the above atoms or groups of atoms renders the phosphorous atom chiral; in other words a phosphorous atom in a phosphate group modified in this way is a stereogenic center. The stereogenic phosphorous atom can possess either the “R” configuration (herein Rp) or the “S” configuration (herein Sp).

Phosphorodithioates have both non-bridging oxygens replaced by sulfur. The phosphorus center in the phosphorodithioates is achiral which precludes the formation of oligonucleotides diastereomers. Thus, while not wishing to be bound by theory, modifications to both non-bridging oxygens, which eliminate the chiral center, e.g. phosphorodithioate formation, can be desirable in that they cannot produce diastereomer mixtures. Thus, the non-bridging oxygens can be independently any one of O, S, Se, B, C, H, N, or OR (R is alkyl or aryl).

The phosphate linker can also be modified by replacement of bridging oxygen, (i.e. oxygen that links the phosphate to the nucleoside), with nitrogen (bridged phosphoroamidates), sulfur (bridged phosphorothioates) and carbon (bridged methylenephosphonates). The replacement can occur at the either one of the linking oxygens or at both linking oxygens. When the bridging oxygen is the 3′-oxygen of a nucleoside, replacement with carbon is preferred. When the bridging oxygen is the 5′-oxygen of a nucleoside, replacement with nitrogen is preferred.

Modified phosphate linkages where at least one of the oxygen linked to the phosphate has been replaced or the phosphate group has been replaced by a non-phosphorous group, are also referred to as “non-phosphodiester intersugar linkage” or “non-phosphodiester linker.”

Replacement of the phosphate group: The phosphate group can be replaced by non-phosphorus containing connectors, e.g. dephospho linkers. Dephospho linkers are also referred to as non-phosphodiester linkers herein. While not wishing to be bound by theory, it is believed that since the charged phosphodiester group is the reaction center in nucleolytic degradation, its replacement with neutral structural mimics should impart enhanced nuclease stability. Again, while not wishing to be bound by theory, it can be desirable, in some embodiment, to introduce alterations in which the charged phosphate group is replaced by a neutral moiety.

Examples of moieties which can replace the phosphate group include, but are not limited to, amides (for example amide-3 (3′-CH₂—C(═O)—N(H)-5′) and amide-4 (3′-CH₂—N(H—C(═O)-5′)), hydroxylamino, siloxane (dialkylsiloxxane), carboxamide, carbonate, carboxymethyl, carbamate, carboxylate ester, thioether, ethylene oxide linker, sulfide,sulfonate, sulfonamide, sulfonate ester, thioformacetal (3′-S—CH₂—O-5′), formacetal (3′-O—CH₂—O-5′), oxime, methyleneimino, methykenecarbonylamino, methylenemethylimino (MMI, 3′-CH₂—N(CH₃)—O5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′ and nonionic linkages containing mixed N, O, S and CH₂ component parts. See for example, Carbohydrate Modifications in Antisense Research; Y. S. Sanghvi and P. D. Cook Eds. ACS Symposium Series 580; Chapters 3 and 4, (pp. 40-65). Preferred embodiments include methylenemethylimino (MMI), methylenecarbonylamino, amides,carbamate and ethylene oxide linker.

One skilled in the art is well aware that in certain instances replacement of a non-bridging oxygen can lead to enhanced cleavage of the intersugar linkage by the neighboring 2′-OH, thus in many instances, a modification of a non-bridging oxygen can necessitate modification of 2′-OH, e.g., a modification that does not participate in cleavage of the neighboring intersugar linkage, e.g., arabinose sugar, 2′-O-alkyl, 2′-F, LNA and ENA.

Preferred non-phosphodiester intersugar linkages include phosphorothioates, phosphorothioates with an at least 1%, 5%, 10%, 20%, 30%, 40%, 50%, 60%, 70%, 80%, 90% 95% or more enantiomeric excess of Sp isomer, phosphorothioates with an at least 1%, 5%, 10%, 20%, 30%, 40%, 50%, 60%, 70%, 80%, 90% 95% or more enantiomeric excess of Rp isomer, phosphorodithioates, phsophotriesters, aminoalkylphosphotrioesters, alkyl-phosphonaters (e.g., methyl-phosphonate), selenophosphates, phosphoramidates (e.g., N-alkylphosphoramidate), and boranophosphonates.

Replacement of ribophosphate backbone: Oligonucleotide-mimicking scaffolds can also be constructed wherein the phosphate linker and ribose sugar are replaced by nuclease resistant nucleoside or nucleotide surrogates. While not wishing to be bound by theory, it is believed that the absence of a repetitively charged backbone diminishes binding to proteins that recognize polyanions (e.g. nucleases). Again, while not wishing to be bound by theory, it can be desirable in some embodiment, to introduce alterations in which the bases are tethered by a neutral surrogate backbone. Examples include the morpholino, cyclobutyl, pyrrolidine, peptide nucleic acid (PNA), aminoethylglycyl PNA (aegPNA) and backnone-extended pyrrolidine PNA (bepPNA) nucleoside surrogates. In some embodiments, the oligonucleotide is a peptide nucleic acid, e.g., the ribophosphate backbone of the oligonucleotide is completely replaced by peptide nucleic acid (PNA).

Sugar modifications: An oligonucleotide can include modification of all or some of the sugar groups of the nucleic acid. For example, the 2′ position (H, DNA; or OH, RNA) can be modified with a number of different “oxy” or “deoxy” substituents. While not being bound by theory, enhanced stability is expected since the 2′-hydroxyl can no longer be deprotonated to form a 2′-alkoxide ion. The 2′-alkoxide can catalyze degradation by intramolecular nucleophilic attack on the linker phosphorus atom. Again, while not wishing to be bound by theory, it can be desirable to some embodiments to introduce alterations in which alkoxide formation at the 2′ position is not possible

Examples of “oxy”-2′ hydroxyl group modifications include alkoxy or aryloxy (OR, e.g., R═H, alkyl, cycloalkyl, aryl, aralkyl, heteroaryl or sugar); polyethyleneglycols (PEG), O(CH₂CH₂O)ₙCH₂CH₂OR, n=1-50; “locked” nucleic acids (LNA) in which the oxygen at the 2′ position is connected by (CH₂)ₙ, wherein n=1-4, to the 4′ carbon of the same ribose sugar, preferably n is 1 (LNA) or 2 (ENA); O-AMINE or O—(CH₂)ₙAMINE (n=1-10, AMINE=NH₂; alkylamino, dialkylamino, heterocyclyl, arylamino, diaryl amino, heteroaryl amino, diheteroaryl amino, ethylene diamine or polyamino); and O—CH₂CH₂(NCH₂CH₂NMe₂)₂.

Examples of “deoxy” modifications include halo (e.g., fluoro); amino (e.g. NH₂; alkylamino, dialkylamino, heterocyclyl, arylamino, diaryl amino, heteroaryl amino, diheteroaryl amino, or amino acid); NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino); —NHC(O)R (R=alkyl, cycloalkyl, aryl, aralkyl, heteroaryl or sugar); cyano; mercapto; alkyl-thio-alkyl; thioalkoxy; thioalkyl; alkyl; cycloalkyl; aryl; alkenyl and alkynyl, which can be optionally substituted with e.g., an amino functionality.

The sugar group can also contain one or more carbons that possess the opposite stereochemical configuration than that of the corresponding carbon in the ribose sugar. Thus, an oligonucleotide can include nucleotides containing e.g., arabinose, as the sugar. Similarly, a modification at the 2′ position can be present in the arabinose configuration The term “arabinose configuration” refers to the placement of a substituent on the C2′ of ribose in the same configuration as the 2′-OH is in the arabinose.

A nucleotide can have an alpha linkage at the 1′ position on the sugar, e.g., alpha-nucleosides. The monomer can also have the opposite configuration at the 4′-position, e.g., C5′ and H4′ or substituents replacing them are interchanged with each other. When the C5′ and H4′ or substituents replacing them are interchanged with each other, the sugar is said to be modified at the 4′ position.

Oligonucleotides can also include abasic sugars, i.e., monomers which lack a nucleobase at C-1′ or has other chemical groups in place of a nucleobase at C1′. See for example U.S. Pat. No. 5,998,203, contents of which are herein incorporated in their entirety. These abasic sugars can also be further containing modifications at one or more of the constituent sugar atoms. Oligonucleotides can also contain one or more sugars that are the L isomer, e.g. L-nucleosides. Modification to the sugar group can also include replacement of the 4′-O with a sulfur, optionally substituted nitrogen or CH₂ group. In some embodiments, linkage between C1′ and nucleobase is in the a configuration.

Oligonucleotide modifications can also include acyclic nucleotides, wherein a C—C bonds between ribose carbons (e.g., C1′-C2′, C2′-C3′, C3′-C4′, C4′-O4′, C1′-O4′) is absent and/or at least one of ribose carbons or oxygen (e.g., C1′, C2′, C3′, C4′ or O4′) are independently or in combination absent from the nucleotide. In some embodiments, acyclic nucleotide is

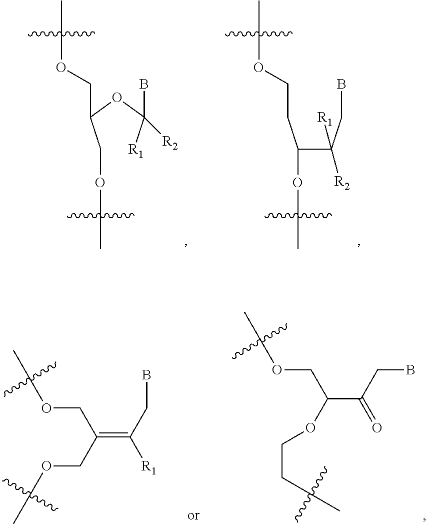

wherein B is a modified or unmodified nucleobase, R₁ and R₂ independently are H, halogen, OR₃, or alkyl; and R₃ is H, alkyl, cycloalkyl, aryl, aralkyl, heteroaryl or sugar).

Preferred sugar modifications are 2′-O-Me (2′-O-methyl), 2′-O-MOE (2′-O-methoxyethyl), 2′-F, 2′-O-[2-(methylamino)-2-oxoethyl] (2′-O-NMA), 2′-S-methyl, 2′-O—CH₂-(4′-C) (LNA), 2′-O—CH₂CH₂-(4′-C) (ENA), 2′-O-aminopropyl (2′-O-AP), 2′-O-dimethylaminoethyl (2′-O-DMAOE), 2′-O-dimethylaminopropyl (2′-O-DMAP), and 2′-O-dimethylaminoethyloxyethyl (2′-O-DMAEOE).

It is to be understood that when a particular nucleotide is linked through its 2′-position to the next nucleotide, the sugar modifications described herein can be placed at the 3′-position of the sugar for that particular nucleotide, e.g., the nucleotide that is linked through its 2′-position. A modification at the 3′ position can be present in the xylose configuration. The term “xylose configuration” refers to the placement of a substituent on the C3′ of ribose in the same configuration as the 3′-OH is in the xylose sugar.

The hydrogen attached to C4′ and/or C1′ can be replaced by a straight- or branched-optionally substituted alkyl, optionally substituted alkenyl, optionally substituted alkynyl, wherein backbone of the alkyl, alkenyl and alkynyl can contain one or more of O, S, S(O), SO₂, N(R′), C(O), N(R′)C(O)O, OC(O)N(R′), CH(R′), phosphorous containing linkage, optionally substituted aryl, optionally substituted heteroaryl, optionally substituted heterocyclic or optionally substituted cycloalkyl, where R′ is hydrogen, halogen, alkyl, alkenyl, alkynyl, alkoxy, aryl, heteroaryl, cyclyl, or heterocyclyl, each of which can be optionally substituted. In some embodiments, the hydrogen attached to the C4′ of the 5′ terminal nucleotide is replaced.

In some embodiments, C4′ and C5′ together form an optionally substituted heterocyclic, preferably comprising at least one —PX(Y)—, wherein X is H, OH, OM, SH, optionally substituted alkyl, optionally substituted alkoxy, optionally substituted alkylthio, optionally substituted alkylamino or optionally substituted dialkylamino, where M is independently for each occurrence an alki metal or transition metal with an overall charge of +1; and Y is O, S, or NR′, where R′ is hydrogen, optionally substituted aliphatic. Preferably this modification is at the 5 terminal of the oligonucleotide.

Nucleobase modifications: Adenine, cytosine, guanine, thymine and uracil are the most common bases (or nucleobases) found in nucleic acids. These bases can be modified or replaced to provide oligonucleotides having improved properties. For example, nuclease resistant oligonucleotides can be prepared with these bases or with synthetic and natural nucleobases (e.g., inosine, xanthine, hypoxanthine, nubularine, isoguanisine, or tubercidine) and any one of the above modifications. Alternatively, substituted or modified analogs of any of the above bases and “universal bases” can be employed. When a natural base is replaced by a non-natural and/or universal base, the nucleotide is said to comprise a modified nucleobase and/or a nucleobase modification herein. Modified nucleobase and/or nucleobase modifications also include natural, non-natural and universal bases, which comprise conjugated moieties, e.g. a ligand described herein. Preferred conjugate moieties for conjugation with nucleobases include cationic amino groups which can be conjugated to the nucleobase via an appropriate alkyl, alkenyl or a linker with an amide linkage.

An oligonucleotide can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine, 2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, 5-(trifluoromethyl)cytosine, 6-(azo)cytosine, N⁴-(acetyl)cytosine, 3-(3-amino-3-carboxypropyl)uracil, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole-1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouracil,4-(thio)pseudouracil,2,4-(dithio)psuedouracil,5-(alkyl)pseudouracil,5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, O⁶-substituted purines, substituted 1,2,4-triazoles, pyrrolo-pyrimidin-2-on-3-yl, 6-phenyl-pyrrolo-pyrimidin-2-on-3-yl,para-substituted-6-phenyl-pyrrolo-pyrimidin-2-on-3-yl, ortho-substituted-6-phenyl-pyrrolo-pyrimidin-2-on-3-yl,bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Alternatively, substituted or modified analogs of any of the above bases and “universal bases” can be employed.

As used herein, a universal nucleobase is any modified or nucleobase that can base pair with all of the four naturally occurring nucleobases without substantially affecting the melting behavior, recognition by intracellular enzymes or activity of the oligonucleotide duplex. Some exemplary universal nucleobases include, but are not limited to, 2,4-difluorotoluene, nitropyrrolyl, nitroindolyl, 8-aza-7-deazaadenine, 4-fluoro-6-methylbenzimidazle, 4-methylbenzimidazle, 3-methyl isocarbostyrilyl, 5-methyl isocarbostyrilyl, 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylinolyl, 4,6-dimethylindolyl, phenyl, napthalenyl, anthracenyl, phenanthracenyl, pyrenyl, stilbenyl, tetracenyl, pentacenyl, and structural derivatives thereof (see for example, Loakes, 2001, Nucleic Acids Research, 29, 2437-2447).

Further nucleobases include those disclosed in U.S. Pat. No. 3,687,808; those disclosed in International Application No. PCT/US09/038425, filed Mar. 26, 2009; those disclosed in the Concise Encyclopedia Of Polymer Science And Engineering, pages 858-859, Kroschwitz, J. I., ed. John Wiley & Sons, 1990; those disclosed by English et al., Angewandte Chemie, International Edition, 1991, 30, 613; those disclosed in Modified Nucleosides in Biochemistry, Biotechnology and Medicine, Herdewijin, P.Ed. Wiley-VCH, 2008; and those disclosed by Sanghvi, Y. S., Chapter 15, dsRNA Research and Applications, pages 289-302, Crooke, S. T. and Lebleu, B., Eds., CRC Press, 1993. Contents of all of the above are herein incorporated by reference.

Terminal modifications: In vivo applications of oligonucleotides can be limited due to presence of nucleases in the serum and/or blood. Thus in certain instances it is preferable to modify the 3′, 5′ or both ends of an oligonucleotide to make the oligonucleotide resistant against exonucleases. In some embodiments, the oligonucleotide comprises a cap structure at 3′ (3′-cap), 5′ (5′-cap) or both ends. In some embodiments, oligonucleotide comprises a 3′-cap. In another embodiment, oligonucleotide comprises a 5′-cap. In yet another embodiment, oligonucleotide comprises both a 3′ cap and a 5′ cap. It is to be understood that when an oligonucleotide comprises both a 3′ cap and a 5′ cap, such caps can be same or they can be different.

As used herein, “cap structure” refers to chemical modifications, which have been incorporated at either terminus of oligonucleotide. See for example U.S. Pat. No. 5,998,203 and International Patent Publication WO03/70918, contents of which are herein incorporated in their entireties.

Exemplary 5′-caps include, but are not limited to, ligands, 5′-5′-inverted nucleotide, 5′-5′-inverted abasic nucleotide residue, 2′-5′ linkage, 5′-amino, 5′-amino-alkyl phosphate, 5′-hexylphosphate, 5′-aminohexyl phosphate, bridging and/or non-bridging 5′-phosphoramidate, bridging and/or non-bridging 5′-phosphorothioate and/or 5′-phosphorodithioate, bridging or non bridging 5′-methylphosphonate, non-phosphodiester intersugar linkage between the end two nucleotides, 4′,5′-methylene nucleotide, I-(beta-D-erythrofuranosyl) nucleotide, 4′-thio nucleotide, carbocyclic nucleotide, 1,5-anhydrohexitol nucleotide, L-nucleotides, alpha-nucleotides, modified nucleobase nucleotide, phosphorodithioate linkage, threo-pentofuranosyl nucleotide, acyclic nucleotide, acyclic 3,4-dihydroxybutyl nucleotide, acyclic 3,5-dihydroxypentyl nucleotide, 5′-mercapto nucleotide and 5′-1,4-butanediol phosphate.

Exemplary 3′-caps include, but are not limited to, ligands, 3′-3′-inverted nucleotide, 3′-3′-inverted abasic nucleotide residue, 3′-2′-inverted nucleotide moiety, 3′-2′-inverted abasic moiety, 2′-5′-linkage, 3′-amino, 3′-amino-alkyl phosphate, 3′-hexylphosphate, 3′-aminohexyl phosphate, bridging and/or non-bridging 3′-phosphoramidate, bridging and/or non-bridging 3′-phosphorothioate and/or 3′-phosphorodithioate, bridging or non bridging 3′-methylphosphonate, non-phosphodiester intersugar linkage between the end two nucleotides, I-(beta-D-erythrofuranosyl) nucleotide, 4′-thio nucleotide, carbocyclic nucleotide, 1,5-anhydrohexitol nucleotide, L-nucleotides, alpha-nucleotides, modified nucleobase nucleotide, phosphorodithioate linkage, threo-pentofuranosyl nucleotide, acyclic nucleotide, acyclic 3,4-dihydroxybutyl nucleotide, acyclic 3,5-dihydroxypentyl nucleotide, and 3′-1,4-butanediol phosphate. For more details see Beaucage and Iyer, 1993, Tetrahedron 49, 1925, incorporated by reference herein.

Other 3′ and/or 5′ caps amenable to the invention are described in U.S. Provisional Application No. 61/223,665, filed Jul. 7, 2009, contents of which are herein incorporated in their entirety.

The 3′ and/or 5′ ends of an oligonucleotide can also be conjugated to other functional molecular entities such as labeling moieties, e.g., fluorophore (e.g., pyrene, TAMRA, fluorescein, Cy3 or Cy5 dyes) or protecting groups (based e.g., on sulfur, silicon, boron or ester). The functional molecular entities can be attached to the sugar through a phosphate group and/or a linker. The terminal atom of the linker can connect to or replace the linking atom of the phosphate group or the C-3′ or C-5′ O, N, S or C group of the sugar. Alternatively, the linker can connect to or replace the terminal atom of a nucleotide surrogate (e.g., PNAs).

Terminal modifications useful for modulating activity include modification of the 5′ end with phosphate or phosphate analogs. For example, in some embodiments the 5′ end of the oligonucleotide can be phosphorylated or includes a phosphoryl analog at the 5′ terminus. The 5′-phosphate modifications can include those which are compatible with RISC mediated gene silencing. Modifications at the 5′-terminal end can also be useful in stimulating or inhibiting the immune system of a subject. In some embodiments, the 5′-end of the oligonucleotide comprises the modification

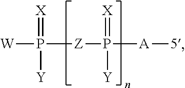

wherein W, X and Y are each independently selected from the group consisting of 0, OR (R is hydrogen, alkyl, aryl), S, Se, BR₃ (R is hydrogen, alkyl, aryl), BH₃⁻, C (i.e. an alkyl group, an aryl group, etc. . . .), H, NR₂ (R is hydrogen, alkyl, aryl), or OR (R is hydrogen, alkyl or aryl); A and Z are each independently for each occurrence absent, O, S, CH₂, NR (R is hydrogen, alkyl, aryl), or optionally substituted alkylene, wherein backbone of the alkylene can comprise one or more of O, S, SS and NR (R is hydrogen, alkyl, aryl) internally and/or at the end; and n is 0-2. In some embodiments n is 1 or 2. It is understood that A is replacing the oxygen linked to 5′ carbon of sugar.

In some embodiments, one or both hydrogen on C5′ of the 5′-terminal nucleotides can be replaced with a halogen, e.g., F.

Exemplary 5′-modificaitons include, but are not limited to, 5′-monophosphate ((HO)₂(O)P—O-5′); 5′-diphosphate ((HO)₂(O)P—O—P(HO)(O)—O-5′); 5′-triphosphate ((HO)₂(O)P—O—(HO)(O)P—O—P(HO)(O)—O-5′); 5′-monothiophosphate (phosphorothioate; (HO)2(S)P—O-5′); 5′-monodithiophosphate (phosphorodithioate; (HO)(HS)(S)P—O-5′), 5′-phosphorothiolate ((HO)2(O)P—S-5′); 5′-alpha-thiotriphosphate; 5′-beta-thiotriphosphate; 5′-gamma-thiotriphosphate; 5′-phosphoramidates ((HO)₂(O)P—NH-5′, (HO)(NH₂)(O)P—O-5′). Other 5′-modification include 5′-alkylphosphonates (R(OH)(O)P—O—5′, R=alkyl, e.g., methyl, ethyl, isopropyl, propyl, etc. . . . ), 5′-alkyletherphosphonates (R(OH)(O)P—O—5′, R=alkylether, e.g., methoxymethyl (CH₂OMe), ethoxymethyl, etc. . . . ). Other exemplary 5′-modifications include where Z is optionally substituted alkyl at least once, e.g., ((HO)₂(X)P—O[—(CH₂)ₐ—O—P(X)(OH)—O]_{b}-5′, ((HO)2(X)P—O[—(CH₂)ₐ—P(X)(OH)—O]_{b}-5′, ((HO)2(X)P—[—(CH₂)ₐ—O—P(X)(OH)—O]_{b}-5; dialkyl terminal phosphates and phosphate mimics: HO[—(CH₂)ₐ—O—P(X)(OH)—O]_{b}-5′, H₂N[—(CH₂)ₐ—O—P(X)(OH)—O]_{b}-5′, H[—(CH₂)ₐ—O—P(X)(OH)—O]_{b}-5′, Me₂N[—(CH₂)ₐ—O—P(X)(OH)—O]_{b}-5′, HO[—(CH₂)ₐ—P(X)(OH)—O]_{b}-5′, H₂N[—(CH₂)ₐ—P(X)(OH)—O]_{b}-5′, H[—(CH₂)ₐ—P(X)(OH)—O]_{b}-5′, Me₂N[—(CH₂)ₐ—P(X)(OH)—O]_{b}-5′, wherein a and b are each independently 1-10. Other embodiments include replacement of oxygen and/or sulfur with BH₃, BH₃⁻ and/or Se.

Terminal modifications can also be useful for monitoring distribution, and in such cases the preferred groups to be added include fluorophores, e.g., fluorescein or an Alexa dye, e.g., Alexa 488. Terminal modifications can also be useful for enhancing uptake, useful modifications for this include targeting ligands. Terminal modifications can also be useful for cross-linking an oligonucleotide to another moiety; modifications useful for this include mitomycin C, psoralen, and derivatives thereof.

Ligands: A wide variety of entities, e.g., ligands, can be coupled to the oligonucleotides described herein. Ligands can include naturally occurring molecules, or recombinant or synthetic molecules. Exemplary ligands include, but are not limited to, polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG, e.g., PEG-2K, PEG-5K, PEG-10K, PEG-12K, PEG-15K, PEG-20K, PEG-40K), MPEG, [MPEG]₂, polyvinyl alcohol (PVA), polyurethane, poly(2-ethylacryllic acid), N-isopropylacrylamide polymers, polyphosphazine, polyethylenimine, cationic groups, spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, thyrotropin, melanotropin, lectin, glycoprotein, surfactant protein A, mucin, glycosylated polyaminoacids, transferrin, bisphosphonate, polyglutamate, polyaspartate, aptamer, asialofetuin, hyaluronan, procollagen, immunoglobulins (e.g., antibodies), insulin, transferrin, albumin, sugar-albumin conjugates, intercalating agents (e.g., acridines), cross-linkers (e.g. psoralen, mitomycin C), porphyrins (e.g., TPPC4, texaphyrin, Sapphyrin), polycyclic aromatic hydrocarbons (e.g., phenazine, dihydrophenazine), artificial endonucleases (e.g., EDTA), lipophilic molecules (e.g, steroids, bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), peptides (e.g., an alpha helical peptide, amphipathic peptide, RGD peptide, cell permeation peptide, endosomolytic/fusogenic peptide), alkylating agents, phosphate, amino, mercapto, polyamino, alkyl, substituted alkyl, radiolabeled markers, enzymes, haptens (e.g. biotin), transport/absorption facilitators (e.g., naproxen, aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, AP, antibodies, hormones and hormone receptors, lectins, carbohydrates, multivalent carbohydrates, vitamins (e.g., vitamin A, vitamin E, vitamin K, vitamin B, e.g., folic acid, B12, riboflavin, biotin and pyridoxal), vitamin cofactors, lipopolysaccharide, an activator of p38 MAP kinase, an activator of NF-κB, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, natural or recombinant low density lipoprotein (LDL), natural or recombinant high-density lipoprotein (HDL), and a cell-permeation agent (e.g., a.helical cell-permeation agent).

Peptide and peptidomimetic ligands include those having naturally occurring or modified peptides, e.g., D or L peptides; α, β, or γ peptides; N-methyl peptides; azapeptides; peptides having one or more amide, i.e., peptide, linkages replaced with one or more urea, thiourea, carbamate, or sulfonyl urea linkages; or cyclic peptides. A peptidomimetic (also referred to herein as an oligopeptidomimetic) is a molecule capable of folding into a defined three-dimensional structure similar to a natural peptide. The peptide or peptidomimetic ligand can be about 5-50 amino acids long, e.g., about 5, 10, 15, 20, 25, 30, 35, 40, 45, or 50 amino acids long.

Exemplary amphipathic peptides include, but are not limited to, cecropins, lycotoxins, paradaxins, buforin, CPF, bombinin-like peptide (BLP), cathelicidins, ceratotoxins, S. clava peptides, hagfish intestinal antimicrobial peptides (HFIAPs), magainines, brevinins-2, dermaseptins, melittins, pleurocidin, H₂A peptides, Xenopus peptides, esculentinis-1, and caerins.

As used herein, the term “endosomolytic ligand” refers to molecules having endosomolytic properties. Endosomolytic ligands promote the lysis of and/or transport of the composition of the invention, or its components, from the cellular compartments such as the endosome, lysosome, endoplasmic reticulum (ER), golgi apparatus, microtubule, peroxisome, or other vesicular bodies within the cell, to the cytoplasm of the cell. Some exemplary endosomolytic ligands include, but are not limited to, imidazoles, poly or oligoimidazoles, linear or branched polyethyleneimines (PEIs), linear and brached polyamines, e.g. spermine, cationic linear and branched polyamines, polycarboxylates, polycations, masked oligo or poly cations or anions, acetals, polyacetals, ketals/polyketals, orthoesters, linear or branched polymers with masked or unmasked cationic or anionic charges, dendrimers with masked or unmasked cationic or anionic charges, polyanionic peptides, polyanionic peptidomimetics, pH-sensitive peptides, natural and synthetic fusogenic lipids, natural and synthetic cationic lipids.

Exemplary endosomolytic/fusogenic peptides include, but are not limited to,

SEQ ID NO: 16,
AALEALAEALEALAEALEALAEAAAAGGC (GALA);
SEQ ID NO: 17,
AALAEALAEALAEALAEALAEALAAAAGGC (EALA);
SEQ ID NO: 18,
ALEALAEALEALAEA;
SEQ ID NO: 19,
GLFEAIEGFIENGWEGMIWDYG (INF-7);
SEQ ID NO: 20,
GLFGAIAGFIENGWEGMIDGWYG (Inf HA-2);
SEQ ID NO: 21,
GLFEAIEGFIENGWEGMIDGWYGCGLFEAIEGFIENGWEGMID GWYGC
(diINF-7);
SEQ ID NO: 22,
GLFEAIEGFIENGWEGMIDGGCGLFEAIEGFIENGWEGMIDGGC
(diINF-3);
SEQ ID NO: 23,
GLFGALAEALAEALAEHLAEALAEALEALAAGGSC (GLF);
SEQ ID NO: 24,
GLFEAIEGFIENGWEGLAEALAEALEALAAGGSC (GALA-INF3);
SEQ ID NO: 25,
GLF EAI EGFI ENGW EGnI DG K GLF EAI EGFI ENGW EGnI
DG (INF-5, n is norleucine);
SEQ ID NO: 26,
LFEALLELLESLWELLLEA (JTS-1);
SEQ ID NO: 27,
GLFKALLKLLKSLWKLLLKA (ppTG1);
SEQ ID NO: 28,
GLFRALLRLLRSLWRLLLRA (ppTG20);
SEQ ID NO: 29,
WEAKLAKALAKALAKHLAKALAKALKACEA (KALA);
SEQ ID NO: 30,
GLFFEAIAEFIEGGWEGLIEGC (HA);
SEQ ID NO: 31,
GIGAVLKVLTTGLPALISWIKRKRQQ (Melittin);
SEQ ID NO: 32,
H₅WYG;
and
SEQ ID NO: 33,
CHK₆HC.

Without wishing to be bound by theory, fusogenic lipids fuse with and consequently destabilize a membrane. Fusogenic lipids usually have small head groups and unsaturated acyl chains. Exemplary fusogenic lipids include, but are not limited to, 1,2-dileoyl-sn-3-phosphoethanolamine (DOPE), phosphatidylethanolamine (POPE), palmitoyloleoylphosphatidylcholine (POPC), (6Z,9Z,28Z,31Z)-heptatriaconta-6,9,28,31-tetraen-19-ol (Di-Lin), N-methyl(2,2-di((9Z,12Z)-octadeca-9,12-dienyl)-1,3-dioxolan-4-yl)methanamine (DLin-k-DMA) and N-methyl-2-(2,2-di((9Z,12Z)-octadeca-9,12-dienyl)-1,3-dioxolan-4-yl)ethanamine.

Synthetic polymers with endosomolytic activity amenable to the present invention are described in U.S. Pat. App. Pub. Nos. 2009/0048410; 2009/0023890; 2008/0287630; 2008/0287628; 2008/0281044; 2008/0281041; 2008/0269450; 2007/0105804; 2007/0036865; and 2004/0198687, content of all of is incorporated herein by reference in its entirety.

Exemplary cell permeation peptides include, but are not limited to,

SEQ ID NO: 34,
RQIKIWFQNRRMKWKK (penetratin);
SEQ ID NO: 35,
GRKKRRQRRRPPQC (Tat fragment 48-60);
SEQ ID NO: 36,
GALFLGWLGAAGSTMGAWSQPKKKRKV (signal sequence based
peptide);
SEQ ID NO: 37,
LLIILRRRIRKQAHAHSK (PVEC);
SEQ ID NO: 38,
GWTLNSAGYLLKINLKALAALAKKIL (transportan);
SEQ ID NO: 39,
KLALKLALKALKAALKLA (amphiphilic model peptide);
SEQ ID NO: 40,
RRRRRRRRR (Arg9);
SEQ ID NO: 41,
KFFKFFKFFK (Bacterial cell wall permeating
peptide);
SEQ ID NO: 42,
LLGDFFRKSKEKIGKEFKRIVQRIKDFLRNLVPRTES (LL-37);
SEQ ID NO: 43,
SWLSKTAKKLENSAKKRISEGIAIAIQGGPR (cecropin P1);
SEQ ID NO: 44,
ACYCRIPACIAGERRYGTCIYQGRLWAFCC (α-defensin)
SEQ ID NO: 45,
DHYNCVSSGGQCLYSACPIFTKIQGTCYRGKAKCCK
(β-defensin);
SEQ ID NO: 46,
RRRPRPPYLPRPRPPPFFPPRLPPRIPPGFPPRFPPRFPGKR-NH2
(PR-39);
SEQ ID NO: 47,
ILPWKWPWWPWRR-NH2 (indolicidin);
SEQ ID NO: 48,
AAVALLPAVLLALLAP (RFGF);
SEQ ID NO: 49,
AALLPVLLAAP (RFGF analogue);
and
SEQ ID NO: 50,
RKCRIVVIRVCR (bactenecin).

Exemplary cationic groups include, but are not limited to, protonated amino groups, derived from e.g., O-AMINE (AMINE=NH₂; alkylamino, dialkylamino, heterocyclyl, arylamino, diaryl amino, heteroaryl amino, or diheteroaryl amino, ethylene diamine, polyamino); aminoalkoxy, e.g., O(CH₂)ₙAMINE, (e.g., AMINE=NH₂; alkylamino, dialkylamino, heterocyclyl, arylamino, diaryl amino, heteroaryl amino, or diheteroaryl amino, ethylene diamine, polyamino); amino (e.g. NH₂; alkylamino, dialkylamino, heterocyclyl, arylamino, diaryl amino, heteroaryl amino, diheteroaryl amino, or amino acid); and NH(CH₂CH₂NH)ₙCH₂CH₂-AMINE (AMINE=NH₂; alkylamino, dialkylamino, heterocyclyl, arylamino, diaryl amino, heteroaryl amino, or diheteroaryl amino).

As used herein the term “targeting ligand” refers to any molecule that provides an enhanced affinity for a selected target, e.g., a cell, cell type, tissue, organ, region of the body, or a compartment, e.g., a cellular, tissue or organ compartment. Some exemplary targeting ligands include, but are not limited to, antibodies, antigens, folates, receptor ligands, carbohydrates, aptamers, integrin receptor ligands, chemokine receptor ligands, transferrin, biotin, serotonin receptor ligands, PSMA, endothelin, GCPII, somatostatin, LDL and HDL ligands.

Carbohydrate based targeting ligands include, but are not limited to, D-galactose, multivalent galactose, N-acetyl-D-galactose (GalNAc), multivalent GalNAc, e.g. GalNAc2 and GalNAc3; D-mannose, multivalent mannose, multivalent lactose, N-acetyl-galactosamine, N-acetyl-gulucosamine, multivalent fucose, glycosylated polyaminoacids and lectins. The term multivalent indicates that more than one monosaccharide unit is present. Such monosaccharide subunits can be linked to each other through glycosidic linkages or linked to a scaffold molecule.

A number of folate and folate analogs amenable to the present invention as ligands are described in U.S. Pat. Nos. 2,816,110; 51410,104; 5,552,545; 6,335,434 and 7,128,893, contents of which are herein incorporated in their entireties by reference.

As used herein, the terms “PK modulating ligand” and “PK modulator” refers to molecules which can modulate the pharmacokinetics of the oligoncucleotide. Some exemplary PK modulator include, but are not limited to, lipophilic molecules, bile acids, sterols, phospholipid analogues, peptides, protein binding agents, vitamins, fatty acids, phenoxazine, aspirin, naproxen, ibuprofen, suprofen, ketoprofen, (S)-(+)-pranoprofen, carprofen, PEGs, biotin, and transthyretia-binding ligands (e.g., tetraiidothyroacetic acid, 2,4,6-triiodophenol and flufenamic acid). Oligonucleotides that comprise a number of phosphorothioate intersugar linkages are also known to bind to serum protein, thus short oligonucleotides, e.g. oligonucleotides of comprising from about 5 to 30 nucleotides (e.g., 5 to 25 nulceotides, preferably 5 to 20 nucleotides, e.g., 5, 6, 7, 8, 9, 10, 11, 12, 13, 14, 15, 16, 17, 18, 19, or 20 nucleotides), and that comprise a plurality of phosphorothioate linkages in the backbone are also amenable to the present invention as ligands (e.g. as PK modulating ligands). The PK modulating oligonucleotide can comprise at least 3, 4, 5, 6, 7, 8, 9, 10, 11, 12, 13, 14, 15 or more phosphorothioate and/or phosphorodithioate linkages. In some embodiments, all internucleotide linkages in PK modulating oligonucleotide are phosphorothioate and/or phosphorodithioates linkages. In addition, aptamers that bind serum components (e.g. serum proteins) are also amenable to the present invention as PK modulating ligands. Binding to serum components (e.g. serum proteins) can be predicted from albumin binding assays, such as those described in Oravcova, et al., Journal of Chromatography B (1996), 677: 1-27.

When two or more ligands are present, the ligands can all have same properties, all have different properties or some ligands have the same properties while others have different properties. For example, a ligand can have targeting properties, have endosomolytic activity or have PK modulating properties. In a preferred embodiment, all the ligands have different properties.

Ligands can be coupled to the oligonucleotides at various places, for example, 3′-end, 5′-end, and/or at an internal position. When two or more ligands are present, the ligand can be on opposite ends of an oligonucleotide. In preferred embodiments, the ligand is attached to the oligonucleotides via an intervening tether/linker. The ligand or tethered ligand can be present on a monomer when said monomer is incorporated into the growing strand. In some embodiments, the ligand can be incorporated via coupling to a “precursor” monomer after said “precursor” monomer has been incorporated into the growing strand. For example, a monomer having, e.g., an amino-terminated tether (i.e., having no associated ligand), e.g., monomer-linker-NH₂ can be incorporated into a growing oligonucleotide strand. In a subsequent operation, i.e., after incorporation of the precursor monomer into the strand, a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction can be incorporated e.g., an azide or alkyne terminated tether/linker. In a subsequent operation, i.e., after incorporation of the precursor monomer into the strand, a ligand having complementary chemical group, e.g. an alkyne or azide can be attached to the precursor monomer by coupling the alkyne and the azide together.

For double-stranded oligonucleotides, ligands can be attached to one or both strands.

In some embodiments, ligand can be conjugated to nucleobases, sugar moieties, or internucleosidic linkages of nucleic acid molecules. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. When a ligand is conjugated to a nucleobase, the preferred position is one that does not interfere with hybridization, i.e., does not interfere with the hydrogen bonding interactions needed for base pairing.

Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Internucleosidic linkages can also bear conjugate moieties. For phosphorus-containing linkages (e.g., phosphodiester, phosphorothioate, phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

There are numerous methods for preparing conjugates of oligomeric compounds. Generally, an oligomeric compound is attached to a conjugate moiety by contacting a reactive group (e.g., OH, SH, amine, carboxyl, aldehyde, and the like) on the oligomeric compound with a reactive group on the conjugate moiety. In some embodiments, one reactive group is electrophilic and the other is nucleophilic.

For example, an electrophilic group can be a carbonyl-containing functionality and a nucleophilic group can be an amine or thiol. Methods for conjugation of nucleic acids and related oligomeric compounds with and without linking groups are well described in the literature such as, for example, in Manoharan in Antisense Research and Applications, Crooke and LeBleu, eds., CRC Press, Boca Raton, Fla., 1993, Chapter 17, which is incorporated herein by reference in its entirety.

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,149,782; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928; 5,672,662; 5,688,941; 5,714,166; 6,153,737; 6,172,208; 6,300,319; 6,335,434; 6,335,437; 6,395,437; 6,444,806; 6,486,308; 6,525,031; 6,528,631; 6,559,279; content all of which is incorporated by reference in its entirety.

Ligand carriers: In some embodiments, the ligands, e.g. endosomolytic ligands, targeting ligands or other ligands, are linked to a monomer which is then incorporated into the growing oligonucleotide strand during chemical synthesis. Such monomers are also referred to as carrier monomers herein. The carrier monomer is a cyclic group or acyclic group; preferably, the cyclic group is selected from the group consisting of pyrrolidinyl, pyrazolinyl, pyrazolidinyl, imidazolinyl, imidazolidinyl, piperidinyl, piperazinyl, [1,3]-dioxolane, oxazolidinyl, isoxazolidinyl, morpholinyl, thiazolidinyl, isothiazolidinyl, quinoxalinyl, pyridazinonyl, tetrahydrofuryl and decalin; preferably, the acyclic group is selected from serinol backbone or diethanolamine backbone. In some embodiments, the cyclic carrier monomer is based on pyrrolidinyl such as 4-hydroxyproline or a derivative thereof.

Exemplary ligands and ligand conjugated monomers amenable to the invention are described in U.S. patent application Ser. No. 10/916,185, filed Aug. 10, 2004; Ser. No. 10/946,873, filed Sep. 21, 2004; Ser. No. 10/985,426, filed Nov. 9, 2004; Ser. No. 10/833,934, filed Aug. 3, 2007; Ser. No. 11/115,989 filed Apr. 27, 2005, Ser. No. 11/119,533, filed Apr. 29, 2005; Ser. No. 11/197,753, filed Aug. 4, 2005; Ser. No. 11/944,227, filed Nov. 21, 2007; Ser. No. 12/328,528, filed Dec. 4, 2008; and Ser. No. 12/328,537, filed Dec. 4, 2008, contents which are herein incorporated in their entireties by reference for all purposes. Ligands and ligand conjugated monomers amenable to the invention are also described in International Application Nos. PCT/US04/001461, filed Jan. 21, 2004; PCT/US04/010586, filed Apr. 5, 2004; PCT/US04/011255, filed Apr. 9, 2005; PCT/US05/014472, filed Apr. 27, 2005; PCT/US05/015305, filed Apr. 29, 2005; PCT/US05/027722, filed Aug. 4, 2005; PCT/US08/061289, filed Apr. 23, 2008; PCT/US08/071576, filed Jul. 30, 2008; PCT/US08/085574, filed Dec. 4, 2008 and PCT/US09/40274, filed Apr. 10, 2009, contents which are herein incorporated in their entireties by reference for all purposes.

Linkers: In some embodiments, the covalent linkages between the oligonucleotide and other components, e.g. a ligand or a ligand carrying monomer can be mediated by a linker. This linker can be cleavable linker or non-cleavable linker, depending on the application. As used herein, a “cleavable linker” refers to linkers that are capable of cleavage under various conditions. Conditions suitable for cleavage can include, but are not limited to, pH, UV irradiation, enzymatic activity, temperature, hydrolysis, elimination and substitution reactions, redox reactions, and thermodynamic properties of the linkage. In some embodiments, a cleavable linker can be used to release the oligonucleotide after transport to the desired target. The intended nature of the conjugation or coupling interaction, or the desired biological effect, will determine the choice of linker group.

As used herein, the term “linker” means an organic moiety that connects two parts of a compound. Linkers typically comprise a direct bond or an atom such as oxygen or sulfur, a unit such as NR¹, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, where one or more methylenes can be interrupted or terminated by O, S, S(O), SO₂, N(R¹)₂, C(O), cleavable linking group, substituted or unsubstituted aryl, substituted or unsubstituted heteroaryl, substituted or unsubstituted heterocyclic; where R′ is hydrogen, acyl, aliphatic or substituted aliphatic.

In some embodiments, the linker is a branched linker. The branchpoint of the branched linker may be at least trivalent, but can be a tetravalent, pentavalent or hexavalent atom, or a group presenting such multiple valencies. In some embodiments, the branchpoint is —N, —N(Q)-C, —O—C, —S—C, —SS—C, —C(O)N(Q)-C, —OC(O)N(Q)-C, —N(Q)C(O)—C, or —N(Q)C(O)O—C; wherein Q is independently for each occurrence H or optionally substituted alkyl. In some embodiments, the branchpoint is glycerol or derivative thereof.

Cleavable Linking Groups: A cleavable linking group is one which is sufficiently stable outside the cell, but which upon entry into a target cell is cleaved to release the two parts the linker is holding together. In a preferred embodiment, the cleavable linking group is cleaved at least 10 times or more, preferably at least 100 times faster in the target cell or under a first reference condition (which can, e.g., be selected to mimic or represent intracellular conditions) than in the blood or serum of a subject, or under a second reference condition (which can, e.g., be selected to mimic or represent conditions found in the blood or serum).

Cleavable linking groups are susceptible to cleavage agents, e.g., pH, redox potential or the presence of degradative molecules. Generally, cleavage agents are more prevalent or found at higher levels or activities inside cells than in serum or blood. Examples of such degradative agents include: redox agents which are selected for particular substrates or which have no substrate specificity, including, e.g., oxidative or reductive enzymes or reductive agents such as mercaptans, present in cells, that can degrade a redox cleavable linking group by reduction; esterases; amidases; endosomes or agents that can create an acidic environment, e.g., those that result in a pH of five or lower; enzymes that can hydrolyze or degrade an acid cleavable linking group by acting as a general acid, peptidases (which can be substrate specific) and proteases, and phosphatases.

A linker can include a cleavable linking group that is cleavable by a particular enzyme. The type of cleavable linking group incorporated into a linker can depend on the cell to be targeted. For example, liver targeting ligands can be linked to the cationic lipids through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In some embodiments, cleavable linking group is cleaved at least 1.25, 1.5, 1.75, 2, 3, 4, 5, 10, 25, 50, or 100 times faster in the cell (or under in vitro conditions selected to mimic intracellular conditions) as compared to blood or serum (or under in vitro conditions selected to mimic extracellular conditions). In some embodiments, the cleavable linking group is cleaved by less than 90%, 80%, 70%, 60%, 50%, 40%, 30%, 20%, 10%, 5%, or 1% in the blood (or in vitro conditions selected to mimic extracellular conditions) as compared to in the cell (or under in vitro conditions selected to mimic intracellular conditions)

Exemplary cleavable linking groups include, but are not limited to, redox cleavable linking groups (e.g., —S—S— and —C(R)₂—S—S—, wherein R is H or C₁-C₆ alkyl and at least one R is C₁-C₆ alkyl such as CH₃ or CH₂CH₃); phosphate-based cleavable linking groups (e.g., —O—P(O)(OR)—O—, —O—P(S)(OR)—O—, —O—P(S)(SR)—O—, —S—P(O)(OR)—O—, —O—P(O)(OR—S—, —S—P(O)(OR—S—, —O—P(S)(ORk)-S—, —S—P(S)(OR)—O—, —O—P(O)(R)—O—, —O—P(S)(R)—O—, —S—P(O)(R)—O—, —S—P(S)(R)—O—, —S—P(O)(R)—S—, —O—P(S)(R)—S—, —O—P(O)(OH)—O—, —O—P(S)(OH)—O—, —O—P(S)(SH)—O—, —S—P(O)(OH)—O—, —O—P(O)(OH)—S—, —S—P(O)(OH)—S—, —O—P(S)(OH)—S—, —S—P(S)(OH)—O—, —O—P(O)(H)—O—, —O—P(S)(H)—O—, —S—P(O)(H)—O—, —S—P(S)(H)—O—, —S—P(O)(H)—S—, and —O—P(S)(H)—S—, wherein R is optionally substituted linear or branched C₁-C₁₀ alkyl); acid celavable linking groups (e.g., hydrazones, esters, and esters of amino acids, —C═NN— and —OC(O)—); ester-based cleavable linking groups (e.g., —C(O)O—); peptide-based cleavable linking groups, (e.g., linking groups that are cleaved by enzymes such as peptidases and proteases in cells, e.g., —NHCHR^{A}C(O)NHCHR^{B}C(O)—, where R^{A} and R^{B} are the R groups of the two adjacent amino acids). A peptide based cleavable linking group comprises two or more amino acids. In some embodiments, the peptide-based cleavage linkage comprises the amino acid sequence that is the substrate for a peptidase or a protease found in cells.

In some embodiments, an acid cleavable linking group is cleaveable in an acidic environment with a pH of about 6.5 or lower (e.g., about 6.5, 6.0, 5.5, 5.0, or lower), or by agents such as enzymes that can act as a general acid.

General References: General references for oligonucleotide modification are discussed below. In some embodiments, RNAi agents that inhibit SH2B3 and oligonucleotides used in accordance with this invention can be synthesized with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (especially Chapter 1, Modern machine-aided methods of oligodeoxyribonucleotide synthesis, Chapter 2, Oligoribonucleotide synthesis, Chapter 3, 2′-O-Methyloligoribonucleotides: synthesis and applications, Chapter 4, Phosphorothioate oligonucleotides, Chapter 5, Synthesis of oligonucleotide phosphorodithioates, Chapter 6, Synthesis of oligo-2′-deoxyribonucleoside methylphosphonates, and. Chapter 7, Oligodeoxynucleotides containing modified bases. Other particularly useful synthetic procedures, reagents, blocking groups and reaction conditions are described in Martin, P., Helv. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein. Modification described in WO 00/44895, WO01/75164, or WO02/44321 can be used herein. The disclosure of all publications, patents, and published patent applications listed herein are hereby incorporated by reference.

Modification of the Phosphate Group References: The preparation of phosphinate oligonucleotides is described in U.S. Pat. No. 5,508,270. The preparation of alkyl phosphonate oligonucleotides is described in U.S. Pat. No. 4,469,863. The preparation of phosphoramidite oligonucleotides is described in U.S. Pat. No. 5,256,775 or U.S. Pat. No. 5,366,878. The preparation of phosphotriester oligonucleotides is described in U.S. Pat. No. 5,023,243. The preparation of boranophosphate oligonucleotide is described in U.S. Pat. Nos. 5,130,302 and 5,177,198. The preparation of 3′-Deoxy-3′-amino phosphoramidate oligonucleotides is described in U.S. Pat. No. 5,476,925. 3′-Deoxy-3′-methylenephosphonate oligonucleotides is described in An, H, et al. J. Org. Chem. 2001, 66, 2789-2801. Preparation of sulfur bridged nucleotides is described in Sproat et al. Nucleosides Nucleotides 1988, 7,651 and Crosstick et al. Tetrahedron Lett. 1989, 30, 4693.

Replacement of the Phosphate Group References:_Methylenemethylimino linked oligonucleosides, also identified herein as MMI linked oligonucleosides, methylenedimethylhydrazo linked oligonucleosides, also identified herein as MDH linked oligonucleosides, and methylenecarbonylamino linked oligonucleosides, also identified herein as amide-3 linked oligonucleosides, and methyleneaminocarbonyl linked oligonucleosides, also identified herein as amide-4 linked oligonucleosides as well as mixed intersugar linkage compounds having, as for instance, alternating MMI and PO or PS linkages can be prepared as is described in U.S. Pat. Nos. 5,378,825, 5,386,023, 5,489,677 and in International Application Nos. PCT/US92/04294 and PCT/US92/04305 (published as WO 92/20822 WO and 92/20823, respectively). Formacetal and thioformacetal linked oligonucleosides can be prepared as is described in U.S. Pat. Nos. 5,264,562 and 5,264,564. Ethylene oxide linked oligonucleosides can be prepared as is described in U.S. Pat. No. 5,223,618. Siloxane replacements are described in Cormier, J. F. et al. Nucleic Acids Res. 1988, 16, 4583. Carbonate replacements are described in Tittensor, J. R. J. Chem. Soc. C 1971, 1933. Carboxymethyl replacements are described in Edge, M. D. et al. J. Chem. Soc. Perkin Trans. 1 1972, 1991. Carbamate replacements are described in Stirchak, E. P. Nucleic Acids Res. 1989, 17, 6129.

Replacement of the Phosphate-Ribose Backbone References: Cyclobutyl sugar surrogate compounds can be prepared as is described in U.S. Pat. No. 5,359,044. Pyrrolidine sugar surrogate can be prepared as is described in U.S. Pat. No. 5,519,134. Morpholino sugar surrogates can be prepared as is described in U.S. Pat. Nos. 5,142,047 and 5,235,033, and other related patent disclosures. Peptide Nucleic Acids (PNAs) are known per se and can be prepared in accordance with any of the various procedures referred to in Peptide Nucleic Acids (PNA): Synthesis, Properties and Potential Applications, Bioorganic & Medicinal Chemistry, 1996, 4, 5-23. They can also be prepared in accordance with U.S. Pat. No. 5,539,083.

Sugar Modification References: Modifications to the 2′ modifications can be found in Verma, S. et al. Annu. Rev. Biochem. 1998, 67, 99-134 and all references therein. Specific modifications to the ribose can be found in the following references: 2′-fluoro (Kawasaki et. al., J. Med. Chem., 1993, 36, 831-841), 2′-MOE (Martin, P. Helv. Chim. Acta 1996, 79, 1930-1938), “LNA” (Wengel, J. Acc. Chem. Res. 1999, 32, 301-310).

Modifications of Nucleobases References: Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,457,191; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference in its entirety.

Terminal Modification References: Terminal modifications are described in Manoharan, M. et al. Antisense and Nucleic Acid Drug Development 12, 103-128 (2002) and references therein.

Placement of Modifications within an Oligonucleotide (e.g., Placement within an RNAi Agents that Inhibits SH2B3)

As oligonucleotides, such as RNAi agents that inhibit SH2B3 can be are polymers of subunits or monomers, many of the modifications described herein can occur at a position which is repeated within an oligonucleotide, e.g., a modification of a nucleobase, a sugar, a phosphate moiety, or the non-bridging oxygen of a phosphate moiety. It is not necessary for all positions in a given oligonucleotide to be uniformly modified, and in fact more than one of the aforementioned modifications can be incorporated in a single oligonucleotide or even at a single nucleoside within an oligonucleotide.

In some cases the modification will occur at all of the subject positions in the oligonucleotide but in many, and in fact in most cases it will not. By way of example, a modification can occur at a 3′ or 5′ terminus position, can occur in the internal region, can occur in 3′, 5′ or both terminal regions, e.g. at a position on a termus nucleotide or in the last 2, 3, 4, 5, 6, 7, 8, 9, or 10 nucleotides of an oligonucleotide. In some embodiments, the terminus nucleotide does not comprise a modification.

In some embodiments, the terminus nucleotide or the last 2, 3, 4, 5, 6, 7, 8, 9, or 10 nucleotides of at least one end of the oligonucleotide all comprise at least one modification. In some embodiments, the modification is same. In some embodiments, the terminus nucleotide or the last 2, 3, 4, 5, 6, 7, 8, 9, or 10 nucleotides at both ends of the oligonucleotide all comprise at least one modification. It is to be understood that type of modification and number of modified nucleotides on one end of the oligonucleotide is independent of type of modification and number of modified nucleotides on the other end of the oligonucleotide.

When the oligonucleotide is double-stranded or partially double-stranded, a modification can occur in the double strand region, the single strand region, or in both the double- and single-stranded regions. In some embodiments, a modification described herein does not occur in the region corresponding to the target cleavage site region. For example, a phosphorothioate modification at a non-bridging oxygen position can occur at one or both termini, can occur in a terminal regions, e.g., at a position on a terminal nucleotide or in the last 2, 3, 4, 5, 6, 7, 8, 9, or 10 nucleotides of a strand, or can occur in double strand and single strand regions, particularly at termini.

Some modifications can preferably be included on an oligonucleotide at a particular location, e.g., at an internal position of a strand, or on the 5′ or 3′ end of an oligonucleotide. A preferred location of a modification on an oligonucleotide, can confer preferred properties on the oligonucleotide. For example, preferred locations of particular modifications can confer increased resistance to endonuclease or exonuclease activity.

Production of RNAi Agents that Inhibit SH2B3 and Mimetics Thereof.

In some embodiments, RNAi agents that inhibit SH2B3 as disclosed herien can be prepared using solution-phase or solid-phase organic synthesis, or enzymatically by methods known in the art. Organic synthesis offers the advantage that the oligonucleotide strands comprising non-natural or modified nucleotides can be easily prepared. Any other means for such synthesis known in the art can additionally or alternatively be employed. It is also known to use similar techniques to prepare other oligonucleotides, such as the phosphorothioates, phosphorodithioates and alkylated derivatives. The double-stranded oligonucleotide compounds of the invention can be prepared using a two-step procedure. First, the individual strands of the double-stranded molecule are prepared separately. Then, the component strands are annealed.

MicroRNAs have also been implicated in modulation of pathogens in hosts. For example, see Jopling, C. L., et al., Science (2005) vol. 309, pp 1577-1581.

Regardless of the method of synthesis, the oligonucleotide can be prepared in a solution (e.g., an aqueous and/or organic solution) that is appropriate for formulation. For example, the oligonucleotide preparation can be precipitated and redissolved in pure double-distilled water, and lyophilized. The dried oligonucleotide can then be resuspended in a solution appropriate for the intended formulation process.

Teachings regarding the synthesis of particular modified oligonucleotides can be found in the following U.S. patents or pending patent applications: U.S. Pat. Nos. 5,138,045 and 5,218,105, drawn to polyamine conjugated oligonucleotides; U.S. Pat. No. 5,212,295, drawn to monomers for the preparation of oligonucleotides having chiral phosphorus linkages; U.S. Pat. Nos. 5,378,825 and 5,541,307, drawn to oligonucleotides having modified backbones; U.S. Pat. No. 5,386,023, drawn to backbone-modified oligonucleotides and the preparation thereof through reductive coupling; U.S. Pat. No. 5,457,191, drawn to modified nucleobases based on the 3-deazapurine ring system and methods of synthesis thereof; U.S. Pat. No. 5,459,255, drawn to modified nucleobases based on N-2 substituted purines; U.S. Pat. No. 5,521,302, drawn to processes for preparing oligonucleotides having chiral phosphorus linkages; U.S. Pat. No. 5,539,082, drawn to peptide nucleic acids; U.S. Pat. No. 5,554,746, drawn to oligonucleotides having beta-lactam backbones; U.S. Pat. No. 5,571,902, drawn to methods and materials for the synthesis of oligonucleotides; U.S. Pat. No. 5,578,718, drawn to nucleosides having alkylthio groups, wherein such groups can be used as linkers to other moieties attached at any of a variety of positions of the nucleoside; U.S. Pat. Nos. 5,587,361 and 5,599,797, drawn to oligonucleotides having phosphorothioate linkages of high chiral purity; U.S. Pat. No. 5,506,351, drawn to processes for the preparation of 2′-O-alkyl guanosine and related compounds, including 2,6-diaminopurine compounds; U.S. Pat. No. 5,587,469, drawn to oligonucleotides having N-2 substituted purines; U.S. Pat. No. 5,587,470, drawn to oligonucleotides having 3-deazapurines; U.S. Pat. No. 5,223,168, and U.S. Pat. No. 5,608,046, both drawn to conjugated 4′-desmethyl nucleoside analogs; U.S. Pat. Nos. 5,602,240, and 5,610,289, drawn to backbone-modified oligonucleotide analogs; and U.S. Pat. Nos. 6,262,241, and 5,459,255, drawn to, inter alia, methods of synthesizing 2′-fluoro-oligonucleotides.

Additionally, in some embodimetns, RNAi agents that inhibit SH2B3 can be recombinantly produced, or synthesized in vitro by a variety of techniques well known to one of ordinary skill in the art.

RNAi agents that inhibit SH2B3 can be obtained by preparing a recombinant version thereof (i.e., by using the techniques of genetic engineering to produce a recombinant nucleic acid which can then be isolated or purified by techniques well known to one of ordinary skill in the art). This approach involves growing a culture of host cells in a suitable culture medium, and purifying the RNAi agents that inhibit SH2B3 from the cells or the culture in which the cells are grown. For example, the methods include a process for producing a RNAi agents that inhibit SH2B3 in which a host cell, containing a suitable expression vector that includes a nucleic acid encoding a RNAi agent that inhibits SH2B3, is cultured under conditions that allow expression of the encoded RNAi agent. The RNAi can be recovered from the culture, from the culture medium or from a lysate prepared from the host cells, and further purified. The host cell can be a higher eukaryotic host cell such as a mammalian cell, a lower eukaryotic host cell such as a yeast cell, or the host cell can be a prokaryotic cell such as a bacterial cell. Introduction of a vector containing the nucleic acid encoding the RNAi agent into the host cell can be effected by calcium phosphate transfection, DEAE-dextran mediated transfection, or electroporation (Davis, L. et al., Basic Methods in Molecular Biology (1986)). Such a method is useful, for example, where the RNAi agent is a miRNA or the like.

Any host/vector system can be used to express one or more RNAi agents that inhibits SH2B3. These include, but are not limited to, eukaryotic hosts such as HeLa cells and yeast, as well as prokaryotic host such as E. coli and B. subtilis. An RNAi agents that inhibits SH2B3, e.g., miRNA that inhibits SH2B3 is under the control of an appropriate promoter. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989). In the preferred embodiment, a RNAi agent that inhibits SH2B3, e.g., miRNA is expressed in mammalian cells. Examples of mammalian expression systems include C127, monkey COS cells, Chinese Hamster Ovary (CHO) cells, human kidney 293 cells, human epidermal A43 1 cells, human Colo205 cells, 3T3 cells, CV-1 cells, other transformed primate cell lines, normal diploid cells, cell strains derived from in vitro culture of primary tissue, primary explants, HeLa cells, mouse L cells, BILK, HL-60, U937, HaK or Jurkat cells.

Mammalian expression vectors will comprise an origin of replication, a suitable promoter, polyadenylation site, transcriptional termination sequences, and 5′ flanking non-transcribed sequences. DNA sequences derived from the SV40 viral genome, for example, SV40 origin, early promoter, enhancer, splice, and polyadenylation sites may be used to provide the required non-transcribed genetic elements. Potentially suitable yeast strains include Saccharomyces cerevsiae, Schizosaccharomyces pombe, Klayveromyces strains, Candida, or any yeast strain capable of expressing a RNAi agent that inhibits SH2B3, e.g., miRNA. Potentially suitable bacterial strains include Escherichia coli, Bacillus subtilis, Salmonella typhimurium, or any bacterial strain capable of expressing a RNAi agent that inhibits SH2B3, e.g., miRNA.

In another approach, genomic DNA encoding a RNAi agent that inhibits SH2B3, e.g., miRNA is isolated, the genomic DNA is expressed in a mammalian expression system, and RNA is purified and modified as necessary for administration to a patient. In some embodiments, a RNAi agent that inhibits SH2B3 which is a miRNA is in the form of a pre-miRNA, which can be modified as desired (i.e. for increased stability or cellular uptake).

Knowledge of DNA sequences of a RNAi agent that inhibits SH2B3, e.g., shRNA or miRNA allows for modification of cells to permit or increase expression of an endogenous miRNA. Cells can be modified (e.g., by homologous recombination) to provide increased miRNA expression by replacing, in whole or in part, the naturally occurring promoter with all or part of a heterologous promoter so that the cells express the RNAi agent that inhibits SH2B3, e.g., miRNA at higher levels. The heterologous promoter is inserted in such a manner that it is operatively linked to the desired a RNAi agent that inhibits SH2B3, e.g., miRNA encoding sequences. See, for example, PCT International Publication No. WO 94/12650 by Transkaryotic Therapies, Inc., PCT International Publication No. WO 92/20808 by Cell Genesys, Inc., and PCT International Publication No. WO 91/09955 by Applied Research Systems. Cells also may be; engineered to express an endogenous gene comprising a RNAi agent that inhibits SH2B3 under the control of inducible regulatory elements, in which case the regulatory sequences of the endogenous gene may be replaced by homologous recombination. Gene activation techniques are described in U.S. Pat. No. 5,272,071 to Chappel; U.S. Pat. No. 5,578,461 to Sherwin et al.; PCT/US92/09627 (WO93/09222) by Selden et al.; and PCT/US90/06436 (WO91/06667) by Skoultchi et al. The RNAi agent that inhibits SH2B3 may be prepared by culturing transformed host cells under culture conditions suitable to express the RNAi agent that inhibits SH2B3. The resulting expressed RNAi agent that inhibits SH2B3 may then be purified from such culture (i.e., from culture medium or cell extracts) using known purification processes, such as gel filtration and ion exchange chromatography. The purification of the RNAi agent that inhibits SH2B3, e.g., miR may also include an affinity column containing agents which will bind to the protein; one or more column steps over such affinity resins as concanavalin A-agarose, heparin-toyopearl™ or Cibacrom blue 3GA Sepharose™; one or more steps involving hydrophobic interaction chromatography using such resins as phenyl ether, butyl ether, or propyl ether; immunoaffnity chromatography, or complementary cDNA affinity chromatography.

The RNAi agent that inhibits SH2B3 can also be expressed as a product of transgenic animals, which are characterized by somatic or germ cells containing a nucleotide sequence encoding the RNAi agent that inhibits SH2B3. A vector containing DNA encoding RNAi agent that inhibits SH2B3 and appropriate regulatory elements can be inserted in the germ line of animals using homologous recombination (Capecchi, Science t 244:1288-1292 (1989)), such that they express the RNAi agent that inhibits SH2B3. Transgenic animals, preferably non-human mammals, are produced using methods as described in U.S. Pat. No 5,489,743 to Robinson, et al., and PCT Publication No. WO 94/28122 by Ontario Cancer Institute. In some embodiments, a RNAi agent that inhibits SH2B3 can be isolated from cells or tissue isolated from transgenic animals as discussed above.

In one approach, the RNAi agent that inhibits SH2B3 can be obtained synthetically, for example, by chemically synthesizing a nucleic acid by any method of synthesis known to the skilled artisan. The synthesized RNAi agent that inhibits SH2B3 can then be purified by any method known in the art. Methods for chemical synthesis of nucleic acids include, but are not limited to, in vitro chemical synthesis using phosphotriester, phosphate or phosphoramidite chemistry and solid phase techniques, or via deoxynucleoside H-phosphonate intermediates (see U.S. Pat. No. 5,705,629 to Bhongle).

In some circumstances, for example, where increased nuclease stability is desired, nucleic acids having nucleic acid analogs and/or modified internucleoside linkages may be preferred. Nucleic acids containing modified internucleoside linkages can also be synthesized using reagents and methods that are well known in the art. For example, methods of synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide (—CH2-S—CH2), diinethylene-sulfoxide (—CH2-SO—CH2), dimethylene-sulfone (—CH2-SO2-CH2), 2′-O-alkyl, and 2′-deoxy-2′-fluoro′ phosphorothioate internucleoside linkages are well known in the art (see Uhlmann et al., 1990, Chem. Rev. 90:543-584; Schneider et al., 1990, Tetrahedron Lett. 31:335 and references cited therein). U.S. Pat. Nos. 5,614,617 and 5,223,618 to Cook, et al., U.S. Pat. No. 5,714, 606 to Acevedo, et al, U.S. Pat. No. 5,378,825 to Cook, et al., U.S. Pat. Nos. 5,672,697 and 5,466, 786 to Buhr, et al., U.S. Pat. No. 5, 777,092 to Cook, et al., U.S. Pat. No. 5,602,240 to De Mesmacker, et al., U.S. Pat. No. 5,610,289 to Cook, et al. and U.S. Pat. No. 5,858,988 to Wang, also describe nucleic acid analogs for enhanced nuclease stability and cellular uptake.

In some embodiments, RNAi agent that inhibits SH2B3 can comprise locked nucleotides, e.g., as disclosed in U.S. Pat. No. 8,642,751, which is incorporated herein in its entirety by reference. Other chemical modifications of motifs for RNAi agent that inhibits SH2B3 as disclosed herein are disclosed in US application US 2012/0148664 and US 2014/0066491, which are incorporated herein in their entirety by reference. In alternative embodiments, RNAi agent that inhibits SH2B3 as disclosed herein can comprise base modified oligonucleotides, e.g., as disclosed in International Applications WO 2012061810 and WO 2012061810, which are incorporated herein in their entirety by reference.

### Oligonucleotide Formulations:

A formulated oligonucleotide composition can assume a variety of states. In some examples, the composition can be at least partially crystalline, uniformly crystalline, and/or anhydrous (e.g., less than 80, 50, 30, 20, or 10% water). In another example, the oligonucleotide is in an aqueous phase, e.g., in a solution that includes water.

The aqueous phase or the crystalline compositions can, e.g., be incorporated into a delivery vehicle, e.g., a liposome (particularly for the aqueous phase) or a particle (e.g., a micro particle as can be appropriate for a crystalline composition). Generally, the oligonucleotide composition is formulated in a manner that is compatible with the intended method of administration.

In particular embodiments, the composition is prepared by at least one of the following methods: spray drying, lyophilization, vacuum drying, evaporation, fluid bed drying, or a combination of these techniques; or sonication with a lipid, freeze-drying, condensation and other self-assembly.

An oligonucleotide preparation can be formulated in combination with another agent, e.g., another therapeutic agent or an agent that stabilizes the oligonucleotide, e.g., a protein that complex with oligonucleotide to form an oligonucleotide-protein complex. Still other agents include chelators, e.g., EDTA (e.g., to remove divalent cations such as Mg²⁺), salts, DNAse inhibitors, RNAse inhibitors (e.g., a broad specificity RNAse inhibitor such as RNAsin) and so forth.

In some embodiments, the oligonucleotide preparation includes at least a second therapeutic agent (e.g., an agent other than RNA or DNA). Exemplary therapeutic agents that can formulated with an oligonucleotide preparation include, but are not limited to, those found in Harrison's Principles of Internal Medicine, 17^{th} Edition, 2008, McGraw-Hill N.Y., N.Y.; Physicians Desk Reference, 63^{rd} Edition, 2008, Thomson Reuters, N.Y., N.Y.; Goodman & Gilman's The Pharmacological Basis of Therapeutics, 11^{th} Edition, 2005, McGraw-Hill N.Y., N.Y.; United States Pharmacopeia, The National Formulary, USP-32 NF-27, 2008, U.S. Pharmacopeia, Rockville, Md., the complete contents of all of which are incorporated herein by reference.

In some embodiments, the second therapeutic agent is an anti-hypertension agent or anti-hypertensive.

### Exemplary Oligonucleotide Formulations

Liposomes: The oligonucleotides of the invention (e.g., RNAi agent that inhibits SH2B3) can be formulated in liposomes. As used herein, a liposome is a structure having lipid-containing membranes enclosing an aqueous interior. Liposomes can have one or more lipid membranes. In some embodiments, liposomes have an average diameter of less than about 100 nm. More preferred embodiments provide liposomes having an average diameter from about 30-70 nm and most preferably about 40-60 nm. Oligolamellar large vesicles and multilamellar vesicles have multiple, usually concentric, membrane layers and are typically larger than 100 nm. Liposomes with several nonconcentric membranes, i.e., several smaller vesicles contained within a larger vesicle, are termed multivesicular vesicles.

Liposomes can further comprise one or more additional lipids and/or other components such as sterols, e.g., cholesterol. Additional lipids can be included in the liposome compositions for a variety of purposes, such as to prevent lipid oxidation, to stabilize the bilayer, to reduce aggregation during formation or to attach ligands onto the liposome surface. Any of a number of additional lipids and/or other components can be present, including amphipathic, neutral, cationic, anionic lipids, and programmable fusion lipids. Such lipids and/or components can be used alone or in combination. One or more components of the liposome can comprise a ligand, e.g., a targeting ligand.

Liposome compositions can be prepared by a variety of methods that are known in the art. See e.g., U.S. Pat. Nos. 4,235,871; 4,737,323; 4,897,355 and 5,171,678; published International Applications WO 96/14057 and WO 96/37194; Felgner, P. L. et al., Proc. Natl. Acad. Sci., USA (1987) 8:7413-7417, Bangham, et al. M. Mol. Biol. (1965) 23:238, Olson, et al. Biochim. Biophys. Acta (1979) 557:9, Szoka, et al. Proc. Natl. Acad. Sci. (1978) 75: 4194, Mayhew, et al. Biochim. Biophys. Acta (1984) 775:169, Kim, et al. Biochim. Biophys. Acta (1983) 728:339, and Fukunaga, et al. Endocrinol. (1984) 115:757.

Micelles and other Membranous Formulations: In some embodiments, the oligonucleotides (e.g., a RNAi agent that inhibits SH2B3) as disclosed herein can be prepared and formulated as micelles. As used herein, “micelles” are a particular type of molecular assembly in which amphipathic molecules are arranged in a spherical structure such that all hydrophobic portions on the molecules are directed inward, leaving the hydrophilic portions in contact with the surrounding aqueous phase. The converse arrangement exists if the environment is hydrophobic.

In some embodiments, the formulations comprises micelles formed from an oligonucleotide of the invention and at least one amphiphilic carrier, in which the micelles have an average diameter of less than about 100 nm, preferably. More preferred embodiments provide micelles having an average diameter less than about 50 nm, and even more preferred embodiments provide micelles having an average diameter less than about 30 nm, or even less than about 20 nm.

Micelle formulations can be prepared by mixing an aqueous solution of the oligonucleotide composition, an alkali metal C₈ to C₂₂ alkyl sulphate, and an amphiphilic carrier. The amphiphilic carrier can be added at the same time or after addition of the alkali metal alkyl sulphate. Micelles will form with substantially any kind of mixing of the ingredients but vigorous mixing in order to provide smaller size micelles.

Emulsions: In some embodiments, the oligonucleotides (e.g., RNAi agent that inhibits SH2B3) as disclosed herein can be prepared and formulated as emulsions. As used herein, “emulsion” is a heterogenous system of one liquid dispersed in another in the form of droplets. Emulsions are often biphasic systems comprising two immiscible liquid phases intimately mixed and dispersed with each other. Either of the phases of the emulsion can be a semisolid or a solid, as is the case of emulsion-style ointment bases and creams. The oligonucleotide can be present as a solution in either the aqueous phase, oily phase or itself as a separate phase.

In some embodiments, the compositions are formulated as microemulsions. As used herein, “microemulsion” refers to a system of water, oil and amphiphile which is a single optically isotropic and thermodynamically stable liquid solution. Microemuslions also include thermodynamically stable, isotropically clear dispersions of two immiscible liquids that are stabilized by interfacial films of surface-active molecules.

The application of emulsion formulations via dermatological, oral and parenteral routes and methods for their manufacture have been reviewed in the literature, for example see Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., volume 1, p. 245; and Block, in Pharmaceutical Dosage Forms, Lieberman, Rieger and Banker (Eds.), 1988, Marcel Dekker, Inc., New York, N.Y., volume 1, p. 335, contents of which are herein incorporated by reference in their entirety.

Lipid Particles: In some embodiments, the oligonucleotides (e.g., a RNAi agent that inhibits SH2B3) as disclosed herein can be prepared and formulated as lipid particles, e.g., formulated lipid particles (FLiPs) comprising (a) an oligonucleotide of the invention, where said oligonucleotide has been conjugated to a lipophile and (b) at least one lipid component, for example an emulsion, liposome, isolated lipoprotein, reconstituted lipoprotein or phospholipid, to which the conjugated oligonucleotide has been aggregated, admixed or associated. The stoichiometry of oligonucleotide to the lipid component can be 1:1. Alternatively the stoichiometry can be 1:many, many:1 or many:many, where many is two or more.

The FLiP can comprise triacylglycerols, phospholipids, glycerol and one or several lipid-binding proteins aggregated, admixed or associated via a lipophilic linker molecule with an oligonucleotide. Surprisingly, it has been found that due to said one or several lipid-binding proteins in combination with the above mentioned lipids, the FLiPs show affinity to liver, gut, kidney, steroidogenic organs, heart, lung and/or muscle tissue. These FLiPs can therefore serve as carrier for oligonucleotides to these tissues. For example, lipid-conjugated oligonucleotides, e.g., cholesterol-conjugated oligonucleotides, bind to HDL and LDL lipoprotein particles which mediate cellular uptake upon binding to their respective receptors thus directing oligonucleotide delivery into liver, gut, kidney and steroidogenic organs, see Wolfrum et al. Nature Biotech. (2007), 25:1145-1157.

The FLiP can be a lipid particle comprising 15-25% triacylglycerol, about 0.5-2% phospholipids and 1-3% glycerol, and one or several lipid-binding proteins. FLiPs can be a lipid particle having about 15-25% triacylglycerol, about 1-2% phospholipids, about 2-3% glycerol, and one or several lipid-binding proteins. In some embodiments, the lipid particle comprises about 20% triacylglycerol, about 1.2% phospholipids and about 2.25% glycerol, and one or several lipid-binding proteins.

Another suitable lipid component for FLiPs is lipoproteins, for example isolated lipoproteins or more preferably reconstituted lipoprotieins. Exemplary lipoproteins include chylomicrons, VLDL (Very Low Density Lipoproteins), IDL (Intermediate Density Lipoproteins), LDL (Low Density Lipoproteins) and HDL (High Density Lipoproteins). Methods of producing reconstituted lipoproteins are known in the art, for example see A. Jones, Experimental Lung Res. 6, 255-270 (1984), U.S. Pat. Nos. 4,643,988 and 5,128,318, PCT publication WO87/02062, Canadian Pat. No. 2,138,925. Other methods of producing reconstituted lipoproteins, especially for apolipoproteins A-I, A-II, A-IV, apoC and apoE have been described in A. Jonas, Methods in Enzymology 128, 553-582 (1986) and G. Franceschini et al. J. Biol. Chem., 260(30), 16321-25 (1985).

One preferred lipid component for FLiP is Intralipid. Intralipid® is a brand name for the first safe fat emulsion for human use. Intralipid® 20% (a 20% intravenous fat emulsion) is made up of 20% soybean oil, 1.2% egg yolk phospholipids, 2.25% glycerin, and water for injection. It is further within the present invention that other suitable oils, such as saflower oil, can serve to produce the lipid component of the FLiP.

FLiP can range in size from about 20-50 nm or about 30-50 nm, e.g., about 35 nm or about 40 nm. In some embodiments, the FLiP has a particle size of at least about 100 nm. FLiPs can alternatively be between about 100-150 nm, e.g., about 110 nm, about 120 nm, about 130 nm, or about 140 nm, whether characterized as liposome- or emulsion-based. Multiple FLiPs can also be aggregated and delivered together, therefore the size can be larger than 100 nm.

The process for making the lipid particles comprises the steps of: (a) mixing a lipid components with one or several lipophile (e.g. cholesterol) conjugated oligonucleotides that can be chemically modified; and (b) fractionating this mixture. In some embodiments, the process comprises the additional step of selecting the fraction with particle size of 30-50 nm, preferably of about 40 nm in size.

Some exemplary lipid particle formulations amenable to the invention are described in U.S. patent application Ser. No.12/412,206, filed Mar. 26, 2009, contents of which are herein incorporated by reference in their entirety.

Yeast cell wall particles: In some embodiments, the oligonucleotides (e.g., a RNAi agent that inhibits SH2B3) as disclosed herein are formulated in yeast cell wall particles (“YCWP”). A yeast cell wall particle comprises an extracted yeast cell wall exterior and a core, the core comprising a payload (e.g., oligonucleotides). Exterior of the particle comprises yeast glucans (e.g. beta glucans, beta-1,3-glucans, beta-1,6-glucans), yeast mannans, or combinations thereof. Yeast cell wall particles are typically spherical particles about 1-4 μm in diameter.

Preparation of yeast cell wall particles is known in the art, and is described, for example in U.S. Pat. Nos. 4,992,540; 5,082,936; 5,028,703; 5,032,401; 5,322,841; 5,401,727; 5,504,079; 5,607,677; 5,741,495; 5,830,463; 5,968,811; 6,444,448; and 6,476,003, U.S. Pat. App. Pub. Nos. 2003/0216346 and 2004/0014715, and Int. App. Pub. No. WO 2002/12348, contents of which are herein incorporated by reference in their entirety. Applications of yeast cell like particles for drug delivery are described, for example in U.S. Pat. Nos. 5,032,401; 5,607,677; 5,741,495; and 5,830,463, and U.S. Pat. Pub Nos. 2005/0281781 and 2008/0044438, contents of which are herein incorporated by reference in their entirety. U.S. Pat. App. Pub. No. 2009/0226528, contents of which are herein incorporated by reference, describes formulation of nucleic acids with yeast cell wall particles for delivery of oligonucleotide to cells.

Additional exemplary formulations for oligonucleotides are described in U.S. Pat. Nos. 4,897,355; 4,394,448; 4,235,871; 4,231,877; 4,224,179; 4,753,788; 4,673,567; 4,247,411; 4,814,270; 5,567,434; 5,552,157; 5,565,213; 5,738,868; 5,795,587; 5,922,859; and 6,077,663, Int. App. Nos. PCT/US07/079203, filed Sep. 21, 2007; PCT/US07/080331, filed Oct. 3, 2007; U.S. patent application Ser. No. 12/123,922, filed May 28, 2008; U.S. Pat. Pub. Nos. 2006/0240093 and 2007/0135372 and U.S. Provisional App. Nos. 61/018,616, filed Jan. 2, 2008; 61/039,748, filed Mar. 26; 2008; 61/045,228, filed Apr. 15, 2008; 61/047,087, filed Apr. 22, 2008; 61/051,528, filed May 21, 2008; and 61/113,179 (filed Nov. 10, 2008), contents of which are herein incorporated by reference in their entirety. Behr (1994) Bioconjugate Chem. 5:382-389, and Lewis et al. (1996) PNAS 93:3176-3181), also describe formulations for oligonucleotides that are amenable to the invention, contents of which are herein incorporated by reference in their entirety.

Small Molecule Inhibitors of SH2B3

SH2B3 protein has both an SH2 domain and PH domain, either of which or both can be a target for development of peptide and small molecule inhibitors. See D. Kraskouskaya, et al., Chem Soc Rev. 2013 Apr. 21; 42(8):3337-70. In some embodiments, the antagonist of SH2B3 specifically binds to the SH2 domain of SH2B3 protein. In some embodiments, the antagonist of SH2B3 specifically binds to the PH domain of SH2B3 protein. In some embodiments, the antagonist of SH2B3 specifically binds to both the SH2 and PH domains of SH2B3 protein.

In some embodiments, the antagonist of SH2B3 is a small molecule. As used herein, the term “small molecule” refers to a natural or synthetic molecule having a molecular mass of less than about 5 kD, organic or inorganic compounds having a molecular mass of less than about 5 kD, less than about 2 kD, or less than about 1 kD.

In some embodiments, the antagonist of SH2B3 can have an IC50 of less than 50 μM, e.g., the antagonist of SH2B3 can have an IC50 of from about 50 μM to about 5 nM, or less than 5 nM. For example, in some embodiments, an antagonist of SH2B3 has an IC50 of from about 50 μM to about 25 μM, from about 25 μM to about 10 μM, from about 10 μM to about 5 μM, from about 5 μM to about 1 μM, from about 1 μM to about 500 nM, from about 500 nM to about 400 nM, from about 400 nM to about 300 nM, from about 300 nM to about 250 nM, from about 250 nM to about 200 nM, from about 200 nM to about 150 nM, from about 150 nM to about 100 nM, from about 100 nM to about 50 nM, from about 50 nM to about 30 nM, from about 30 nM to about 25 nM, from about 25 nM to about 20 nM, from about 20 nM to about 15 nM, from about 15 nM to about 10 nM, from about 10 nM to about 5 nM, or less than about 5 nM.

In some embodiments, the antagonist of SH2B3 can be an anti-SH2B3 antibody molecule or an antigen-binding fragment thereof. Suitable antibodies include, but are not limited to, polyclonal, monoclonal, chimeric, humanized, recombinant, single chain, F_{ab}, F_{ab′}, F_{sc}, Rᵥ, and F_{(ab′)2} fragments. In some embodiments, neutralizing antibodies can be used as anti-SH2B3 antibodies. Antibodies are readily raised in animals such as rabbits or mice by immunization with the antigen Immunized mice are particularly useful for providing sources of B cells for the manufacture of hybridomas, which in turn are cultured to produce large quantities of monoclonal antibodies. In general, an antibody molecule obtained from humans can be classified in one of the immunoglobulin classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

Antibodies provide high binding avidity and unique specificity to a wide range of target antigens and haptens. Monoclonal antibodies useful in the practice of the methods disclosed herein include whole antibody and fragments thereof and are generated in accordance with conventional techniques, such as hybridoma synthesis, recombinant DNA techniques and protein synthesis.

The SH2B3 polypeptide, or a portion or fragment thereof, can serve as an antigen, and additionally can be used as an immunogen to generate antibodies that immunospecifically bind the antigen, using standard techniques for polyclonal and monoclonal antibody preparation. Preferably, the antigenic peptide comprises at least 10 amino acid residues, or at least 15 amino acid residues, or at least 20 amino acid residues, or at least 30 amino acid residues.

Useful monoclonal antibodies and fragments can be derived from any species (including humans) or can be formed as chimeric proteins which employ sequences from more than one species. Human monoclonal antibodies or “humanized” murine antibody can also be used in accordance with the present invention. For example, murine monoclonal antibody can be “humanized” by genetically recombining the nucleotide sequence encoding the murine Fv region (i.e., containing the antigen binding sites) or the complementarily determining regions thereof with the nucleotide sequence encoding a human constant domain region and an Fc region. Humanized targeting moieties are recognized to decrease the immunoreactivity of the antibody or polypeptide in the host recipient, permitting an increase in the half-life and a reduction in the possibility of adverse immune reactions in a manner similar to that disclosed in European Patent Application No. 0,411,893 A2. The murine monoclonal antibodies should preferably be employed in humanized form. Antigen binding activity is determined by the sequences and conformation of the amino acids of the six complementarily determining regions (CDRs) that are located (three each) on the light and heavy chains of the variable portion (Fv) of the antibody. The 25-kDa single-chain Fv (scFv) molecule, composed of a variable region (VL) of the light chain and a variable region (VH) of the heavy chain joined via a short peptide spacer sequence, is one option for minimizing the size of an antibody agent. ScFvs provide additional options for preparing and screening a large number of different antibody fragments to identify those that specifically bind. Techniques have been developed to display scFv molecules on the surface of filamentous phage that contain the gene for the scFv. scFv molecules with a broad range orantigenic-specificities can be present in a single large pool of scFv-phage library.

Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. Preferably, both regions and the combination have low immunogenicity as routinely determined.

Anti-SH2B3 antibodies are commercially available through vendors such as Thermo Scientific, Sigma Aldrich, Atlas Antibodies, and R&D Systems.

Methods of culturing stem cells and/or progenitor cells are well known in the art. In general, cells as described herein can be cultured in culture medium that is available to and well-known in the art. Such media include, but are not limited to, Dulbecco's Modified Eagle's, F-12K®, Eagle's Minimum Essential Medium® (DMEM), DMEM F12 Medium®, and serum-free®, RPMI-1640 Medium®, Iscove's Modified Dulbecco's Medium® (IMDM). Many media for culture and expansion of stem cells and/or progenitor cells are also available as low-glucose formulations, with or without sodium pyruvate.

Also contemplated herein is supplementation of cell culture medium with mammalian sera. Sera often contain cellular factors and components that are necessary for viability and expansion. Examples of sera include fetal bovine serum (FBS), bovine serum (BS), calf serum (CS), fetal calf serum (FCS), newborn calf serum (NCS), goat serum (GS), horse serum (HS), human serum (e.g., human AB serum), chicken serum, porcine serum, sheep serum, rabbit serum, serum replacements and bovine embryonic fluid. It is understood that sera can be heat-inactivated at 55-65° C. if deemed necessary to inactivate components of the complement cascade. Plasma such as human AB plasma can also be used.

Growth factors can be added to the cell culture medium. Hematopoietic growth factors include, but are not limited to, any or all Interleukins (IL-1 to IL-16), interferons (IFN-alpha, beta and gamma), erythropoietin (EPO), stem cell factor (SCF), insulin like growth factors, fibroblast growth factors, platelet-derived growth factor, tumor growth factor beta, tumor necrosis factor alpha, granulocyte colony stimulating factor (G-CSF), granulocyte-macrophage colony stimulating factor (GM-CSF), macrophage colony stimulating factor (M-CSF), fins-like tyrosine kinase-3 ligand (Flt3-ligand), as well as EGF (epidermal growth factor), VEGF (vascular endothelial growth factor), LIF (leukemia inhibiting factor). Thrombopoletin (TPO) or MGDF (mast growth derived factor) may also be used. Many of these growth factors are commercially available. Most commonly used mixture of growth factors includes G-CSF, GM-CSF, SCF, IL-1, IL-3 and IL-6. Most of the growth factors used are produced by recombinant DNA techniques are purified to various degrees. Some growth factors are purified from culture media of tumor cell lines by standard biochemical techniques. A widely used growth factor is PIXY 321 which is produced by recombinant technology and exhibits both, GM-CSF and IL-3 activity.

The amount of growth factors used in the cultures depends on the activity of the factor preparation and on the combination of growth factors used. Typically, concentrations range from 0.5 to 500 ng/mL. The optimum concentration of each growth factor has to be determined for individual culture conditions since some growth factors act synergistically with other growth factors.

Additional supplements also can be used advantageously to supply the cells with the necessary trace elements for optimal growth and expansion. Such supplements include, for example, insulin, transferrin, heparin, sodium selenium and combinations thereof. These components can be included in a salt solution including, but not limited to, (HBSS), Earle's Salt®, Hanks' Balanced Salt Solution, antioxidant supplements, MCDB-201© Solution saline (PBS), ascorbic acid and ascorbic acid-2-phosphate, as well as additional amino acids. Many cell culture media already contain amino acids, however, some require supplementation prior to culturing cells. Such amino acids include, but are not limited to, L-alanine, L-arginine, L-aspartic acid, L-asparagine, L-cysteine, L-cystine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine. It is well within the skill of one in the art to determine the proper concentrations of these supplements.

Hormones also can be advantageously used in the cell cultures described herein and include, but are not limited to, D-aldosterone, diethylstilbestrol (DES), dexamethasone, beta-estradiol, hydrocortisone, insulin, prolactin, progesterone, somatostatin/human growth hormone (HGH), thyrotropin, thyroxine and L-thyronine.

Lipids and lipid carriers also can be used to supplement cell culture media, depending on the type of cell and the fate of the differentiated cell. Such lipids and carriers can include, but are not limited to, cholesterol, linoleic acid conjugated to albumin, cyclodextrin, linoleic acid and oleic acid conjugated to albumin, unconjugated linoleic acid, among others.

Also contemplated for the methods described herein is the use of feeder cell layers. Feeder cells are used to support the growth of fastidious cultured cells, such as stem cells. Feeder cells are normal cells that have been γ-irradiated to suppress cell division yet permit active metabolism. In culture, the feeder layer serves as a basal inactivated layer for other cells and supplies cellular factors without further growth or division of their own (Lim, J. W. and Bodnar, A., 2002). Examples of typical feeder layer cells include human diploid lung cells, mouse embryonic fibroblasts and Swiss mouse embryonic fibroblasts, but can be any post-mitotic cell that is capable of supplying cellular components and factors that are advantageous in allowing optimal growth, viability and expansion of stem cells and/or progenitor cells. In many cases, feeder cell layers are not necessary to keep the stem cells and/or progenitor cells in an undifferentiated, proliferative state, as leukemia inhibitory factor (LIF) has anti-differentiation properties. Therefore, supplementation with LIF can be used to maintain cells in an undifferentiated state.

Cells can be cultured in low-serum or serum-free culture medium. Serum-free medium used to culture cells is described in, for example, U.S. Pat. No. 7,015,037. Many cells have been grown in serum-free or low-serum medium. For example, the medium can be supplemented with one or more growth factors. Commonly used growth factors include, but are not limited to, bone morphogenic protein, basic fibroblast growth factor, platelet-derived growth factor and epidermal growth factor, Stem cell factor, thrombopoietin, Flt3Ligand and Γ-3. See, for example, U.S. Pat. Nos. 7,169,610; 7,109,032; 7,037,721; 6,617,161; 6,617,159; 6,372,210; 6,224,860; 6,037,174; 5,908,782; 5,766,951; 5,397,706; and 4,657,866; all incorporated by reference herein for the teachings of growing cells in serum-free medium.

Cells in culture can be maintained either in suspension or attached to a solid support, such as extracellular matrix components. Stem cells and/or progenitor cells often require additional factors that encourage their attachment to a solid support, such as type I and type II collagen, chondroitin sulfate, fibronectin, “superfibronectin” and fibronectin-like polymers, gelatin, poly-D and poly-L-lysine, thrombospondin and vitronectin. Stem cells and/or progenitor cells can also be cultured in low attachment flasks such as but not limited to Corning Low attachment plates.

In some embodiments, the stem cells and/or progenitor cells can be cultured in a multi-phase (e.g., three-phase) culture system, in which the composition of grown factors varies during the course of cell growth and differentiation. For example, IL-3, SCF, and EPO can be used at the early stage, SCF and EPO used at the intermediate stage, and EPO used at the last stage of cell growth and differentiation.

In some embodiments, the stem cells and/or progenitor cells are cultured in the presence of at least 1.0 unit/ml EPO, at least 1.2 units/ml EPO, at least 1.4 units/ml EPO, at least 1.6 units/ml EPO, at least 1.8 units/ml EPO, at least 2.0 units/ml EPO, at least 2.2 units/ml EPO, at least 2.4 units/ml EPO, at least 2.6 units/ml EPO, at least 2.8 units/ml EPO, at least 3.0 units/ml EPO, at least 3.2 units/ml EPO, at least 3.4 units/ml EPO, at least 3.6 units/ml EPO, at least 3.8 units/ml EPO, or at least 4.0 units/ml EPO.

In some embodiments, the stem cells and/or progenitor cells are cultured in the presence of at least 1 ng/ml IL-3, at least 2 ng/ml IL-3, at least 3 ng/ml IL-3, at least 4 ng/ml IL-3, at least 5 ng/ml IL-3, at least 6 ng/ml IL-3, at least 7 ng/ml IL-3, at least 8 ng/ml IL-3, at least 9 ng/ml IL-3, at least 10 ng/ml IL-3, at least 11 ng/ml IL-3, at least 12 ng/ml IL-3, at least 13 ng/ml IL-3, at least 14 ng/ml IL-3, at least 15 ng/ml IL-3, at least 16 ng/ml IL-3, at least 17 ng/ml IL-3, at least 18 ng/ml IL-3, at least 19 ng/ml IL-3, at least 20 ng/ml IL-3, at least 21 ng/ml IL-3, at least 22 ng/ml IL-3, at least 23 ng/ml IL-3, at least 24 ng/ml IL-3,or at least 25 ng/ml IL-3.

In some embodiments, the stem cells and/or progenitor cells are cultured in the presence of at least 1 ng/ml SCF, at least 2 ng/ml SCF, at least 3 ng/ml SCF, at least 4 ng/ml SCF, at least 5 ng/ml SCF, at least 6 ng/ml SCF, at least 7 ng/ml SCF, at least 8 ng/ml SCF, at least 9 ng/ml SCF, at least 10 ng/ml SCF, at least 15 ng/ml SCF, at least 20 ng/ml SCF, at least 25 ng/ml SCF, at least 30 ng/ml SCF, at least 35 ng/ml SCF, at least 40 ng/ml SCF, at least 45 ng/ml SCF, at least 50 ng/ml SCF, at least 55 ng/ml SCF, at least 60 ng/ml SCF, at least 65 ng/ml SCF, at least 70 ng/ml SCF, at least 75 ng/ml SCF, at least 80 ng/ml SCF, at least 85 ng/ml SCF, at least 90 ng/ml SCF, at least 95 ng/ml SCF, at least 100 ng/ml SCF, at least 150 ng/ml SCF, at least 200 ng/ml SCF, at least 250 ng/ml SCF, at least 300 ng/ml SCF, at least 350 ng/ml SCF, at least 400 ng/ml SCF, at least 450 ng/ml SCF, or at least 500 ng/ml SCF.

In some embodiments, HSCs are cultured in the presence of IL-3, SCF, EPO, human AB plasma, human AB serum, transferrin, heparin, and insulin.

Cell culture media for stem cells for the production of RBCs are disclosed, for example in US20140255369 and US8206979, the contents of each of which are incorporated herein by reference in their entirety.

### Compositions Comprising RBC

In some embodiments, the methods described herein can increase the amount of RBCs by at least 10%, at least 20%, at least 30%, at least 40%, at least 50%, at least 60%, at least 70%, at least 80%, at least 90%, at least 100%, at least 200%, at least 300%, at least 400%, or at least 500%, as compared to a method using the same population of stem cells and/or progenitor cells without any SH2B3 inhibition.

In some embodiments, the methods described herein can increase the quality of RBCs by at least 10%, at least 20%, at least 30%, at least 40%, at least 50%, at least 60%, at least 70%, at least 80%, at least 90%, at least 100%, at least 200%, at least 300%, at least 400%, or at least 500%, as compared to a method using the same population of stem cells and/or progenitor cells without any SH2B3 inhibition. The quality of RBCs is considered to be increased when the percentage of enucleated RBCs is increased, the frequency of RBCs having appropriate markers of maturation is increased, hemoglobinization is improved, or combinations thereof. Markers of maturation include surface expression of CD235a (glycophorin A), rhesus antigen, CD44, and a variety of other blood types expressed on mature red blood cells.

In some embodiments, the methods described herein can increase the rate of RBCs towards maturation by at least 10%, at least 20%, at least 30%, at least 40%, at least 50%, at least 60%, at least 70%, at least 80%, at least 90%, at least 100%, at least 200%, at least 300%, at least 400%, or at least 500%, as compared to a method using the same population of stem cells and/or progenitor cells without any SH2B3 inhibition.

In some embodiments, the method as disclosed herein comprises contacting the HSPCs with a SH2B3 antagonist for a pre-determined period of time. In some embodiments, the method as disclosed herein comprises contacting the HSPCs with a SH2B3 antagonist for the pre-determined times of at least about 2 hrs, or at least about 4 hours, or at least about 8 hours, or at least about 10 hours, or at least about 12 hours, or between 12-24 hours, or between 24-32 hours, or between 32-48 hours, or between 1-2 days, or between 3-4 days or longer than 4 days.

In some embodiments, the contacting occurs for a pre-selected period of time prior to, or during phase 1 (day 1-7) differentiation, i.e., in the presence of a culure media comprising heparin, holo-transferrin, erythropoietin (Epo), stem cell factor (SCF) and IL-3), and at least one SH2B3 antagonist. In some embodiments, the contacting occurs prior to, or during phase 1 (day 1-7) differentiation, i.e., in the presence of a culure media comprising heparin, holo-transferrin, erythropoietin (Epo), stem cell factor (SCF) and IL-3), and at least one SH2B3 antagonist.

In some embodiments, the SH2B3 antagonist contacts the HSPCs for a pre-selected period of time prior to, or during phase 2 (day 7-day 12) differentiation, i.e., in the presence of a culture media comprising heparin, holo-transferrin, erythropoietin (Epo), and stem cell factor (SCF)), and at least one SH2B3. In some embodiments, the SH2B3 antagonist contacts the HSPCs for a pre-selected period of time prior to, or during phase 3 (day 12-day 18) differentiation, i.e., in the presence of a culure media comprising heparin, holo-transferrin and erythropoietin (Epo)), and at least one SH2B3 antagonist.

In some embodiments, the population of mature RBCs is produced in 7 to 21 days. In some embodiments, the population of mature RBCs is produced in 7 to 18 days. In some embodiments, the population of mature RBCs is produced in 7 to 16 days. In some embodiments, the population of mature RBCs is produced in 7 to 14 days. In some embodiments, the population of mature RBCs is produced in 7 to 12 days. In some embodiments, the population of mature RBCs is produced in less than 7 days.

In some embodiments, the methods and compositions as disclosed herein comprise RBC generated by the methods as disclosed herein. In some embodiments, the present invention encompasses an admixture of RBC in combination with the pluripotent stem cells or progenitor cells (i.e., HSPCs) from which they were derived, and in some embodiments, the admixture comprises less than about 25%, or less than about 20%, or less than about 15%, or less than about 13%, or less than about 12%, or less than about 11%, or less than about 10%, or less than about 9%, or less than about 8%, or less than about 7%, or less than about 6%, or less than about 5%, or less than about 4%, or less than about 3%, or less than about 2%, or less than about 1% of stem cells and/or progenitor cells in the total cell population of the admixture.

In some embodiments, RBC are the majority cell type in the admixture population of cells. For instance, the methods produce a cell population described herein, or an admixture described herein comprises at least about 99%, or at least about 98%, or at least about 97%, or at least about 96%, or at least about 95%, or at least about 94%, or at least about 93%, or at least about 92%, or at least about 91%, or at least about 90%, or at least about 89%, or at least about 87%, or at least about 86%, or at least about 85%, or at least about 84%, or at least about 83%, or at least about 82%, or at least about 81%, or at least about 80%, or at least about 79%, or at least about 78%, or at least about 77%, or at least about 76%, or at least about 75%, or at least about 74%, or at least about 73%, or at least about 72%, or at least about 71%, or at least about 70%, or at least about 69%, or at least about 68%, or at least about 67%, or at least about 65%, or at least about 64%, or at least about 63%, or at least about 62%, or at least about 61%, or at least about 60%, or at least about 59%, or at least about 58%, or at least about 57%, or at least about 56%, or at least about 55%, or at least about 50% RBC. In some embodiments, the percentage of RBC in an admixture or culture, or cell population generated using the methods as disclosed herein is calculated without regard to feeder cells or other cells used to maintain the culture.

RBCs produced using the methods and compositions as described herein can be stored in a blood bank. In some embodiments, the RBC are present in a cryopreservation media or storage medium. In some embodiments, the RBC are stored at +4° C. or colder, e.g., −20° C. in an appropriate storage media. The blood bank can provide a large pool of available RBCs, that can be utilized in a variety of therapeutic, as well as research, applications. The stored RBCs can serve, for example, as a source of RBCs for use in the future when health reasons require blood transfusion, e.g., when the subject is to undergo a surgery and/or is in need of a blood transfusion at a later date (e.g., undergoing a bone marrow transplant, etc.). In some embodiments, the RBCs can be banked for sporting purposes, e.g., for blood transfusion prior to a sporting event. The stored RBCs can also serve as a source of cells for autologous use, for example, for future treatments of the donor. In some embodiments, the stored RBCs can also serve as a source of cells for future treatments of a relative of the donor. The stored RBCs can also serve as a source of cells for clinical use by other individuals with matched blood type upon authorization from the donor. Blood type compatibility is shown in Table 1. Also, the methods and compositions described herein can be used to produce RBCs for rare blood types, e.g., Duffy-negative blood. The methods and compositions described herein can be used to produce RBCs for a subject who changes blood type during his/her lifetime, e.g., a subject who is type A and receives type O bone marrow transplant.

Storage conditions for RBCs are well known in the art. Routine blood storage is 42 days or 6 weeks for stored packed RBCs. Methods for prolonged storage of RBC are well known in the art, and are described in U.S. Pat. No. 4,585,735, 4,675,185, 4,943,287, 5,789,151, 6,150,085, 8,071,282, 8,968,992, US patent Applications 2012/0329036, 2001/0049089, 2014/0091047, 2015/0190309, 2014/0086892, which are incorporated herein in their entireties by reference.

Another aspect of the present invention relates to a cell cuture media comprising RBCs produced using the methods as disclosed herein and at least one SH2B3 inhibitor as disclosed herein. In some embodiments, a cell cuture media comprising RBCs and at least one SH2B3 antagonist. In some embodiments, the SH2B3 is a siRNA agent. In some embodiments, the composition comprises a culture media comprising media for phase 1 (day 1-7) differentitaion (comprising heparin, holo-transferrin, erythropoietin (Epo), stem cell factor (SCF) and IL-3), and at least one SH2B3 antagonist. In some embodiments, the composition comprises a culture media comprising media for phase 2 (day 7-day 12) differentiation (comprising heparin, holo-transferrin, erythropoietin (Epo), and stem cell factor (SCF)), and at least one SH2B3. In some embodiments, the composition comprises a culture media comprising media for phase 3 (day 12-day 18) differentiation (comprising heparin, holo-transferrin and erythropoietin (Epo)), and at least one SH2B3 antagonist.

### RBC Collection

RBCs can be isolated using known methods in the art. For example, RBCs can be isolated by leukocyte filtration or flow cytometric sorting. In some embodiments, the RBCs can be collected based on positive selection for the CD235a marker and negative selection for the CD71 marker.

In some embodiments, RBCs are isolated using leukocyte filtration. In leukocyte filtration, almost all nucleated cells are removed by the use of a filter, so a substantially pure population of RBCs can be obtained. Leukocyte filtration is a useful method for large scale RBC isolation.

### Therapeutic Uses

RBCs produced according to any of the methods described herein can be used in a variety of therapeutic applications. The use of RBC transfusions for patients in need of such treatment for a variety of reasons and disorders is well known in the art, and approaches are standard medical practice. The RBCs described herein and/or produced according to the methods of the present disclosure can be used to treat patients using the same approaches and conditions currently used for blood transfusions.

In certain embodiments, the present disclosure relates to methods of treatment, prevention, or diagnosis of a disease or disorder characterized by a deficiency of red blood cells by administering a population of red blood cells of the present disclosure, or prepared according to any of the methods of the present disclosure, to a subject having a disorder characterized by a deficiency of red blood cells.

As used herein, a “deficiency of red blood cells,” refers to a subject that has an amount of red blood cells that is from about 20% to about 900% lower than the amount of red blood cells in a subject having a normal amount of red blood cells; or has an amount of red blood cells that is from about 10 times to about 1,000 times lower than the amount of red blood cells in a subject having a normal amount of red blood cells.

Disorders characterized by a deficiency of red blood cells may include, without limitation, anemia (e.g., congenital anemia, aplastic anemia, pernicious anemia, iron deficiency anemia, sickle cell anemia, spherocytosis, hemolytic anemia, Aceruloplasminemia, Adenosine deaminase increased activity, Adenylate kinase deficiency, Aldolase deficiency, Alpha-thalassaemia—trait or carrier, Atransferrinemia, Autosomal dominant sideroblastic anemia, Autosomal recessive sideroblastic anemia, Beta-thalassaemia—trait or carrier, Beta-thalassaemia major (and intermedia), CDA with thrombocytopenia (GATA I mutation), Compound heterozygous sickling disorders, Congenital acanthocytosis, Congenital dyserythropoietic anaemia type I, Congenital dyserythropoietic anaemia type II, Congenital dyserythropoietic anaemia type III, Delta Beta-thalassaemia, Diamond-Blackfan-Anemia, DMT1-deficiency anaemia, Familial hypoplastic anaemia, Fanconi anaemia, Gamma-glutamyl-cysteine synthetase deficiency, GLRX5-related Sideroblastic anaemia, Glucose phosphate isomerase deficiency, Glucose-6-phosphate dehydrogenase deficiency, Glutathione reductase deficiency, Glutathione synthetase deficiency, Haemoglobin C disease, Haemoglobin D disease, Haemoglobin E disease, Haemoglobin H disease, Haemoglobin Lepore, Haemoglobin M with anaemia, Hereditary Elliptocytosis, Hereditary persistance of fetal haemoglobin, Hereditary Spherocytosis, Hereditary Stomatocytosis, Hexokinase deficiency, Hydrops fetalis, Imerslund-Grasbeck-Syndrome, Iron-refractory iron deficiency anemia, Kearns-Sayre syndrome, Lecithin cholesterol acyltransferase deficiency, Mitochondrial myopathy sideroblastic anemia, thalassaemias, congenitale dyserythropoetic anemia, Pancytopenia with malformations, Paroxysmal nocturnal hemoglobinuria, Pearson's Syndrome, Phosphofructokinase deficiency, Phosphoglycerate kinase deficiency, Pyrimidine 5 nucleotidase deficiency, Pyruvate kinase deficiency, Sickle cell anemia, Sickle cell trait, Sideroblastic anemia associated with ataxia, SLC25A38-related Sideroblastic anemia, Thiamine-responsive megaloblastic anemia, Triose phosphate isomerase deficiency, Unstable haemoglobin, Wolfram Syndrome, and X-linked sideroblastic anemia), Gaucher's disease, hemolysis, neutropenia, thrombocytopenia, granulocytopenia, hemophilia, Hodgkin's lymphoma, Non-Hodgkin's lymphoma, B cell chronic lymphoma, Burkitt's lymphoma, Follicular-like lymphoma, diffused large B-cell lymphoma, multiple myeloma, acute myeloid leukemia, pre-B acute lymphocytic leukemia, pre-T acute lymphocytic leukemia, acute promyelocytic leukemia, refractory leukemia, or combinations thereof.

In some embodiments, the disorder characterized by a deficiency of red blood cells results from (partially or fully) one or more of chemotherapy, chemical exposure, radiation therapy, and/or radiation exposure. In some embodiments, a population of red blood cells produced by any method of the present disclosure is co-administered with chemotherapy and/or radiation therapy or one or more protein of interest.

In some embodiments, a population of red blood cells of the present disclosure, and/or produced according to any method of the present disclosure is administered to or transfused into a subject in need thereof, e.g., suffering from a loss of blood. A loss of blood may be the result of for example, internal or external bleeding, hemorrhage, accident, trauma, or surgery, among others.

Treatment with one or more of the population of red blood cells of the present disclosure may also be useful for some infectious diseases associated with hemorrhage, such as but not limited to, families of RNA viruses (Arenaviridae, Bunyaviridae, Filoviridae, and Flaviviridae) that are linked to viral hemorrhagic fever. Examples of viral hemorrhagic fevers including but are not limited to, Lassa fever, Ebola, Marburg, Rift Valley fever, dengue, and yellow fever.

In embodiments where an immediate transfusion is needed, large quantities of red blood cells described herein and/or prepared according to any method of the present disclosure are administered to an individual. In other embodiments, sustained transfusion of the produced population of red blood cells is administered to the individual.

For treatment of some diseases, disorders, and/or conditions it is useful to administer red blood cells adapted to be a delivery system for one or more proteins of interest of the present disclosure. Any methods of adapting red blood cells to be a delivery system for proteins known in the art and disclosed herein may be used. Any disease disorder and/or condition known in the art and disclosed herein that would benefit from treatment with a disclosed protein of interest may be treated with the methods of the present disclosure, including, without limitation, subjects in need of hematopoietic growth factors, acute inflammatory conditions, cytokine storm conditions, clinical signs associate with cytokine storms, cancer, vascular dysregulation (e.g., frost bite, cancer-related vasoconstriction, or rheumatic joints, etc), acute cardiac infarctions, obstetrical uses during child delivery, acute and/or persistent migraine headaches, subjects in need of an immunity booster, subjects at high risk of having clots, subjects at elevated risk for pulmonary embolisms, cardiovascular diseases, immune diseases and/or disorder, and autoimmune diseases and/or disorders.

Exemplary modes of administration of RBCs to a subject for use in the methods described herein include, but are not limited to, injection. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “parenteral administration” and “administered parenterally” as used herein, refer to modes of administration other than enteral and topical administration, usually by injection, and includes, without limitation, intravenous, intraperitoneal, intramuscular, intraarterial, intrathecal, intraventricular, intracapsular, intraorbital, intracardiac, intradermal, transtracheal, subcutaneous, subcuticular, intraarticular, sub capsular, subarachnoid, intraspinal, intracerebro spinal, and intrasternal injection and infusion.

### Kits

Kits for generating a population of RBC from stem cells and/or progenitor cells using inhibitors of SH2B3 are also provided herein. In some embodiments, the kit comprises: (a) a first container comprising a SH2B3 antagonist; (b) a second container comprising necessary growth factors for phase 1 (day 1-day 7) maturation of RBC from stem cells or progenitor cells, comprising: heparin, holo-transferrin, erythropoietin (Epo), stem cell factor (SCF) and IL-3; (c) a third second container comprising necessary growth factors for phase 2 (day 7-day 12) maturation of RBC from stem cells, comprising: heparin, holo-transferrin, erythropoietin (Epo), and stem cell factor (SCF); (d) a fourth container comprising necessary growth factors for phase 3 (day 12-day 18) maturation of RBC from stem cells or progenitor cells comprising; heparin, holo-transferrin and erythropoietin (Epo).

In some embodiments, individual components in the first, second or third or fourth container can be in a form of powder, e.g., lyophilized powder. The powder can be reconstituted upon use. In some embodiments, individual components in the first, second or third container can be in a form of liquid.

In some embodiments, the kit can further comprise one or more containers of basal cell culture medium (e.g., in a form of powder or in liquid). The powder can be reconstituted in an aqueous solution (e.g., water) upon use. Examples of cell culture basal media include, but are not limited to, IMDM, Minimum Essential Medium (MEM), Eagle's Medium, Dulbecco's Modified Eagle Medium (DMEM), Dulbecco's Modified Eagle Medium: Nutrient Mixture F-12 (DMEM F12), F-10 Nutrient Mixture, Ham's F-10 Nutrient Mix, Ham's F12 Nutrient Mixture, Medium 199, RPMI, RPMI 1640, reduced serum medium, basal medium (BME), DMEM/F12 (1:1), and the like, and combinations thereof. In some embodiments, the kit further comprises serum, e.g., human AB plasma, human AB serum. In some embodiments, the kit comprises antiobiotics, e.g., penicillin and/or streptomycin.

In some embodiments, the kit can further comprise one or more vials of pluripotent stem cells or other stem cells, e.g., hematopoietic cells including CD34+ cells.

In some embodiments, the kit can further comprise a cell culture device. Examples of a cell culture device include, but are not limited to, a transwell, a microwell, a microfluidic device, a bioreactor, a culture plate, or any combinations thereof. In some embodiments, the kit can further comprise a microfluidic device. In some embodiments, the microfluidic device can be an organ-on-a-chip device. For example, in one embodiment, the organ-on-a-chip device can be a device as described in the International Pat. App. No. WO 2015/138034, and WO2015/138032 and/or in U.S. Pat. No. US 8,647,861, the contents of each of which are incorporated herein by reference in their entirety. The first channel and the second channel can be of substantially equal (e.g., within 10% or within 5% or less) heights or of different heights. In some embodiments, the height ratio of the first channel to the second channel can range from about 2:1 to about 10:1. In some embodiments, the height ratio of the first channel to the second channel can be about 5:1.

In some embodiments, the kit can further comprise one or more containers each containing a detectable label that specifically binds to a pluripotency marker, or a rbc marker.

In some embodiments, the kit can further comprise instructions for using the kit to perform generation of rbc from pluripotent stem cells or progenitor cells according to the methods as disclosed herein.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

In some embodiments of the present invention may be defined in any of the following numbered paragraphs:
1. A method of producing red blood cells (RBCs) ex vivo from a population of stem cells and/or progenitor cells, the method comprising:
   (i) inhibiting SH2B3 in the population of stem cells and/or progenitor cells;
   (ii) culturing the population of stem cells and/or progenitor cells for a time sufficient to induce differentiation of at least one stem cell or progenitor cell to a RBC; and
   (iii) collecting a population of RBCs.
2. The method of paragraph 1, wherein the inhibiting decreases SH2B3 protein level, SH2B3 mRNA level, SH2B3 protein activity, or combinations thereof.
3. The method of paragraph 1 or 2, wherein the inhibiting comprises contacting the population of stem cells and/or progenitor cells with a genome-editing agent for targeted excision of the SH2B3 gene from at least one stem cell or progenitor cell.
4. The method of paragraph 3, wherein the genome-editing agent is selected from the group consisting of a Zinc-Finger Nuclease (ZFN), a Clustered Regularly Interspaced Short Palindromic Repeats (CRISPR)/CRISPR associated (Cas) system, and a Transcription Activator-Like Effector Nuclease (TALEN).
5. The method of paragraph 3 or 4, wherein the genome-editing agent is present in a vector.
6. The method of paragraph 1 or 2, wherein the inhibiting comprises contacting the population of stem cells and/or progenitor cells with an antagonist of SH2B3.
7. The method of paragraph 6, wherein the antagonist of SH2B3 is selected from the group consisting of an inorganic molecule, an organic molecule, a nucleic acid, a nucleic acid analog or derivative, a peptide, a peptidomimetic, a protein, an antibody or an antigen-binding fragment thereof, and combinations thereof.
8. The method of paragraph 6 or 7, wherein the antagonist of SH2B3 specifically binds to SH2 domain, PH domain, or both the SH2 and PH domains of the SH2B3 protein.
9. The method of any of paragraphs 6-8, wherein the antagonist of SH2B3 is a RNAi agent that inhibits the expression of SH2B3.
10. The method of paragraph 9, wherein the RNAi agent is a siRNA, shRNA, dsRNA that hybridizes to SH2B3 mRNA.
11. The method of any of paragraphs 1-10, wherein the population of stem cells and/or progenitor cells is selected from the group consisting of hematopoietic stem cells, hematopoietic progenitor cells, pluripotent stem cells, induced pluripotent stem cells (iPSCs), embryonic stem cells, and combinations thereof.
12. The method of any of paragraphs 1-11, wherein the method increases the expansion of RBCs from the population of stem cells and/or progenitor cells.
13. The method of any of paragraphs 1-12, wherein the method increases the quality of RBCs.
14. The method of any of paragraphs 1-13, wherein the population of stem cells and/or progenitor cells is of mammalian origin.
15. The method of paragraph 14, wherein the population of stem cells and/or progenitor cells is of human origin.
16. The method of any of paragraph 1-15, wherein the RBCs are isolated by leukocyte filtration or flow cytometric sorting.
17. The method of any of paragraphs 1-16, wherein the population of stem cells and/or progenitor cells is obtained from a donor subject.
18. The method of paragraph 17, wherein the population of stem cells and/or progenitor cells is derived from peripheral blood mononuclear cells, cord blood, bone marrow, cord tissue, or G-CSF mobilize peripheral blood of the donor subject.
19. The method of paragraph 17 or 18, further comprising administering a population of RBCs to a subject in need thereof, wherein the RBCs are produced from the population of stem cells and/or progenitor cells obtained from the donor subject.
20. The method of paragraph 19, wherein the population of stem cells is iPSCs.
21. A population of RBCs produced according to the method of any of paragraphs 1-19.
22. An admixture comprising a population of RBCs and a population of stem cells and/or progenitor cells, wherein the RBCs are produced according to the method of any of paragraphs 1-19.
23. A blood bank comprising a population of RBCs according to paragraph 21.
24. The blood bank of paragraph 23, wherein a plurality of populations of RBCs are prepared for cryogenic storage.
25. A cell culture media comprising a population of stem cells and/or progenitor cells, at least one RBC differentiated from at least one stem cell or progenitor cell, and an antagonist of SH2B3.
26. A method of inducing a population of stem cells and/or progenitor cells to differentiate into red blood cells (RBCs), the method comprising:
   (i) contacting the population of stem cells and/or progenitor cells with an antagonist of SH2B3 and culturing the population of stem cells and/or progenitor cells for a time sufficient to induce the differentiation of at least one stem cell or progenitor cell into a RBC, wherein the antagonist of SH2B3 decreases the activity of the SH2B3 protein or decreases SH2B3 mRNA or protein levels; and
   (ii) collecting a population of RBCs.
27. A method of inducing a population of stem cells and/or progenitor cells to differentiate into red blood cells (RBCs), the method comprising:
   (i) contacting the population of stem cells and/or progenitor cells with a genome-editing agent and culturing the population of stem cells and/or progenitor cells for a time sufficient to induce the differentiation of at least one stem cell or progenitor cell into a RBC, wherein the genome-editing agent excises the SH2B3 gene from at least one stem cell or progenitor cell; and
   (ii) collecting a population of RBCs.
28. A method of administering a population of RBCs to a subject, comprising administering an effective amount of RBCs to the subject, wherein the RBCs have been contacted ex vivo or in vitro with an effective amount of an antagonist of SH2B3, wherein the antagonist of SH2B3 decreases the activity of the SH2B3 protein or decreases SH2B3 mRNA or protein levels.
29. A method of administering a population of RBCs to a subject, comprising administering an effective amount of RBCs to the subject, wherein the RBCs are produced from a population of stem cells and/or progenitor cells having been contacted ex vivo or in vitro with an effective amount of a genome-editing agent, wherein the genome-editing agent excises the SH2B3 gene from at least one stem cell or progenitor cell.
30. A cell culture media comprising: a population of stem cells and/or progenitor cells, and at least one antagonist of SH2B3, wherein the cell culture media is selected from;
   a. phase 1 (day 1-7) differentation media comprising heparin, holo-transferrin, erythropoietin (Epo), stem cell factor (SCF) and IL-3); or
   b. phase 2 (day 7-day 12) differentiation media comprising comprising heparin, holo-transferrin, erythropoietin (Epo), and stem cell factor (SCF); or
   c. phase 3 (day 12-day 18) differentiation media comprising heparin, holo-transferrin and erythropoietin (Epo).
31. The culture media of paragraph 30, further comprising at least one RBC differentiated from a stem cell and/or progenitor cell.
32. A cell culture media comprising: at least one RBC and at least one antagonist of SH2B3, wherein the cell culture media is selected from;
   a. phase 1 (day 1-7) differentation media comprising heparin, holo-transferrin, erythropoietin (Epo), stem cell factor (SCF) and IL-3); or
   b. phase 2 (day 7-day 12) differentiation media comprising comprising heparin, holo-transferrin, erythropoietin (Epo), and stem cell factor (SCF); or
   c. phase 3 (day 12-day 18) differentiation media comprising heparin, holo-transferrin and erythropoietin (Epo).
33. The method of any of paragraphs 1-19, wherein the population of stem cells and/or progenitor cells is obtained from a human subject who is a Type O blood type.
34. The method of paragraph 33, wherein the human subject has a Type O− (Type O, Rh−) blood type.

Although any known methods, devices, and materials may be used in the practice or testing of the invention, the methods, devices, and materials in this regard are described herein.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### EXAMPLES

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The technology described herein is further illustrated by the following examples which in no way should be construed as being further limiting.

### Materials and Methods

293T Cell Culture and Lentiviral Production

293T cells were kept in DMEM supplemented with 10% FBS and 1% penicillin/streptomycin at 30%-90% confluency. For lentiviral production, 293T cells were transfected with the constructs described below along with the VSV-G envelope and pdA8.9 packaging vector using the Fugene 6 reagent (Roche) according to the manufacturer's protocol. Medium was changed to phase 1 primary cell culture medium (without EPO, IL-3 and SCF) the following day and viral supernatant collected and filtered at 45 microns at 48 h post transfection. TF-1 human erythroid cells were cultured in RPMI media supplemented with 10% FBS and 2 ng/ml GM-CSF for maintenance and 1% penicillin/streptomycin. For cytokine starvation, all cytokines were removed and cells were maintained in FBS only overnight. EPO and SCF were added at time 0 and then subsequent time points were collected for analysis.

Isolation and Source of Primary CD34+ Cells.

CD34+ cells from G-CSF mobilized peripheral blood, bone marrow, or cord blood was purified by positive magnetic selection using an Ultrapure Microbead Kit (Miltenyi Biotech) according to the manufacturer's protocol following mononuclear cell purification on a Ficoll Density Gradient. At least 95% purity was reached as assessed by post-purification flow cytometry with a PE conjugated anti-human CD34 antibody (8012-0349, eBioscience) as described below.

Primary Cell Culture and Lentiviral Infection

Cells were differentiated into mature RBCs utilizing a three phase culture protocol. In phase 1 (day 0-7) cells were cultured at a density of 10⁵-10⁶ cells per milliliter (mL) in IMDM supplemented with 2% human AB plasma, 3% human AB serum, 1% penicillin/streptomycin, 3 IU/mL heparin, 10 ug/mL insulin, 200 ug/mL holo-transferrin, 1 IU erythropoietin (Epo), 10 ng/mL stem cell factor (SCF) and 1 ng/mL IL-3. In phase 2 (day 7-12), IL-3 was omitted from the medium. In phase 3 (day 12-18), cells were cultured at a density of 10⁶ cells per milliliter, with both IL-3 and SCF omitted from the medium and the holo-transferrin concentration was increased to 1 mg/mL. Cells were cultured at 37° C. and 5% CO₂. For lentiviral infection, medium was changed on day 1 to viral supernatant along with 8 ug/mL polybrene and EPO, IL-3, and SCF and spun at 2000 rpm for 90 min at room temperature. After 12 hours of infection, cells were placed in 1 ug/mL puromycin for 36-48 hours.

As an alternative approach, the inventors also developed an adaptation of the erythroid culture method described above by adding an expansion phase lasting a total of 5 days prior to initiation of erythroid differentiation and starting phase 1 of the culture. Recent work has shown that such an approach can result in significant expansion of erythroid cells and can improve mature RBC production (Lee et al., 2015). Similar to those reported findings, the inventors discovered that this allows for increased expansion and comparable differentiation as our standard 3-phase culture method. During the expansion phase >85% of cells remain CD34+. The expansion medium was composed of StemSpan II serum free expansion medium (Stem Cell Technologies) and 1× CC100 cytokine mix composed of FLT3L, IL-3, IL-6, and SCF (Stem Cell Technologies), as we have described previously (Sankaran et al., 2011; Sankaran et al., 2008). Media was changed every 2-3 days and cells were maintained at a density between 105-106 cells per milliliter (mL). Lentiviral infections were carried out on day 2 of this expansion phase and selection occurred, as described above.

Western Blotting

1-2×10⁶ cells were harvested on day 9 of culture, washed twice in PBS, and resuspended in RIPA buffer (50 mM Tris-HCl, 150 mM NaCl, 0.1% SDS, 1% NP-40, 0.25% sodium deoxycholate) and lysed in the presence of sodium orthovanadate, protease inhibitor cocktail, and PMSF for 30 min on ice with intermittent mixing. After removal of cell debris by centrifugation, the supernatant was transferred to a new tube and the protein concentration was determined mixed with Loading Dye and incubated at 95° C. for 5 min. SDS-PAGE analysis was performed using precast gels (BioRad) and 1% Tris-Glycine-SDS running buffer. Protein was transferred on to nitrocellulose membranes in 10% methanol in tris/glycine. Membranes were blocked with TBS-T supplemented with 3% BSA for 1 h and incubated with the primary antibody for 1 h at room temperature or overnight at 4° C. Subsequent to washing four times in TBS-T for 10 min the membrane was incubated with the secondary antibody for 1 h at room temperature. After another 40 minutes of washing in TBS-T and 5 min incubation in ECL-substrate (BioRad), proteins were visualized by scientific imaging film exposed to the treated membrane. LNK/SH2B3 sheep polyclonal antibody (AF5888, R&D Systems) and GAPDH mouse monoclonal antibody (6C5, sc-32233, Santa Cruz) served as primary, goat anti-mouse (170-5047, BIORAD) and donkey anti-sheep (713-035-147, Jackson Immuno Research) HRP-coupled antibodies served as secondary reagents. For the Western blots shown, cells were typically harvested at day 9 of culture.

Flow Cytometry

Cells were harvested and washed twice in FACS Buffer (PBS+3% BSA) for 5 min at 300×g. For differentiation analysis cells were subsequently stained for 10-15 min on ice with the following antibodies at a 1:20 dilution in FACS Buffer: APC conjugated Anti-Human CD235a (17-9987, eBioscience), FITC conjugated Anti-Human CD71 (11-0719, eBioscience), PE conjugated Anti-Human CD41a (12-0419, eBioscience) and PE conjugated Anti-Human CD11b (12-0118, eBioscience). Where indicated cells were additionally stained with 1 ug/mL Hoechst 33342 (Life Technologies) and incubation was performed for 20 min at room temperature. Unbound antibodies were removed by washing twice with FACS Buffer, afterwards the pellet was resuspended in FACS Buffer supplemented with propidium iodide (PI) stain (00-6990, eBioscience) at a 1:20 dilution and transferred into FACS tubes for subsequent analysis on a FACS Canto II or LSR II (BD Bioscience) Flow Cytometer. Cells were analysed with Flow Joe (Tristar) and FACS Diva (BD Bioscience) software package, PI positive cells, cell debris, and aggregates were gated out of the analysis.

May-Grünwald-Giemsa Staining

Approximately 50,000-200,000 cells were harvested, washed once at 300×g for 5 min, resuspended in 200 μL of FACS Buffer, and spun onto poly-L-lysine coated microscope slides with a Shandon 4 (Thermo Scientific) cytocentrifuge at 300 rpm for 4 min. When visibly dry slides were transferred into May-Grünwald solution (Sigma-Aldrich) for 5 min, rinsed 4 times for each 30 s in H₂O, and transferred to Giemsa solution (Sigma-Aldrich) for 15 min. Slides were washed as described above and after drying mounted with coverslips and examined the following day. All images shown were taken with AxioVision software (Zeiss) at 100× magnification.

Constructs

shRNA constructs targeting SH2B3 were used with the following sequences: sh83 (CCGGCCTGACAACCTTTACACCTTTCTCGAGAAAGGTGTAAAGGTTGTCAGGTTTTTG) (SEQ ID NO: 1) and sh84 (CCGGGCCTGACAACCTTTACACCTTCTCGAGAAGGTGTAAAGGTTGTCAGGCTTTTTG) (SEQ ID NO: 2). Both constructs were in the pLKO.1-puro lentiviral vector. The lentiviral vectors pLKO-GFP and pLKO.1-puro Luciferase served as controls (the RNAi consortium of the Broad Institute of MIT and Harvard).

Cell Division Analysis

On day 5 of differentiation an equal number of SH2B3-KD and control cells were labeled with PKH26 (PKH26GL, Sigma Aldrich) according to the manufacturer's protocol. In brief, cells were washed in IMDM (no addition of serum) and resuspended in diluent C Immediately prior to staining, a 4×10⁻⁶ M PKH dye solution was prepared and rapidly added to the cell suspension at equal volumes. After 5 minutes of incubation the staining reaction was stopped by addition of human AB serum and unbound dye was removed by washing 3 times in phase 1 medium without EPO, SCF, or IL-3. Mean fluorescent intensities (MFI) of PKH26 were obtained immediately after labeling as well as on day 7 and 9 of differentiation on a Canto 2 (BD Bioscience) flow cytometer. The number of cell divisions, x, was approximated as described earlier (Sankaran et al. 2012): x=(log((MFI 0h)/(MFI yh)))/(log 2)).

Eosin-5-Maleimide (EMA)—Binding Test

2-4×10⁶ cells were harvested washed with PBS and subsequently labeled with eosin-5-maleimide (Life Technologies) as recently described by King et al. (2000). In brief, pelleted mature RBCs were resuspended in 25 uL of 0.5 mg/mL EMA and incubated for 1 hour in the dark with careful intermittent mixing. Unbound dye was removed by washing 3 times in PBS+3% BSA, afterwards cells were incubated for another 10 min with APC-conjugated anti-human CD235a (eBioscience) at a 1:20 dilution at room temperature. After removal of unbound antibody as described above, pellets were resuspended in FACS-Buffer and samples were analysed on a Canto 2 (BD Bioscience) flow cytometer, the emission for EMA was detected in the PE channel at 564-606 nm.

Advia

Approximately 5×10⁷ cells were harvested on day 17 of differentiation, spun, and resuspended in 300 uL PBS and analysed on an Advia 2120i (Siemens Healthcare).

Gene Expression Analysis

Microarrays (GeneChip Human Gene 2.0 ST Arrays, Affymetrix) were performed on erythroblasts (day 7 of differentiation) for SH2B3 KD (sh83 and sh84) and control samples (shLuc). Raw files were processed and normalized using the RMA algorithm from the oligo package in R 3.2 (Carvalho and Irizarry, 2010). Differential expression analyses were conducted using limma (Ritchie et al., 2015). A heatmap is displayed for normalized expression values (Z-score) of genes that were significantly up-regulated in SH2B3 KD at a log 2 fold change greater than 0.5 and a p-value threshold of 0.001. Gene set enrichment analysis (GSEA) was performed by comparing combined SH2B3 KD to control samples with standard options, except geneset permutation was used instead of sample permutation because there were n=3 samples for each group(Subramanian et al., 2005). We derived the erythroid differentiation signature gene set by identifying the top 200 genes that were expressed significantly higher in intermediate erythroblasts (CD71+/CD235a+) compared to colony forming unit erythroid cells (CD71+/CD235a−) (Merryweather-Clarke et al., 2011). All data has been deposited in the Gene Expression Omnibus (GEO, http://www.ncbi.nlm.nih.gov/geo/) under accession GSE67219.

CRISPR/Cas9 Genome Editing in human ESCs

For SH2B3 knockout in human embryonic stem cell line (hESCs; HUES9), a plasmid co-expressing a codon-optimized Streptococcus pyogenes Cas9 and EGFP with the CAG promoter and a plasmid expressing the guide RNA (gRNA) with the human U6 promoter were co-transfected. To target SH2B3 the following protospacer and protospacer-adjacent motif (PAM) were used: 5′-GCTCCAGCATCCAGGAGGTC-CGG-3′ (SEQ ID NO: 4)

Alternatively, gRNA was used: 5′-GTGTGCACCACCGGACCTCCTGG-3′ (SEQ ID NO: 3)

hESCs were initially cultured in mTeSR1 supplemented with penicillin/streptomycin on Geltrex matrix coated tissue culture plates. Cells were dissociated using Accutase at the presence of ROCKi and subsequently 10 million single cells were electroporated with 25 ug of each plasmid in a single cuvette. Cells were replated and after 48-72 hours treated with accutase, collected, and resuspended in PBS. EGFP expressing cells were sorted by FACS (FACSAria II, BD Bioscience) and plated at 15,000 cells/plate in growth medium and allowed to recover for 7-10 days. Afterwards single colonies were manually picked and individually replated in wells of 96-well plates, grown near confluence for another 7 days, and accutase treated to create a frozen stock. PCR screening was employed to identify colonies with compound heterozygous or homozygous frameshift deletions in SH2B3. The lines BB5, DB3, and BF4 had homozygous or compound heterozygous frameshift mutations creating knockout of SH2B3 and were used for the differentiation analyses. The inventors used isogenic lines BA3, BA6, and BB1 with intact wild-type SH2B3 alleles as controls for the differentiation analyses.

Maintenance and Erythroid Differentiation of hES Cells

hESCs were maintained in six-well tissue culture plates containing 0.75-1.0×10⁶ mouse embryonic fibroblasts (MEFs) in daily exchanged HES medium containing DMEM supplemented with 20% KSR, 100 uL nonessential amino acids solution, 50 U/mL penicillin, 50 g/mL streptomycin, 2 mM glutamine, 1 mM sodium pyruvate, 0.075 sodium bicarbonate, and 0.1 mM beta-mercaptophenol at a maximum confluency of 90% at 37° C. and 5% CO₂. For passaging, cells were treated with TrypLE, rinsed with medium and after addition of 10 uM ROCKi to prevent apoptosis colonies were scraped into small clumps, aspirated and subsequent to mixing replated on MEF containing plates at an overall dilution of 1/7 in HES medium. For MEF depletion, hESCs were harvested as described above and replated onto Matrigel coated dishes at a density of 1-1.5×10⁵ per milliliter in HES medium containing 5 ng/mL bFGF and 10 uM ROCKi at 37° C., 5% CO₂, 5% O₂ and 90% N₂. Every 24 h cells were fed by replacing with fresh medium excluding ROCKi. Subsequent to reaching approximately 70% confluence 2-3 daysfollowing MEF depletion, differentiation to hematopoietic progenitor cells (HPCs) was initiated by culturing cells on a daily feeding regime with sequential medium and cytokine changes depicted in the Table 3. Cells were kept in the Matrigel coated dishes at 37° C., 5% CO₂, 5% O₂ and 90% N₂ for 8-9 days until appearing bright round loosely adherent HPCs were collected by carefully removing the supernatant. Subsequently an aliquot of cells was taken and flow cytometry was performed as described below to assess for the presence CD41a and CD235a double positive multipotent HPCs. For further differentiation into primitive erythoblasts HPCs were spun at 335×g, resuspended in erythroid expansion medium (EEM) containing SFD supplemented with 50 ug/ml Ascorbic Acid, 0.45 mM MTG, 50 ng/ml SCF and 2 units/ml EPO. Media was exchanged every 1-2 days. Cells were carefully counted with a hematocytometer and analyzed by flow cytometry at indicated time points.

TABLE 3
Timetable of Differentiation of PSCs into
HPCs showing culture medium and supplements

| Day | Medium* | BMP4*** | VEGF | Wnt3a | BFGF | SCF | Flt3L | IL-6 |
|---|---|---|---|---|---|---|---|---|
| 0-1 | RPMI | 5 | 50 | 25 | | | | |
| 2 | RPMI | 5 | 50 | | 20 | | | |
| 3 | SP34 | 5 | 50 | | 20 | | | |
| 4-5 | SP34 | | 15 | | 5 | | | |
| 6 | IMDM** | | 50 | | 50 | 50 | 5 | |
| 7-9 | IMDM** | | 50 | | 50 | 50 | 5 | 10 |

*all supplemented with 2 mM Glutamine, 50 ug/mL ascorbic acid and 4 * 10⁻⁴ monothioglycerol
**supplemented with 0.5% N₂, 1% B27 without Vitamin A and 0.05% BSA
***cytokine concentrations in ng/mL

Statistical Analysis

All pairwise comparisons were assessed by an unpaired two-tailed Student's t-test. Results were considered significant if the P value was below 0.05.

### Example 1

Multipotent and pluripotent stem cells are potential sources for cell and tissue replacement therapies. For example, stem cell-derived red blood cells (RBCs) are a potential alternative to donated blood, but yield and quality remain a challenge. Here, the inventors demonstrate that application of insight from human population genetic studies can enhance RBC production from stem cells. The inventors have discovered that the SH2B3 gene encodes a negative regulator of cytokine signaling and naturally occurring loss-of-function variants in this gene increase RBC counts in vivo. Targeted suppression of SH2B3 in primary human hematopoietic stem and progenitor cells enhanced the maturation and overall yield of in-vitro-derived RBCs. Moreover, the inventors demonstrate that inactivation of SH2B3 by CRISPR/Cas9 genome editing in human pluripotent stem cells allowed enhanced erythroid cell expansion with preserved differentiation. Thus, the inventors have demonstrated that genome editing approaches can be used to improve cell and tissue production for regenerative medicine.

There is significant excitement regarding the use of stem cells as a source for cell replacement therapies (Fox et al., 2014). In vitro differentiated red blood cells (RBCs) from hematopoietic stem and progenitor cells (HSPCs) have been successfully transfused into recipients in clinical trials (Giarratana et al., 2011; Migliaccio et al., 2012), and similar approaches using pluripotent stem cells (PSCs), including human embryonic stem cells (hESCs) and induced PSCs (iPSCs), are under investigation (Kobari et al., 2012; Slukvin, 2013; Sturgeon et al., 2013). However, utilizing these stem cell sources for RBC production is limited by low yields of fully mature cells, making the process costly and inefficient (Migliaccio et al., 2012; Rousseau et al., 2014). Overcoming these major limitations could enhance the ability to manage the blood supply (Williamson and Devine, 2013).

Numerous ongoing efforts focus on optimizing culture methods to improve RBC production from various stem cell sources (Huang et al., 2015; Rousseau et al., 2014). A complementary approach is to investigate whether human genes that regulate RBC production in vivo can be manipulated to enhance this process in vitro. Recent genome-wide association studies (GWASs) of RBC traits and subsequent fine-mapping identified a common coding single nucleotide polymorphism (SNP) in the SH2B3 gene (rs3184504), which results in a R262W substitution, that is significantly associated with hemoglobin levels and RBC counts in vivo (van der Harst et al., 2012). SH2B3 is an SH2- and PH-domain-containing protein that negatively regulates hematopoietic cytokine signaling. Mice with null mutations in SH2B3 have normal hemoglobin and RBC counts, suggesting human-specific functions in RBC production (erythropoiesis) (Bersenev et al., 2008; Velazquez et al., 2002). The rs3184504 variant associated with increased RBC counts and hemoglobin levels is thought to be a hypomorphic allele (McMullin et al., 2011; van der Harst et al., 2012). Consistent with this possibility, rare loss-of-function (LoF) SH2B3 alleles are associated with greater elevations in the RBC count and hemoglobin level (erythrocytosis) (Lasho et al., 2010; Spolverini et al., 2013). To extend this observation to a population-based cohort, the inventors examined a group of 4,678 individuals subjected to whole-exome sequencing and observed higher hemoglobin/hematocrit values among individuals with rare putative damaging missense or LoF variants in SH2B3 (Data not shown).

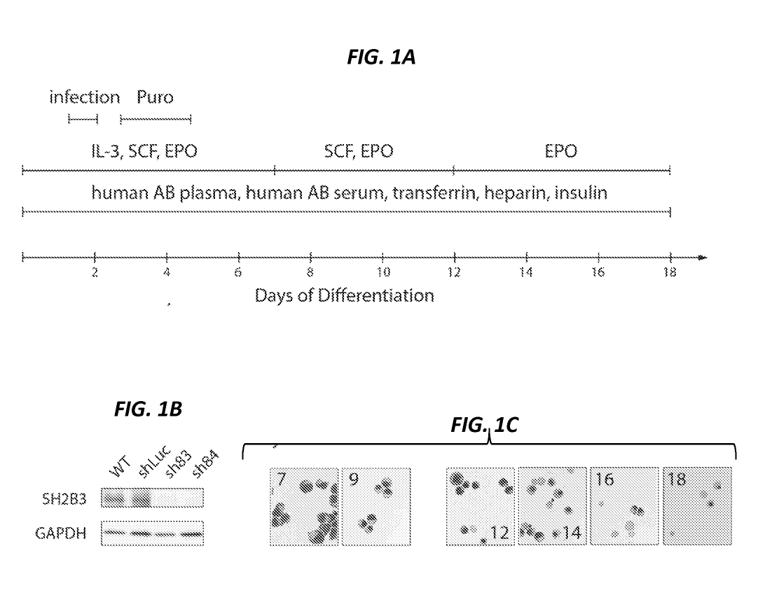

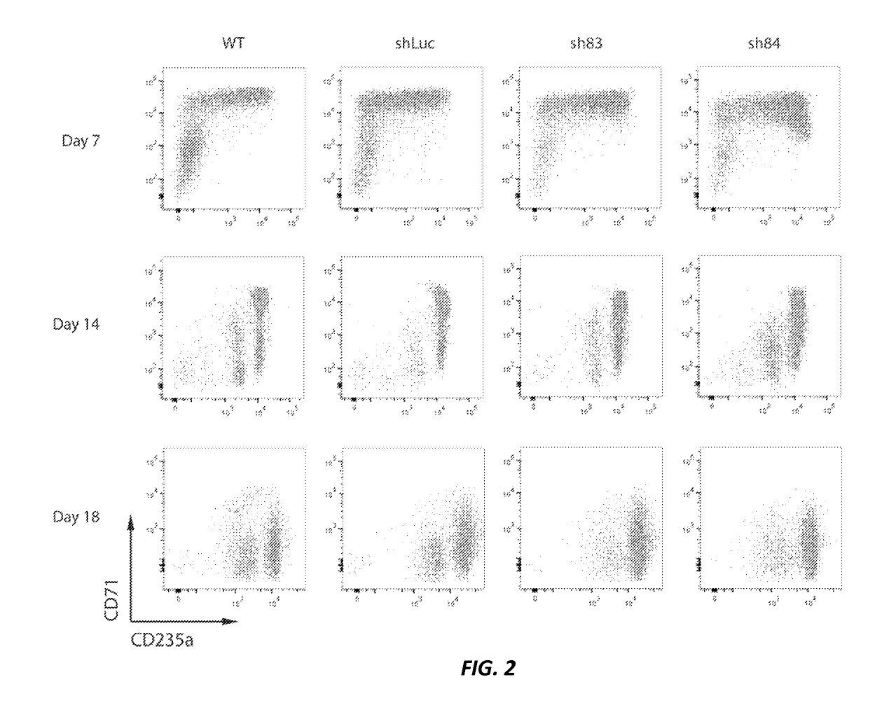

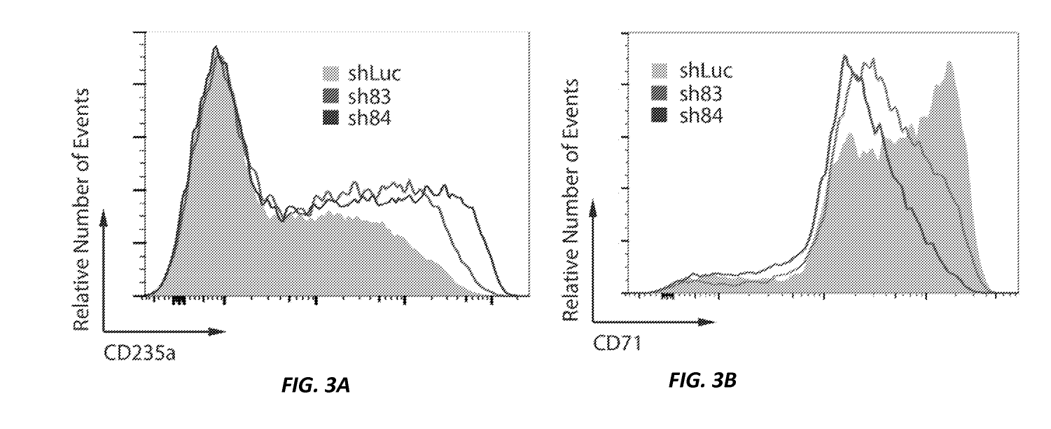

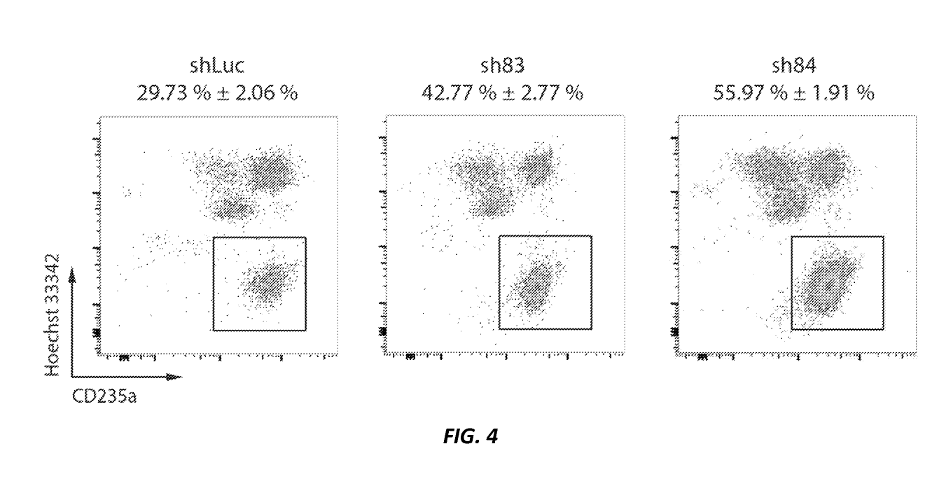

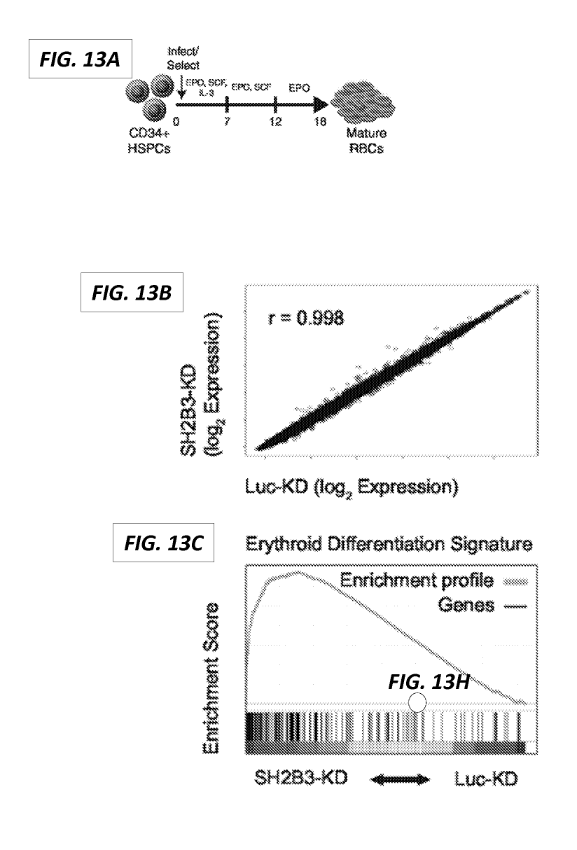

The inventors next tested whether suppression of SH2B3 can enhance erythropoiesis in vitro. The inventors used lentiviral vectors to express short hairpin RNAs (shRNAs) targeting SH2B3 in adult CD34+ HSPCs that were induced to undergo erythroid differentiation (FIG. 13A and FIG. 1B). The SH2B3 knock-out (e.g. Loss of function or LoF) cultures with sh83 (SEQ ID NO: 1) and sh84 (SEQ ID NO: 2) shRNAs differentiated similar to controls, although the erythroid maturation as determined by cell surface phenotyping (loss of CD71, as well as acquisition of CD235a) occurred earlier (FIGS. 3A and 3B). Interestingly, the inventors discovered a 1.6- to 2-fold increase in enucleation in SH2B3 knock-out erythroblasts (FIG. 4). These observations were confirmed by analysis of cell morphology that demonstrated improved maturation, better hemoglobinization, and greater enucleation (FIG. 5). Gene expression analysis of erythroblasts from the SH2B3 knock-out cultures showed a globally similar profile with those of controls (FIG. 13C), with enhanced expression of genes associated with terminal erythroid maturation (FIG. 13D). The inventors maintained the cultures following enucleation for several days and did not observe any improvement in maturation in the controls in comparison with the SH2B3 knock-out cultures.

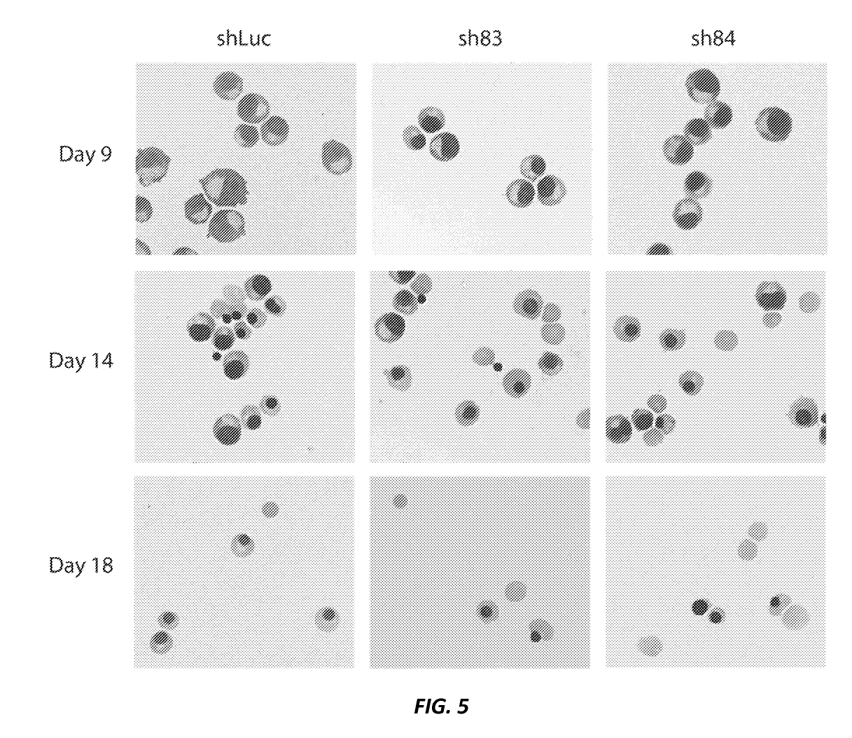

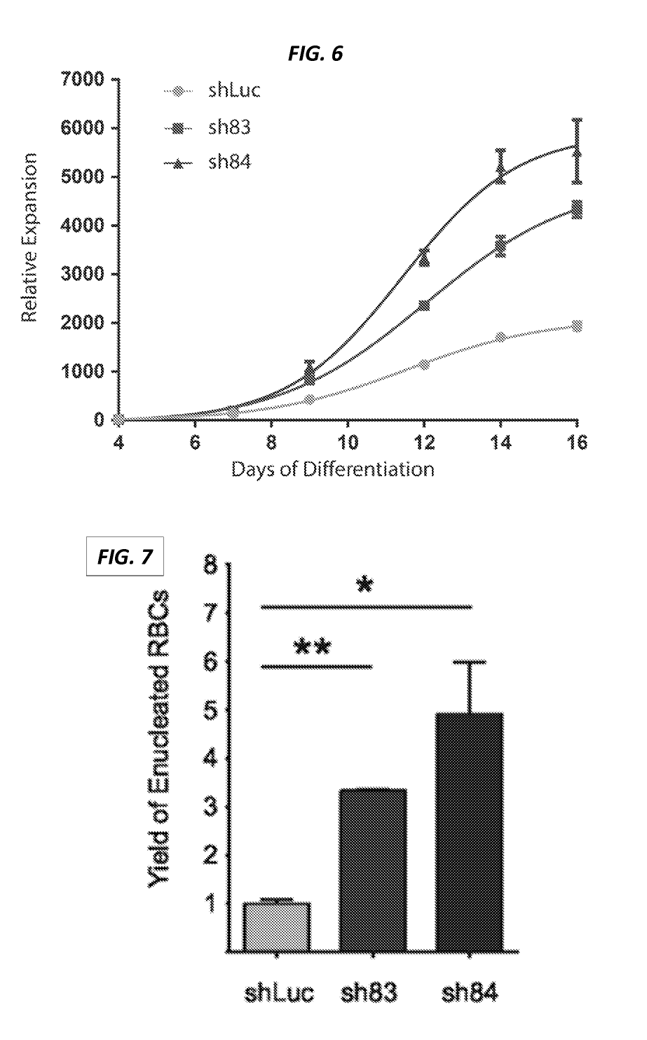

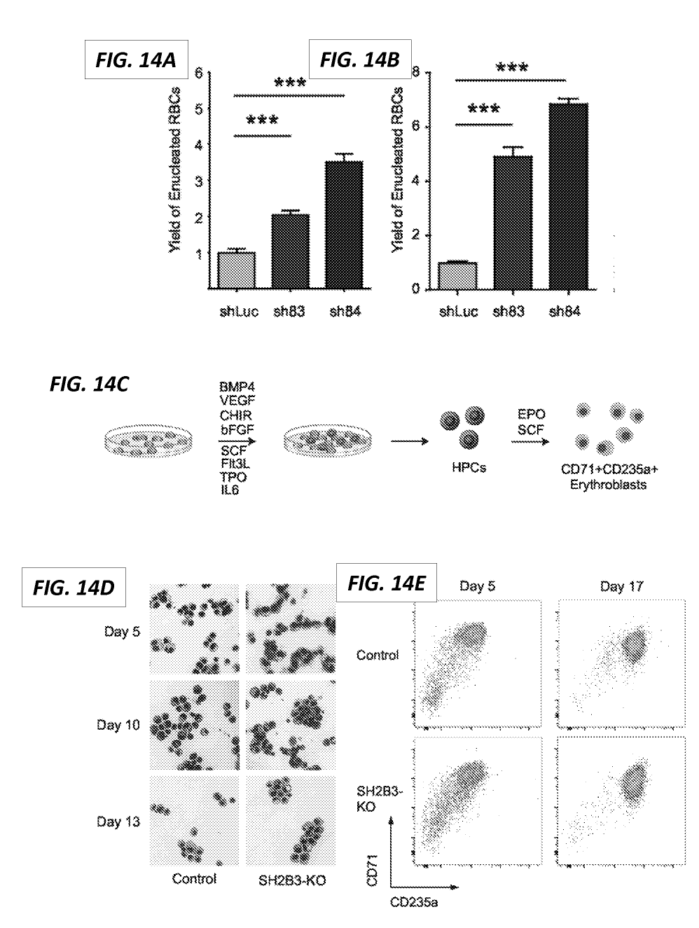

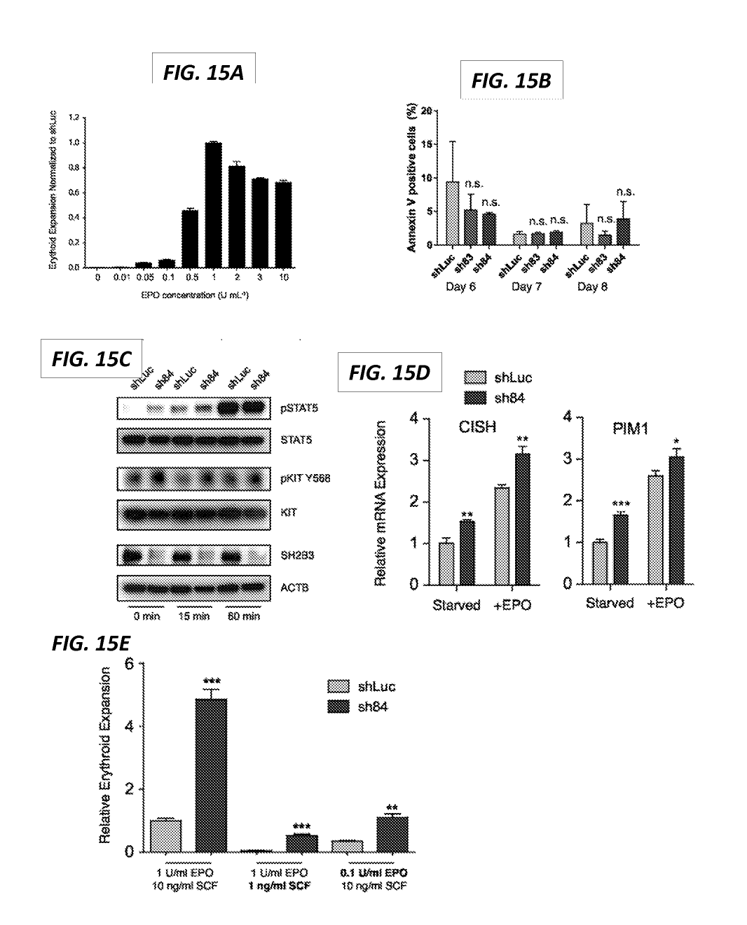

Since cells with SH2B3 knock-out appeared to undergo erythroid differentiation more readily, the inventors assessed if their proliferative potential was restrained, which could compromise RBC yield. Surprisingly, adult HSPCs with SH2B3 knock-out expanded to a significantly greater extent when compared with controls (FIG. 7 and FIG. 6). The overall increase in expansion occurred over multiple stages of the differentiation process, suggesting that SH2B3 suppression augments multiple signaling pathways at different stages of differentiation (FIG. 6). The observed effect was not due to suboptimal cytokine concentrations, as the cells were cultured in erythropoietin (EPO) and other cytokine concentrations where they demonstrated maximal expansion (FIG. 15A) (Abkowitz et al., 1991). Overall, the yield of mature RBCs was expanded 3- to 5-fold by suppression of SH2B3 (FIG. 7). The inventors performed a similar knockdown of SH2B3 in CD34+ cord blood HSPCs, which are known to exhibit over 10-fold greater expansion during erythroid differentiation compared to adult HSPCs (Giarratana et al., 2011; Migliaccio et al., 2012; Rousseau et al., 2014). In these cultures, SH2B3 knock-out further enhanced enucleated RBC yield by 2- to 4-fold (FIG. 14A). In addition, the inventors also utilized an adaptation of a recently described culture approach incorporating a progenitor expansion step with adult CD34+ HSPCs (Lee et al., 2015; Ludwig et al., 2014; Sankaran et al., 2011). In this case, the inventors discovered an even greater increase in expansion and overall yield of RBCs of 5- to 7-fold (FIG. 14B), demonstrating the generalizability of SH2B3 suppression to augment RBC production from HSPCs.

### Example 2

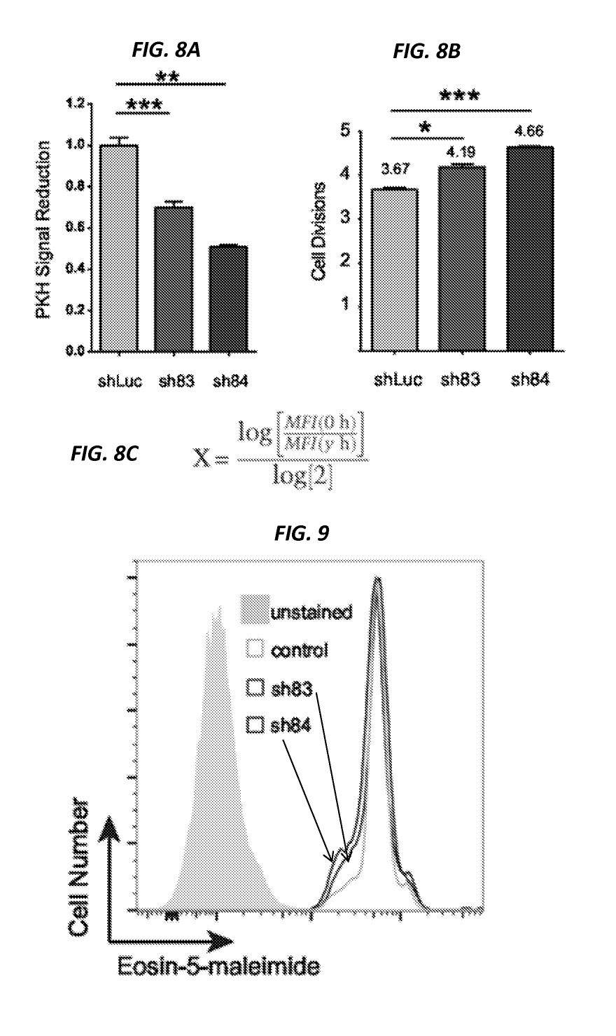

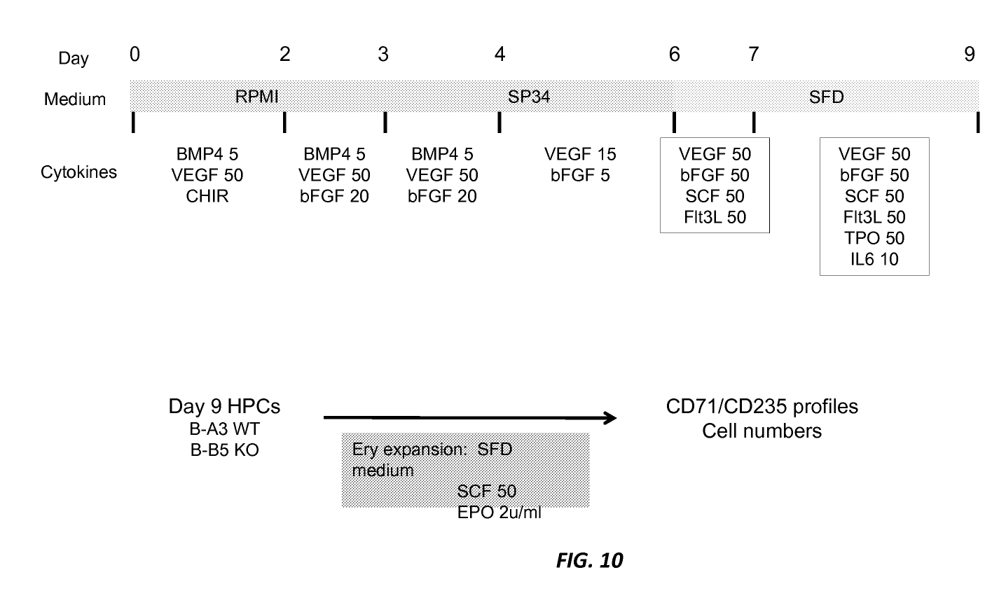

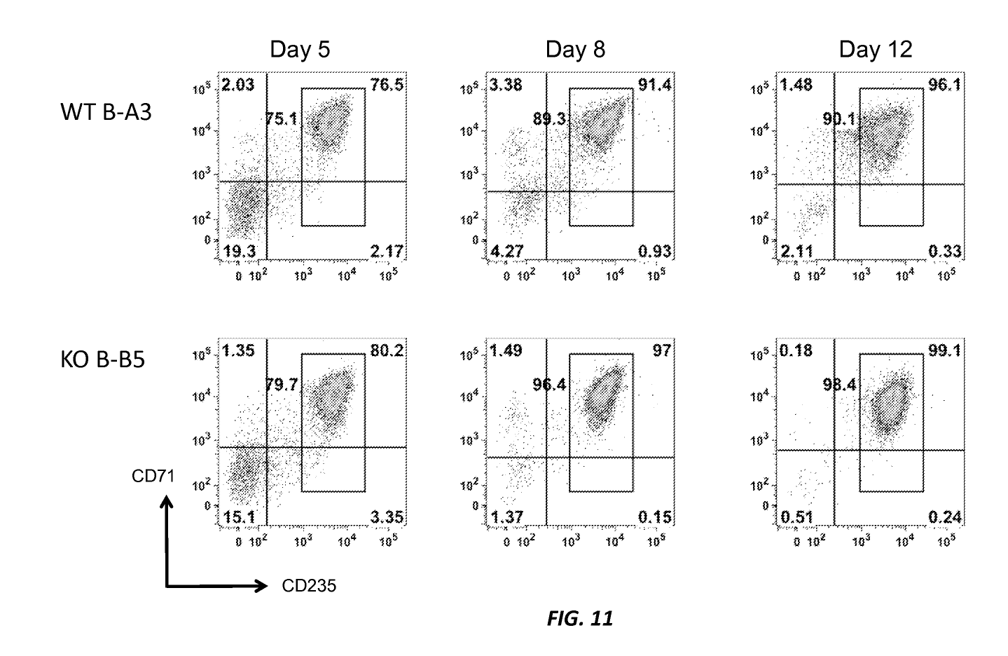

The inventors next assessed the mechanisms through which suppression of SH2B3 is able to augment erythroid differentiation and expansion. The fraction of apoptotic cells, as assessed by annexin V staining, was not altered by SH2B3 suppression in adult HSPCs (FIG. 15B). Cells with SH2B3 knock out underwent 0.5-1 additional cell divisions over the 4 day period of differentiation examined (FIGS. 8A, 8B) (Sankaran et al., 2012). SH2B3 negatively regulates signaling downstream of multiple cell surface signaling receptors implicated in erythro-poiesis, including EPO, KIT, and integrin receptors (Gery and Koeffler, 2013; McMullin et al., 2011). Accordingly, phosphorylation of STAT5 and KIT receptor and the expression of early EPO responsive genes were enhanced by SH2B3 suppression (FIG. 15C and FIG. 15D) (Moraga et al., 2015). The inventors verified the importance of both the EPO and KIT/SCF pathways for the observed augmentation upon SH2B3 suppression by limiting the concentration of both cytokines in the HSPC differentiation cultures (FIG. 15E). SH2B3 suppression allowed erythroid expansion similar to the baseline control, with reduced levels of either EPO or SCF, highlighting the role of SH2B3 in both pathways in primary erythroid cells. Thus, SH2B3 knock-out or inhibition appears to facilitate erythroid expansion and maturation by augmenting both the EPO and KIT signaling pathways.

### Example 3

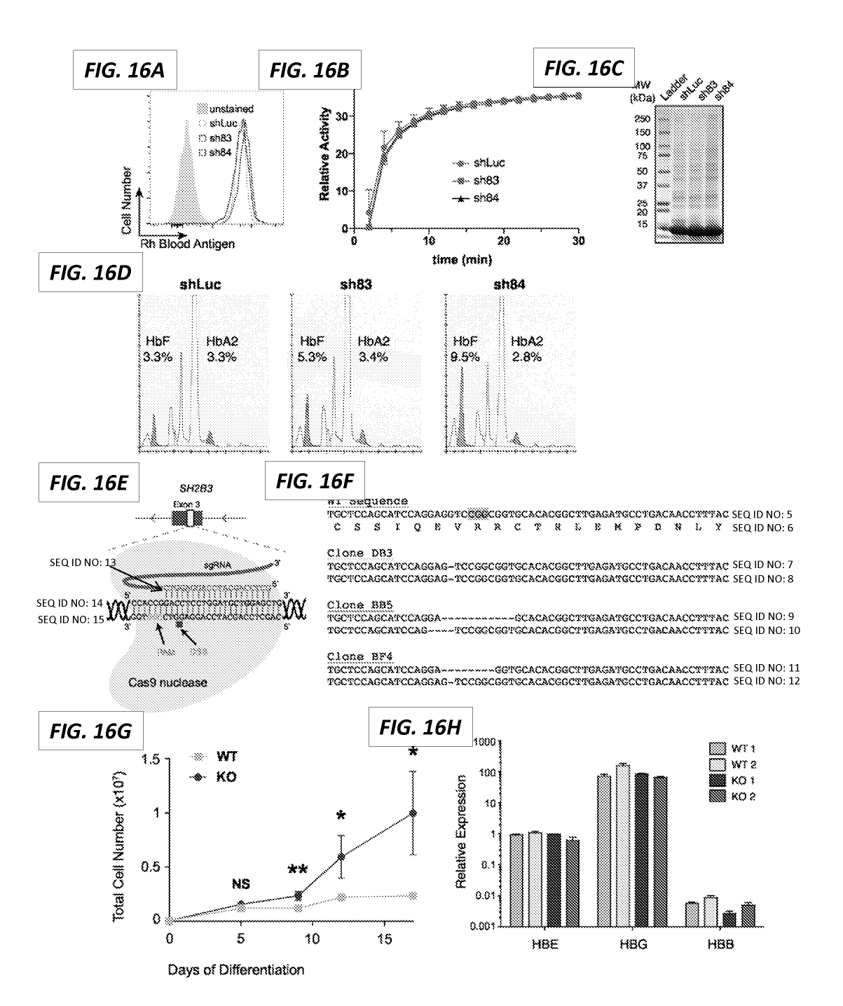

Since the inventors discovered that the production of erythroid cells from human HSPCs appeared to be increased by SH2B3 knockout or inhibition, the inventors examined whether the RBCs produced in vitro were altered from those of controls. The average volume of RBCs was similar between those arising from control and SH2B3 knock-out cultures (average of 108.7 in controls, compared with 110.1 and 104.7 femtoliters in the sh83 (SEQ ID NO: 1) and sh84 (SEQ ID NO: 2) SH2B3 shRNAs). These values were within the normal range of 103-126 femtoliters for reticulocytes (immature RBCs, equivalent to those produced in the culture) found in human adults (d'Onofrio et al., 1995). Moreover, SH2B3 shRNA knock-out improved hemoglobinization with the mean hemoglobin content being 27 and 26.7 picograms (pg) in sh83- and sh84-treated cells, respectively, compared with 25.4 in controls (human adult reticulocytes generally have concentrations of 25.9-30.6 pg; d′Onofrio et al., 1995). Disorders of the RBC membrane, such as hereditary spherocytosis, are clinically diagnosed by variation in binding of the dye eosin-5-maleimide (EMA) (Perrotta et al., 2008). The inventors discovered that EMA staining was comparable between the SH2B3 LoF RBCs and controls, suggesting normal formation of the RBC cytoskeleton (FIG. 9). In addition, surface expression of RBC antigens, including the Rh blood group (FIG. 16A) and CD44, and activity of the cytoplasmic pyruvate kinase enzyme were similar between the SH2B3 LoF RBCs and controls (FIG. 16B). RBC ghosts from the SH2B3 knock-out and control cells showed identical protein patterns, providing an independent metric for normal RBC maturation (FIG. 16C). The inventors also discovered normal hemoglobin subunits present in the RBCs with a small increase in fetal hemoglobin (HbF) production with SH2B3 shRNA knock-out, consistent with the known increase in HbF observed with acceleration at the early stages of erythropoiesis (FIG. 16D) (Sankaran and Orkin, 2013). Collectively, these results demonstrate that RBC physiology was not impaired in cells derived from SH2B3 knock-out HSPCs, despite a marked improvement in differentiation and expansion.

Overall, the inventors have demonstrated reducing SH2B3 expression could improve in vitro production of RBCs for transfusion purposes. While viral-mediated shRNA delivery to primary CD3430 HSPCs may be impractical on a large scale, it should be possible to disrupt the SH2B3 gene in self-renewing cell lines that can be used for RBC production (Rousseau et al., 2014). Despite promising recent advances (Hirose et al., 2013; Huang et al., 2014; Kurita et al., 2013), no immortalized human cell lines allow consistent differentiation into mature RBCs. However, PSCs can be readily differentiated toward the erythroid lineage with well-established protocols (Huang et al., 2015; Kobari et al., 2012; Mills et al., 2014; Slukvin, 2013; Sturgeon et al., 2014) (FIG. 16E).

### Example 4

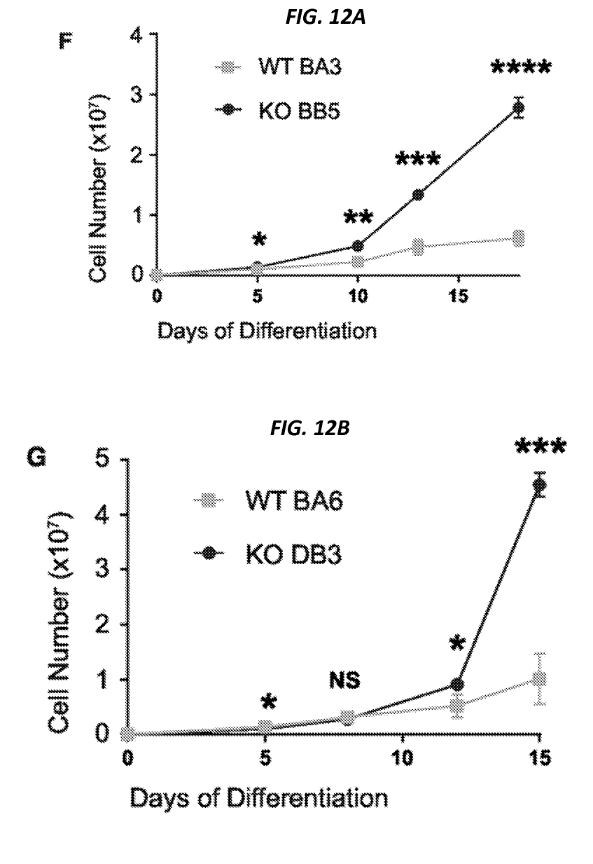

Herein, the inventors have also used CRISPR/Cas9-mediated genome editing to engineer isogenic hESC lines with either homozygous frame-shift deletions in SH2B3 or intact WT alleles (FIGS. 16E and 16F) (Ding et al., 2013a, 2013b; Gupta and Musunuru, 2014; Veres et al., 2014). The hESCs were differentiated in vitro to generate multipotent hematopoietic progenitor cells (HPCs) with erythroid, megakaryocytic, and myeloid potential (Kennedyet al., 2012; Mills et al., 2014; Sturgeon et al., 2013) (FIG. 14E). No differences in the yield of multipotent CD41a+/CD235a+ HPCs were noted between differentiating WT and SH2B3 knockout hESCs (Mills et al., 2014). However, in multiple independent isogenic hESC lines studied, loss of SH2B3 augmented erythroid cell production more than 3-fold (FIG. 12A, FIG. 12B and FIG. 16G).

Importantly, maturing WT and SH2B3 knockout erythroblasts exhibited similar morphologies, expression of erythroid cell surface markers, and globin gene expression patterns (FIG. 13D, FIG. 15E and FIG. 16H). While PSC erythroid differentiation protocols continue to undergo refinement, are not yet optimized for RBC manufacture, and primarily produce the earliest waves of hematopoiesis equivalent to those that developmentally arise from the yolk sac (Kobari et al., 2012; Slukvin, 2013; Sturgeon et al., 2014), the current findings demonstrate that stable perturbation of SH2B3 can allow for improved erythroid differentiation and expansion.

It is estimated that a unit of RBCs produced from HSPCs would cost $8,000-$15,000 using current in vitro differentiation protocols, while normal donor-derived units cost less than one-tenth of this amount (Migliaccio et al., 2012; Rousseau et al., 2014). While improvements in culture methods and use of systems such as bioreactors may be extremely valuable to improve upon this process, intrinsic alteration of stem cell sources to stimulate erythropoiesis should optimize the process further. To the best of our knowledge this has not been previously tested.

Here, the inventors demonstrate that by perturbing the SH2B3 gene, e.g., inhibiting SH2B3 function can be used to improve the ability of stem-cell-derived hematopoietic progenitors to expand and differentiate into the erythroid lineage. Importantly, the inventors demonstrate herein that the improvement of both expansion and differentiation by inhibiting SH3B3 surpasses the maximal expansion that could normally be achieved by simply optimizing cytokine concentrations in such cultures, which illustrates the complementary benefit of targeting intrinsic regulatory pathways. The increase in yields resulting from SH2B3 suppression allows RBCs to be produced from HSPCs at an estimated cost that is less than one-fifth of current approaches and with fewer starting stem cells. Thus, a single RBC unit is estimated to require 25 million CD34 HSPCs and perturbation of SH2B3 should reduce the required number of starting cells to 5 million (Migliaccio et al., 2012). With additional technological advances it is likely that both the cost and the efficiency of this process can be improved further. Importantly, the inventors discovery suggest that inhibition of SH2B3 improves erythroid differentiation from a variety of human stem cell sources, regardless of the differentiation method employed, and that this process can be used for therapeutic purposes and assist the ever-increasing demand on the blood supply (Williamson and Devine, 2013).

### REFERENCES

All references cited in the specification and Examples are incorporated herein in their entirety by reference.
Abecasis, G. R., Altshuler, D., Auton, A., Brooks, L. D., Durbin, R. M., Gibbs, R. A., Hurles, M. E., and McVean, G. A. (2010). A map of human genome variation from population-scale sequencing. Nature 467, 1061-1073.
Abkowitz, J. L., Sabo, K. M., Nakamoto, B., Blau, C. A., Martin, F. H., Zsebo, K. M., and Papayannopoulou, T. (1991). Diamond-blackfan anemia: in vitro response of erythroid progenitors to the ligand for c-kit. Blood 78, 2198-2202.
Adzhubei, I. A., Schmidt, S., Peshkin, L., Ramensky, V. E., Gerasimova, A., Bork, P., Kondrashov, A. S., and Sunyaev, S. R. (2010). A method and server for predicting damaging missense mutations. Nat Methods 7, 248-249.
Bersenev, A., Wu, C., Balcerek, J., and Tong, W. (2008). Lnk controls mouse hematopoietic stem cell self-renewal and quiescence through direct interac [symbol] tions with JAK2. J. Clin. Invest. 118, 2832-2844.
Carvalho, B. S., and Irizarry, R. A. (2010). A framework for oligonucleotide microarray preprocessing. Bioinformatics 26, 2363-2367.
Chun, S., and Fay, J. C. (2009). Identification of deleterious mutations within three human genomes. Genome Res 19, 1553-1561.
Cingolani, P., Platts, A., Wang le, L., Coon, M., Nguyen, T., Wang, L., Land, S. J., Lu, X., and Ruden, D. M. (2012). A program for annotating and predicting the effects of single nucleotide polymorphisms, SnpEff: SNPs in the genome of Drosophila melanogaster strain w1118; iso-2; iso-3. Fly (Austin) 6, 80-92.
d'Onofrio, G., Chirillo, R., Zini, G., Caenaro, G., Tommasi, M., and Micciulli, G. (1995). Simultaneous measurement of reticulocyte and red blood cell indices in healthy subjects and patients with microcytic and macrocytic anemia. Blood 85, 818-823.
Ding, Q., Lee, Y. K., Schaefer, E. A., Peters, D. T., Veres, A., Kim, K., Kuperwasser, N., Motola, D. L., Meissner, T. B., Hendriks, W. T., et al. (2013a). A TALEN genome-editing system for generating human stem cell-based dis [symbol] ease models. Cell Stem Cell 12, 238-251.
Ding, Q., Regan, S. N., Xia, Y., Oostrom, L. A., Cowan, C. A., and Musunuru, K. (2013b) Enhanced efficiency of human pluripotent stem cell genome editing through replacing TALENs with CRISPRs. Cell Stem Cell 12, 393-394.
Fox, I. J., Daley, G. Q., Goldman, S. A., Huard, J., Kamp, T. J., and Trucco, M. (2014). Stem cell therapy. Use of differentiated pluripotent stem cells as replacement therapy for treating disease. Science 345, 1247391.
Gery, S., and Koeffler, H. P. (2013). Role of the adaptor protein LNK in normal and malignant hematopoiesis. Oncogene 32, 3111-3118.
Giarratana, M. C., Rouard, H., Dumont, A., Kiger, L., Safeukui, I., Le Pennec, P. Y., Franc,ois, S., Trugnan, G., Peyrard, T., Marie, T., et al. (2011). Proof of principle for transfusion of in vitro-generated red blood cells. Blood 118, 5071-5079.
Gupta, R. M., and Musunuru, K. (2014). Expanding the genetic editing tool kit: ZFNs, TALENs, and CRISPR-Cas9. J. Clin. Invest. 124, 4154-4161.
Hirose, S., Takayama, N., Nakamura, S., Nagasawa, K., Ochi, K., Hirata, S., Yamazaki, S., Yamaguchi, T., Otsu, M., Sano, S., et al. (2013). Immortalization of erythroblasts by c-MYC and BCL-XL enables large-scale erythrocyte production from human pluripotent stem cells. Stem Cell Reports 1, 499-508.
Huang, X., Shah, S., Wang, J., Ye, Z., Dowey, S. N., Tsang, K. M., Mendelsohn, L. G., Kato, G. J., Kickler, T. S., and Cheng, L. (2014). Extensive ex vivo expan [symbol] sion of functional human erythroid precursors established from umbilical cord blood cells by defined factors. Mol. Ther. 22, 451-463.
Huang, X., Wang, Y., Yan, W., Smith, C., Ye, Z., Wang, J., Gao, Y., Mendelsohn, L., and Cheng, L. (2015). Production of Gene-Corrected Adult Beta Globin Protein in Human Erythrocytes Differentiated from Patient iPSCs After Genome Editing of the Sickle Point Mutation. Stem Cells 33, 1470-1479.
Kennedy, M., Awong, G., Sturgeon, C. M., Ditadi, A., LaMotte-Mohs, R., Zu´n~iga-Pflu\ddot{}cker, J. C., and Keller, G. (2012). T lymphocyte potential marks the emergence of definitive hematopoietic progenitors in human pluripotent stem cell differentiation cultures. Cell Rep. 2, 1722-1735.
King, M. J., Behrens, J., Rogers, C., Flynn, C., Greenwood, D., and Chambers, K. (2000). Rapid flow cytometric test for the diagnosis of membrane cytoskeleton-associated haemolytic anaemia. Br J Haematol 111, 924-933.
Kobari, L., Yates, F., Oudrhiri, N., Francina, A., Kiger, L., Mazurier, C., Rouzbeh, S., El-Nemer, W., Hebert, N., Giarratana, M. C., et al. (2012). Human induced pluripotent stem cells can reach complete terminal matura [symbol] tion: in vivo and in vitro evidence in the erythropoietic differentiation model. Haematologica 97, 1795-1803.
Kumar, P., Henikoff, S., and Ng, P. C. (2009). Predicting the effects of coding non-synonymous variants on protein function using the SIFT algorithm. Nat Protoc 4, 1073-1081.
Kurita, R., Suda, N., Sudo, K., Miharada, K., Hiroyama, T., Miyoshi, H., Tani, K., and Nakamura, Y. (2013). Establishment of immortalized human erythroid progenitor cell lines able to produce enucleated red blood cells. PLoS ONE 8, e59890.
Lasho, T. L., Pardanani, A., and Tefferi, A. (2010). LNK mutations in JAK2 mu [symbol] tation-negative erythrocytosis. N. Engl. J. Med. 363, 1189-1190.
Lee, H. Y., Gao, X., Barrasa, M. I., Li, H., Elmes, R. R., Peters, L. L., and Lodish, H. F. (2015). PPAR-alpha and glucocorticoid receptor synergize to promote erythroid progenitor self-renewal. Nature.
Li, B., and Leal, S. M. (2008). Methods for detecting associations with rare variants for common diseases: application to analysis of sequence data. Am J Hum Genet 83, 311-321.
Ludwig, L. S., Gazda, H. T., Eng, J. C., Eichhorn, S. W., Thiru, P., Ghazvinian, R., George, T. I., Gotlib, J. R., Beggs, A. H., Sieff, C. A., et al. (2014). Altered trans [symbol] lation of GATA1 in Diamond-Blackfan anemia. Nat. Med. 20, 748-753.
McKenna, A., Hanna, M., Banks, E., Sivachenko, A., Cibulskis, K., Kernytsky, A., Garimella, K., Altshuler, D., Gabriel, S., Daly, M., et al. (2010). The Genome Analysis Toolkit: a MapReduce framework for analyzing next-generation DNA sequencing data. Genome Res 20, 1297-1303.
McMullin, M. F., Wu, C., Percy, M. J., and Tong, W. (2011). A nonsynonymous LNK polymorphism associated with idiopathic erythrocytosis. Am. J. Hematol. 86, 962-964.
Merryweather-Clarke, A. T., Atzberger, A., Soneji, S., Gray, N., Clark, K., Waugh, C., McGowan, S. J., Taylor, S., Nandi, A. K., Wood, W. G., et al. (2011). Global gene expression analysis of human erythroid progenitors. Blood 117, e96-108.
Migliaccio, A. R., Whitsett, C., Papayannopoulou, T., and Sadelain, M. (2012). The potential of stem cells as an in vitro source of red blood cells for transfu [symbol] sion. Cell Stem Cell 10, 115-119.
Mills, J. A., Paluru, P., Weiss, M. J., Gadue, P., and French, D. L. (2014). Hematopoietic differentiation of pluripotent stem cells in culture. Methods Mol. Biol. 1185, 181-194.
Moraga, I., Wernig, G., Wilmes, S., Gryshkova, V., Richter, C. P., Hong, W. J., Sinha, R., Guo, F., Fabionar, H., Wehrman, T. S., et al. (2015) Tuning cytokine receptor signaling by re-orienting dimer geometry with surrogate ligands. Cell 160, 1196-1208.
Perrotta, S., Gallagher, P. G., and Mohandas, N. (2008). Hereditary spherocy-tosis. Lancet 372, 1411-1426.
Purcell, S. M., Moran, J. L., Fromer, M., Ruderfer, D., Solovieff, N., Roussos, P., O'Dushlaine, C., Chambert, K., Bergen, S. E., Kahler, A., et al. (2014). A polygenic burden of rare disruptive mutations in schizophrenia. Nature 506, 185-190.
Ritchie, M. E., Phipson, B., Wu, D., Hu, Y., Law, C. W., Shi, W., and Smyth, G. K. (2015). limma powers
Rousseau, G. F., Giarratana, M. C., and Douay, L. (2014). Large-scale produc [symbol] tion of red blood cells from stem cells: what are the technical challenges ahead? Biotechnol. J. 9, 28-38.
Sankaran, V. G., and Orkin, S. H. (2013). The switch from fetal to adult hemoglo [symbol] bin. Cold Spring Harb. Perspect. Med. 3, a011643.
Sankaran, V. G., Ludwig, L. S., Sicinska, E., Xu, J., Bauer, D. E., Eng, J. C., Patterson, H. C., Metcalf, R. A., Natkunam, Y., Orkin, S. H., et al. (2012). Cyclin D3 coordinates the cell cycle during differentiation to regulate erythrocyte size and number. Genes Dev. 26, 2075-2087.
Sankaran, V. G., Menne, T. F., Scepanovic, D., Vergilio, J. A., Ji, P., Kim, J., Thiru, P., Orkin, S. H., Lander, E. S., and Lodish, H. F. (2011). MicroRNA-15a and -16-1 act via MYB to elevate fetal hemoglobin expression in human trisomy 13. Proc. Natl. Acad. Sci. USA 108, 1519-1524.
Sankaran, V. G., Menne, T. F., Xu, J., Akie, T. E., Lettre, G., Van Handel, B., Mikkola, H. K., Hirschhorn, J. N., Cantor, A. B., and Orkin, S. H. (2008). Human fetal hemoglobin expression is regulated by the developmental stage-specific repressor BCL11A. Science 322, 1839-1842.
Schwarz, J. M., Cooper, D. N., Schuelke, M., and Seelow, D. (2014). MutationTaster2: mutation prediction for the deep-sequencing age. Nat Methods 11, 361-362.
Slukvin, I. I. (2013). Hematopoietic specification from human pluripotent stem cells: current advances and challenges toward de novo generation of hemato-poietic stem cells. Blood 122, 4035-4046.
Spolverini, A., Pieri, L., Guglielmelli, P., Pancrazzi, A., Fanelli, T., Paoli, C., Bosi, A., Nichele, I., Ruggeri, M., and Vannucchi, A. M. (2013). Infrequent occurrence of mutations in the PH domain of LNK in patients with JAK2 mutation-negative ‘idiopathic’ erythrocytosis. Haematologica 98, e101-e102.
Sturgeon, C. M., Ditadi, A., Awong, G., Kennedy, M., and Keller, G. (2014). Wnt signaling controls the specification of definitive and primitive hematopoiesis from human pluripotent stem cells. Nat. Biotechnol. 32, 554-561.
Sturgeon, C. M., Ditadi, A., Clarke, R. L., and Keller, G. (2013). Defining the path to hematopoietic stem cells. Nat. Biotechnol. 31, 416-418.
Subramanian, A., Tamayo, P., Mootha, V. K., Mukherjee, S., Ebert, B. L., Gillette, M. A., Paulovich, A., Pomeroy, S. L., Golub, T. R., Lander, E. S., et al. (2005). Gene set enrichment analysis: a knowledge-based approach for interpreting genome-wide expression profiles. Proc Natl Acad Sci U S A 102, 15545-15550.
Tennessen, J. A., Bigham, A. W., O'Connor, T. D., Fu, W., Kenny, E. E., Gravel, S., McGee, S., Do, R., Liu, X., Jun, G., et al. (2012). Evolution and functional impact of rare coding variation from deep sequencing of human exomes. Science 337, 64-69.
van der Harst, P., Zhang, W., Mateo Leach, I., Rendon, A., Verweij, N., Sehmi, J., Paul, D. S., Elling, U., Allayee, H., Li, X., et al. (2012). Seventy-five genetic loci influencing the human red blood cell. Nature 492, 369-375.
Velazquez, L., Cheng, A. M., Fleming, H. E., Furlonger, C., Vesely, S., Bernstein, A., Paige, C. J., and Pawson, T. (2002). Cytokine signaling and hematopoietic homeostasis are disrupted in Lnk-deficient mice. J. Exp. Med. 195, 1599-1611.
Veres, A., Gosis, B. S., Ding, Q., Collins, R., Ragavendran, A., Brand, H., Erdin, S., Cowan, C. A., Talkowski, M. E., and Musunuru, K. (2014). Low incidence of off-target mutations in individual CRISPR-Cas9 and TALEN targeted human stem cell clones detected by whole-genome sequencing. Cell Stem Cell 15,27-30.
Williamson, L. M., and Devine, D. V. (2013). Challenges in the management of the blood supply. Lancet 381, 1866-1875.

1. A method of producing red blood cells (RBCs) ex vivo from a population of stem cells or progenitor cells, or both, the method comprising:
(i) inhibiting SH2B3 in the population of stem cells or progenitor cells, or both;
(ii) culturing the population of stem cells or progenitor cells, or both, for a time sufficient to induce differentiation of at least one stem cell or progenitor cell to a RBC; and
(iii) collecting a population of RBCs.

2. The method of claim 1, wherein the inhibiting decreases SH2B3 protein level, SH2B3 mRNA level, SH2B3 protein activity, or combinations thereof.

3. The method of claim 1, wherein the inhibiting comprises contacting the population of stem cells or progenitor cells, or both, with a genome-editing agent for targeted excision of the SH2B3 gene from at least one stem cell or progenitor cell.

4. The method of claim 3, wherein the genome-editing agent is selected from the group consisting of a Zinc-Finger Nuclease (ZFN), a Clustered Regularly Interspaced Short Palindromic Repeats (CRISPR)/CRISPR associated (Cas) system, and a Transcription Activator-Like Effector Nuclease (TALEN).

5. (canceled)

6. The method of claim 1, wherein the inhibiting comprises contacting the population of stem cells or progenitor cells, or both, with an antagonist of SH2B3, wherein the antagonist of SH2B3 is selected from the group consisting of an inorganic molecule, an organic molecule, a nucleic acid, a nucleic acid analog or derivative, a peptide, a peptidomimetic, a protein, an antibody or an antigen-binding fragment thereof, and combinations thereof.

7. (canceled)

8. The method of claim 6, wherein the antagonist of SH2B3 specifically binds to SH2 domain, PH domain, or both the SH2 and PH domains of the SH2B3 protein.

9. The method of claim 6, wherein the antagonist of SH2B3 is a nucleic acid RNAi agent that inhibits the expression of SH2B3.

10. (canceled)

11. The method of claim 1, wherein the population of stem cells or progenitor cells, or both, is selected from the group consisting of hematopoietic stem cells, hematopoietic progenitor cells, pluripotent stem cells, induced pluripotent stem cells (iPSCs), embryonic stem cells, and combinations thereof.

12. The method of claim 1, wherein the method increases the expansion of RBCs from the population of stem cells or progenitor cells, or both.

13. (canceled)

14. The method of claim 1, wherein the population of stem cells or progenitor cells is of mammalian origin or of human origin.

15. (canceled)

16. (canceled)

17. The method of claim 1, wherein the population of stem cells or progenitor cells, or both, are obtained from a donor subject.

18. The method of claim 17, wherein the population of stem cells or progenitor cells, or both, are derived from peripheral blood mononuclear cells, cord blood, bone marrow, cord tissue, or G-CSF mobilize peripheral blood of the donor subject.

19. The method of claim 18, further comprising administering a population of RBCs to a subject in need thereof, wherein the RBCs are produced from the population of stem cells and/or progenitor cells obtained from the donor subject.

20. The method of claim 19, wherein the population of stem cells is iPSCs.

21. A population of RBCs produced according to the method of claim 1.

22. The population of RBCs of claim 21, further comprising a population of stem cells or progenitor cells, or both.

23. (canceled)

24. The population of RBCs of claim 21, wherein the population of RBCs are prepared for cryogenic storage.

25. A cell culture media comprising a population of stem cells or progenitor cells, or both, at least one RBC differentiated from at least one stem cell or progenitor cell, and an antagonist of SH2B3.

26. (canceled)

27. (canceled )

28. A method of administering a population of RBCs to a subject, comprising administering an effective amount of RBCs to the subject, wherein the RBCs have been differentiated from a population of stem cells or progenitor cells, or both, which have been contacted ex vivo or in vitro with:
a. an effective amount of an antagonist of SH2B3, wherein the antagonist of SH2B3 decreases the activity of the SH2B3 protein or decreases SH2B3 mRNA or protein levels in the population of stem cells or progenitor cells, or both, to induce them to differentiate into a RBC, or
b. an effective amount of a genome-editing agent, wherein the genome-editing agent excises the SH2B3 gene from at least one stem cell or progenitor cell.

29. (canceled)
